(19) United States
(12) Patent Application Publication
(10) Pub. No.: US 2025/0163510 A1
(43) Pub. Date: May 22, 2025

(54) USE OF MICROVESICLE SIGNATURES IN THE IDENTIFICATION AND TREATMENT OF RENAL DISORDERS

(71) Applicants: Exosome Diagnostics, Inc., Waltham, MA (US); The Brigham and Women's Hospital, Inc., Boston, MA (US)

(72) Inventors: Johan Karl Olov SKOG, Waltham, MA (US); Brian C. HAYNES, Austin, TX (US); Elliot HALLMARK, Austin, TX (US); James HURLEY, Marblehead, MA (US); Jamil AZZI, Boston, MA (US)

(21) Appl. No.: 18/839,547

(22) PCT Filed: Feb. 21, 2023
(86) PCT No.: PCT/US2023/013486

§ 371 Date: Aug. 19, 2024

Related U.S. Application Data
(60) Provisional application No. 63/377,631, filed on Sep. 29, 2022.
(60) Provisional application No. 63/311,671, filed on Feb. 18, 2022.

(51) Int. Cl. C12Q 1/6883; G01N 33/68

(52) U.S. Cl. CPC C12Q 1/6883; G01N 33/6893; C12Q 2600/158; G01N 2800/245; G01N 2800/52

(57) ABSTRACT

The present disclosure relates to methods of identifying and treating kidney rejection in a subject comprising analyzing RNA, including microvesicular RNA.

### RELATED APPLICATIONS

This application claims priority to, and the benefit of, U.S. Provisional Application No. 63/311,671, filed Feb. 18, 2022, and U.S. Provisional Application No. 63/377,631, filed Sep. 29, 2022. The contents of each of the aforementioned patent applications are incorporated herein by reference in their entireties.

### GOVERNMENT SUPPORT

This invention was made with government support under Grant No. RO1-AI134842 awarded by the National Institutes of Health and under Grant No. F32DK11106 awarded by the National Institutes of Health. The government has certain rights in the invention.

### BACKGROUND

In 2021 in the United States alone, there were an estimated 25,000 kidney transplants. Although the introduction of more potent immunosuppressive drugs has decreased the incidence of acute rejection following transplantation, roughly 10% of kidney transplant patients will experience acute rejection within the first year, with 20-30% of transplants failing within 5 years and close to 50% failing within 10 years. Moreover, episodes of acute rejection, especially those that occur within the first year, are associated with poor long-term allograft outcome. The gold standard in the diagnosis of acute rejection following kidney rejection is kidney allograft biopsy followed by histopathological evaluation. However, such biopsies suffer from several limitations, including invasiveness, cost and inter-observer variability. Indeed, in addition to the increased costs, repeated biopsies for the monitoring of rejection (“protocol” biopsies) are associated with increased negative complications. Moreover, 70-95% of protocol biopsies are negative for rejection. Resultingly, most sites do not perform protocol biopsies and thus many early-stage rejections are missed. Attempts at developing an alternative to biopsies for the diagnosis of kidney transplant rejection have thus far failed. Serum creatinine (SCr) and urinary protein excretion are traditional biomarkers currently used to monitor the kidney graft function, but they lack sensitivity, specificity and predictive ability. There is an urgent need for an accurate, non-invasive method of identifying kidney transplant rejection, particularly at an early stage following transplant to both identify early rejections and rule out unnecessary biopsies.

Additionally, kidney transplant rejection can be broadly categorized as TCMR (T cell-mediated rejection, also referred to as cell-mediated kidney transplant rejection) or ABMR (antibody-mediated rejection, also referred to as antibody-mediated kidney transplant rejection). Instances in which both TCMR and ABMR were present within the same kidney transplant patient have also been reported. As the treatment plans for TCMR and ABMR can differ, identification of the particular rejection subtype in a subject is a critical aspect of kidney transplant rejection treatment. Historically, biopsies have been required to distinguish between the presence of TCMR, ABMR or both TCMR and ABMR. Accordingly, there is also an urgent need of accurate, non-invasive methods of identifying the subtype of kidney transplant rejection in subject that has been identified as having a kidney transplant rejection or is suspected of having a kidney transplant rejection.

Finally, inflammation of the kidneys has been identified as a pathogenic mechanism for a variety of different kidney diseases and disorders, including in subjects who have previously undergone a kidney transplant rejection. Such diseases and disorders include, but are not limited to, urinary tract infections, BK viremia and CMV viremia. Thus, there is also an urgent need of accurate, non-invasive methods of identifying kidney inflammation and identifying subjects who are at risk of developing kidney inflammation.

### SUMMARY

The present disclosure provides a method of determining the risk of a kidney transplant rejection in a subject who has undergone a kidney transplant, the method comprising: a) determining the expression level of at least two of three biomarkers and at least one endogenous control gene in RNA isolated from a biological sample from the subject, wherein the three biomarkers comprise IL32, B2M and CXCL11; b) normalizing the expression of the biomarkers to the expression level of the at least one endogenous control gene; c) inputting the normalized expression levels from step (b) into an algorithm to generate a score; and d) determining the risk of a kidney transplant rejection in the subject based on the score.

The present disclosure provides a method of determining the risk of a kidney inflammation in a subject, the method comprising: a) determining the expression level of at least two of three biomarkers and at least one endogenous control gene in RNA isolated from a biological sample from the subject, wherein the three biomarkers comprise IL32, B2M and CXCL11; b) normalizing the expression of the biomarkers to the expression level of the at least one endogenous control gene; c) inputting the normalized expression levels from step (b) into an algorithm to generate a score; and d) determining the risk of kidney inflammation in the subject in the subject based on the score. In some aspects, the subject has undergone a kidney transplant.

In some aspects, the subject is a subject who has been identified to be at risk for a kidney transplant rejection based on at least one clinical indications of kidney transplant rejection, preferably wherein the at least one clinical indication of kidney transplant rejection comprises increased serum creatinine.

In some aspects of the preceding methods, step (a) comprises determining the expression level of each of the three biomarkers.

In some aspects of the preceding methods, determining the risk of a kidney transplant rejection in the subject based on the score comprises: i) comparing the score to a predetermined cutoff value; and ii) determining that the at the subject is at a high risk of having a kidney transplant rejection when the score is greater than or equal to the predetermined cutoff value or determining that the subject is at low risk of having a kidney transplant rejection when the score is less than the predetermined cutoff value.

In some aspects, the algorithm is the product of a feature selection wrapper algorithm, a machine learning algorithm, a trained classifier built from at least one predictive classification algorithm or any combination thereof, preferably wherein the algorithm is the product of a trained classifier built from at least one predictive classification algorithm, wherein the at least one predictive classification algorithm comprises SVM-linear.

In some aspects, the trained classifier is trained using the expression levels of the biomarkers measured in RNA isolated from a training set of biological samples, wherein the training set of biological sample comprises: i) a first plurality of biological samples isolated from subjects identified to be at risk for a kidney transplant rejection based on at least one clinical indications of kidney transplant rejection, preferably wherein the at least one clinical indication of kidney transplant rejection comprises increased serum creatinine.

In some aspects, a portion of the biological samples in the first plurality of biological samples are from subjects who are identified by biopsy to be positive for kidney transplant rejection, and a portion of the biological samples in the first plurality of biological samples are from subjects who are identified by biopsy to be negative for kidney transplant rejection.

In some aspects, the training set of biological samples further comprises: ii) a second plurality of biological samples isolated from subjects that have no clinical indications of a kidney transplant rejection, preferably wherein the biological samples in the second plurality are from subjects identified by biopsy to be negative for kidney transplant rejection.

In some aspects of the preceding methods, the algorithm and the predetermined cutoff value has i) a sensitivity for identifying kidney transplant rejection of at least about 90%; and ii) a negative predictive value for identifying kidney transplant rejection of at least about 93%.

The present disclosure provides a method of determining the risk of a kidney transplant rejection in a subject who has undergone a kidney transplant, the method comprising: a) determining the expression level of at least two of six biomarkers and at least one endogenous control gene in RNA isolated from a biological sample from the subject, wherein the six biomarkers comprise IFNAR2, PYCARD, CD44, IRAK2, B2M and NAMPT; b) normalizing the expression of the biomarkers to the expression level of the at least one endogenous control gene; c) inputting the normalized expression levels from step (b) into an algorithm to generate a score; and d) determining the risk of a kidney transplant rejection in the subject based on the score. In some aspects, the subject is a subject that has no clinical indications of a kidney transplant rejection.

In some aspects of the preceding methods, step (a) comprises determining the expression level of: a) at least three of the six biomarkers; b) at least four of the six biomarkers; c) at least five of the six biomarkers; or d) each of the six biomarkers.

In some aspects, determining the risk of a kidney transplant rejection in the subject based on the score comprises: i) comparing the score to a predetermined cutoff value; and ii) determining that the at the subject is at a high risk of having a kidney transplant rejection when the score is greater than or equal to the predetermined cutoff value or determining that the subject is at low risk of having a kidney transplant rejection when the score is less than the predetermined cutoff value.

In some aspects, the algorithm is the product of a feature selection wrapper algorithm, a machine learning algorithm, a trained classifier built from at least one predictive classification algorithm or any combination thereof, preferably wherein the algorithm is the product of: a first trained classifier built from at least one predictive classification algorithm, wherein the at least one predictive classification algorithm comprises naïve Bayes; and an at least second trained classifier built from at least one predictive classification algorithm, wherein the at least one predictive classification algorithm comprises naïve Bayes.

In some aspects, the first and/or the at least second trained classifier(s) is/are trained using the expression levels of the biomarkers measured in RNA isolated from a training set of biological samples, wherein the training set of biological sample comprises: i) a first plurality of biological samples isolated from subjects that have no clinical indications of a kidney transplant rejection. In some aspects, a portion of the biological samples in the first plurality of biological samples are from subjects who are identified by biopsy to be positive for kidney transplant rejection, and a portion of the biological samples in the first plurality of biological samples are from subjects who are identified by biopsy to be negative for kidney transplant rejection.

In some aspects, the training set of biological samples further comprises: ii) a second plurality of biological samples isolated from subjects identified to be at risk for a kidney transplant rejection based on at least one clinical indications of kidney transplant rejection, preferably wherein the biological samples in the second plurality are from subjects identified by biopsy to be positive for kidney transplant rejection.

In some aspects, the first trained classifier has a sensitivity for identifying kidney transplant rejection of at least about 90%. In some aspects, the at least second trained classifier has a specificity for identifying kidney transplant rejection of at least about 90%.

In some aspects, the algorithm and the predetermined cutoff value has: i) a sensitivity for identifying kidney transplant rejection of at least about 93%; ii) a specificity for identifying kidney transplant rejection of at least about 48%; and iii) a negative predictive value for identifying kidney transplant rejection of at least about 97%.

In some aspects, the algorithm and the predetermined cutoff value has: i) a sensitivity for identifying kidney transplant rejection of at least about 61%; ii) a specificity for identifying kidney transplant rejection of at least about 84%; and iii) a negative predictive value for identifying kidney transplant rejection of at least about 90%.

The present disclosure provides a method of determining the risk of antibody-mediated rejection (ABMR) in a subject that has undergone a kidney transplant, the method comprising: a) determining the expression level of at least two of five biomarkers and at least one endogenous control gene in RNA isolated from a biological sample from the subject, wherein the five biomarkers comprise IL18BP, CXCL11, CD74, CD44 and C3; b) normalizing the expression of the biomarkers to the expression level of the at least one endogenous control gene; c) inputting the expression levels from step (b) into an algorithm to generate a score; and d) identifying the risk of ABMR in the subject based on the score. In some aspects, the subject is a subject who has been identified as having a kidney transplant rejection or who has been identified as being at high risk for kidney transplant rejection.

In some aspects, step (d) comprises: i) comparing the score to a predetermined cutoff value; and ii) identifying the risk of ABMR in the subject based on relationship between the score and the predetermined cutoff value.

In some aspects of the preceding methods, step (a) comprises determining the expression level: a) at least three of the five biomarkers; or b) at least four of the five biomarkers. In some aspects, step (a) comprises determining the expression level of each of the five biomarkers.

In some aspects of the preceding methods, the algorithm is a product of a feature selection wrapper algorithm, machine learning algorithm, trained classifier, logistic regression model or any combination thereof, that was trained using: a) the expression levels of the at least one, or the at least two, or the at least three, or the at least four or each of the biomarkers in at least one biological sample from at least one subject who has ABMR; and b) the expression levels of the at least one, or the at least two, or the at least three, or the at least four or each of the biomarkers in at least one biological sample from at least one subject who has TCMR.

In some aspects, the algorithm and the predetermined cutoff value has: i) a sensitivity for ruling out ABMR of at least about 77%; ii) a specificity for ruling out ABMR of at least about 62%; iii) a negative predictive value for ruling out ABMR of at least about 90%; and iv) a positive predictive value for ruling out ABMR of at least about 38%.

In some aspects of the preceding methods, the predictive classification algorithm, the feature selection wrapper algorithm, and/or the machine learning algorithm comprises XGBoost (XGB), random forest (RF), Lasso and Elastic-Net Regularized Generalized Linear Models (glmnet), Linear Discriminant Analysis (LDA), cforest, classification and regression tree (CART), treebag, k nearest-neighbor (knn), neural network (nnet), support vector machine-radial (SVM-radial), support vector machine-linear (SVM-linear), naïve Bayes (NB), multilayer perceptron (mlp), Boruta or any combination thereof.

In some aspects of the preceding methods, the RNA isolated from a biological sample from the subject and/or the RNA isolated from a training set of biological samples comprises cell-free RNA, microvesicular RNA or any combination thereof.

In some aspects of the preceding methods, the biological sample from the subject and/or the biological samples in the training sets is/are urine samples.

In some aspects of the preceding methods, the at least one endogenous control gene comprises PGK1.

In some aspects of the preceding methods, determining the expression level of a biomarker comprises quantitative PCR (qPCR), quantitative real-time PCR, semi-quantitative real-time PCR, reverse transcription PCR (RT-PCR), reverse transcription quantitative PCR (qRT-PCR), microarray analysis, sequencing, next-generation sequencing (NGS), high-throughput sequencing, direct-analysis, droplet digital PCR, or any combination thereof.

In some aspects, the preceding methods can further comprise: i) performing a kidney biopsy on the subject; ii) administering at least one kidney transplant rejection therapy to the subject; iii) administering at least one TCMR-targeted therapy to the subject; iv) administering at least one ABMR-targeted therapy to the subject; and/or v) administering at least one kidney inflammation therapy to the subject.

Any of the aspects and embodiments described above and herein can be combined with any other aspect and embodiment described above and herein.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

The above and further features will be more clearly appreciated from the following detailed description when taken in conjunction with the accompanying drawings.

FIG. 1 is a diagram showing the composition of the study sample cohort categorized by biopsy type, rejection status and rejection subtype diagnosis.

FIG. 2A shows the receiver-operating characteristic curve analysis and corresponding area under the curve (AUC) for the 3-gene signature using the linear support vector machine (SVM) classifier of the present disclosure on the cohort of 182 for-cause biopsy associated samples.

FIG. 2B shows the receiver-operating characteristic curve analysis and corresponding area under the curve (AUC) by the standard method of ΔeGFR on the cohort of 182 for-cause biopsy associated samples.

FIG. 3 is a waterfall plot of the 3-gene signature using the linear SVM classifier scores for identifying any-cause kidney transplant rejection on the cohort of 182 for-cause biopsy associated samples as described in Example 1 of the present disclosure. The classifier threshold is annotated as the horizontal green line.

FIG. 4 is a box and whiskers plot depicting the ability of the linear SVM classifier scores (y-axis) to distinguish between non-rejection and different types of subclinical rejection on the cohort of 182 for-cause biopsy associated samples.

FIG. 5A shows the receiver-operating characteristic curve analysis and corresponding area under the curve (AUC) for the 6-gene signature using the ensemble classifier of the present disclosure on the cohort of 216 management biopsy associated samples.

FIG. 5B shows the receiver-operating characteristic curve analysis and corresponding area under the curve (AUC) by the standard method of ΔeGFR on the cohort of 216 management biopsy associated samples.

FIG. 6 is a waterfall plot of the 6-gene signature using the ensemble classifier scores for identifying any-cause kidney transplant rejection as described in Example 1 of the present disclosure on the cohort of 216 management biopsy associated samples.

FIG. 7A is a waterfall plot of the 3-gene signature using the ensemble classifier scores for identifying any-cause rejection or significant inflammation as described in Example 3 of the present disclosure on the cohort of 182 clinically indicated biopsy associated samples.

FIG. 7B is an analysis of the rates of adverse outcomes in patients with false positive test (high classifier score but biopsy showing no rejection or inflammation) compared to patients with true negative signature (low classifier score and biopsy showing no rejection or inflammation) for which follow-up data up to three years post biopsy was available.

FIG. 8 is a waterfall plot of the 3-gene signature using the ensemble classifier scores in samples with any-cause rejection for varying degrees of inflammation. Six samples that were rejection negative at the time of urine collection that progressed to rejection within six months are annotated with an asterisk.

FIG. 9 shows the receiver-operating characteristic curve analysis and corresponding area under the curve (AUC) for the 5-gene signature described in Example 2 herein.

FIG. 10 is a waterfall plot of the 5-gene signature described in Example 2 of the present disclosure. The classifier threshold is annotated as the horizontal line and the ABMR samples are denoted with an arrow. All other samples are TCMR samples.

### DETAILED DESCRIPTION

Chronic kidney disease (CKD) is a major health concern in the United States and worldwide. While patients with end-stage kidney disease (ESKD) require either dialysis or transplantation to sustain their lives, the latter remains the treatment of choice. However, long term graft survival remains a major challenge due mostly to acute and chronic rejection. Although the rate of acute rejection has decreased in the modern era of potent immunosuppression, recent reported incidence of acute rejections in the literature ranges from 11 to 26%. During the first year after transplantation, the incidence of acute rejection is around 7.9%. This has been associated with a poor long-term allograft survival. The implementation of the Banff classification in 1991 provided a valuable tool for histopathological diagnosis of kidney transplant injury and allowed for standardization when comparing biopsy results between different studies. Serum creatinine (SCr), estimated glomerular filtration rate (eGFR) and urinary protein excretion are traditional biomarkers currently used to monitor the kidney allograft but they lack sensitivity, specificity and predictive ability. Kidney allograft biopsies with histopathological evaluation remain the gold standard to diagnose acute rejection. However, there are limitations to their use as they are invasive, costly and can be associated with significant morbidity. Several biomarkers have been identified as potential non-invasive tools to early diagnose graft rejection such as cell mRNA isolated from urine pellet. Recently, donor-derived cell-free DNA (dd-cfDNA) has been introduced to the clinical practice as a novel biomarker for graft rejection after solid organ transplantation. Despite results showing good performances in discriminating active rejection from no-rejection status, biopsies with T-cell mediated rejection (TCMR) subclass IA did not reach the 1% dd-cfDNA cut-off required for diagnosis.

The present disclosure provides methods of identifying and treating kidney rejection (including early kidney rejection) in a subject comprising analyzing microvesicular RNA, cell-free DNA or the combination of microvesicular and cell-free DNA. These methods allow for the selection of a treatment and/or treatment of an individual identified as having a kidney transplant rejection without the need for a renal biopsy, which can be an expensive, painful, and potentially dangerous procedure. Advantageously, the methods of the present disclosure can allow for the ruling out of unnecessary biopsies in lieu of burdensome and potentially harmful protocol biopsies. Moreover, non-invasive urine sample enables at-home collection, which is more convenient to the patient.

Extracellular membrane vesicles called microvesicles are shed by eukaryotic and prokaryotic cells, or budded off from the plasma membrane, to the exterior of the cell. These extracellular membrane vesicles are heterogeneous in size with diameters ranging from about 10 nm to about 5000 nm. As used herein, the term “microvesicle” encompasses all extracellular membrane vesicles with diameters ranging from about 10 nm to about 5000 nm, including those with diameters <0.8 μm. These extracellular membrane vesicles can include, but are not limited to, microvesicles, microvesicle-like particles, prostasomes, dexosomes, texosomes, ectosomes, oncosomes, apoptotic bodies, retrovirus-like particles, and human endogenous retrovirus (HERV) particles. As used herein, the term “microvesicle” also encompasses small microvesicles (approximately 10 to 1000 nm, and more often 30 to 200 nm in diameter) that are released by exocytosis of intracellular multivesicular bodies. Such small microvesicles are also sometimes referred to in the art as exosomes. As such, the terms “exosomes”, “extracellular vesicles”, “extracellular membrane vesicles”, and “microvesicles” are used interchangeably herein.

Microvesicles are known to contain nucleic acids, including various DNA and RNA types such as mRNA (messenger RNA), miRNA (micro RNA), tRNA (transfer RNA), piRNA (piwi-interacting RNA), snRNA (small nuclear RNA), snoRNA (small nucleolar RNA), and rRNA (ribosomal RNA), various classes of long non-coding RNA, including long intergenic non-coding RNA (lincRNA) as well as proteins. Recent studies reveal that nucleic acids within microvesicles have a role as biomarkers. For example, WO 2009/100029 describes, among other things, the use of nucleic acids extracted from microvesicles in Glioblastoma multiforme (GBM, a particularly aggressive form of cancer) patient serum for medical diagnosis, prognosis and therapy evaluation. WO 2009/100029 also describes the use of nucleic acids extracted from microvesicles in human urine for the same purposes. The use of nucleic acids extracted from microvesicles is considered to potentially circumvent the need for biopsies, highlighting the enormous diagnostic potential of microvesicle biology (Skog et al. Nature Cell Biology, 2008, 10(12): 1470-1476).

Microvesicles can be isolated from liquid biopsy samples from a subject, involving biofluids such as whole blood, serum, plasma, urine, and cerebrospinal fluid (CSF). The nucleic acids contained within the microvesicles can subsequently be extracted. The extracted nucleic acids, e.g., microvesicular RNA (also referred to as exosomal RNA), can be further analyzed based on detection of a biomarker or a combination of biomarkers. The analysis can be used to generate a clinical assessment that diagnoses a subject with a disease, predicts the disease outcome of the subject, stratifies the subject within a larger population of subjects, predicts whether the subject will respond to a particular therapy, or determines if a subject is responding to an administered therapy.

### Classifier #1

The present disclosure provides a method of determining the risk of a kidney transplant rejection in a subject who has undergone a kidney transplant, the method comprising: a) determining the expression level of at least two of three biomarkers and at least one endogenous control gene in RNA isolated from a biological sample from the subject, wherein the three biomarkers comprise IL32, B2M, and CXCL11; b) normalizing the expression of the biomarkers to the expression level of the at least one endogenous control gene; c) inputting the normalized expression levels from step (b) into an algorithm to generate a score; and d) determining the risk of a kidney transplant rejection in the subject based on the score.

The present disclosure provides a method of determining the risk of a kidney transplant rejection in a subject who has undergone a kidney transplant, the method comprising: a) determining the expression level of at least two of three biomarkers and at least one endogenous control gene in RNA isolated from a biological sample from the subject, wherein the three biomarkers comprise IL32, B2M, and CXCL11; b) normalizing the expression of the biomarkers to the expression level of the at least one endogenous control gene; and c) determining the risk of a kidney transplant rejection in the subject based on the normalized expression levels from step (b).

The present disclosure provides a method of determining the risk of a kidney transplant rejection in a subject who has undergone a kidney transplant, the method comprising: a) determining the expression level of at least two of three biomarkers in RNA isolated from a biological sample from the subject, wherein the three biomarkers comprise IL32, B2M, and CXCL11; b) inputting the expression levels from step (a) into an algorithm to generate a score; and c) determining the risk of a kidney transplant rejection in the subject based on the score.

The present disclosure provides a method of determining the risk of a kidney transplant rejection in a subject who has undergone a kidney transplant, the method comprising: a) determining the expression level of at least two of three biomarkers in RNA isolated from a biological sample from the subject, wherein the three biomarkers comprise IL32, B2M, and CXCL11; and b) determining the risk of a kidney transplant rejection in the subject based on the expression levels from step (a).

In some aspects, the present disclosure provides a method of determining that a subject who has undergone kidney transplant is undergoing a transplant rejection, the method comprising: a) determining the expression level of at least two of three biomarkers and at least one endogenous control gene in RNA isolated from a biological sample from the subject, wherein the three biomarkers comprise IL32, B2M, and CXCL11; b) normalizing the expression of the biomarkers to the expression level of the at least one endogenous control gene; c) inputting the normalized expression levels from step (b) into an algorithm to generate a score; and d) determining that the subject is undergoing kidney transplant rejection based on the score.

The present disclosure provides a method of determining that a subject who has undergone kidney transplant is undergoing a transplant rejection, the method comprising: a) determining the expression level of at least two of three biomarkers and at least one endogenous control gene in RNA isolated from a biological sample from the subject, wherein the three biomarkers comprise IL32, B2M, and CXCL11; b) normalizing the expression of the biomarkers to the expression level of the at least one endogenous control gene; and c) determining that the subject is undergoing kidney transplant rejection based on the normalized expression levels from step (b).

The present disclosure provides a method of determining that a subject who has undergone kidney transplant is undergoing a transplant rejection, the method comprising: a) determining the expression level of at least two of three biomarkers in RNA isolated from a biological sample from the subject, wherein the three biomarkers comprise IL32, B2M, and CXCL11; b) inputting the expression levels from step (a) into an algorithm to generate a score; and c) determining that the subject is undergoing kidney transplant rejection based on the score.

The present disclosure provides a method of determining that a subject who has undergone kidney transplant is undergoing a transplant rejection, the method comprising: a) determining the expression level of at least two of three biomarkers in RNA isolated from a biological sample from the subject, wherein the three biomarkers comprise IL32, B2M, and CXCL11; and b) determining that the subject is undergoing kidney transplant rejection based on the expression levels from step (a).

In some aspects, the RNA isolated from a biological sample from the subject can comprise cell-free RNA. In some aspects, the RNA isolated from a biological sample from the subject can comprise microvesicular RNA.

Accordingly, the present disclosure provides a method of determining the risk of a kidney transplant rejection in a subject who has undergone a kidney transplant, the method comprising: a) determining the expression level of at least two of three biomarkers and at least one endogenous control gene in microvesicular RNA isolated from a biological sample from the subject, wherein the three biomarkers comprise IL32, B2M, and CXCL11; b) normalizing the expression of the biomarkers to the expression level of the at least one endogenous control gene; c) inputting the normalized expression levels from step (b) into an algorithm to generate a score; and d) determining the risk of a kidney transplant rejection in the subject based on the score.

The present disclosure provides a method of determining the risk of a kidney transplant rejection in a subject who has undergone a kidney transplant, the method comprising: a) determining the expression level of at least two of three biomarkers and at least one endogenous control gene in microvesicular RNA isolated from a biological sample from the subject, wherein the three biomarkers comprise IL32, B2M, and CXCL11; b) normalizing the expression of the biomarkers to the expression level of the at least one endogenous control gene; and c) determining the risk of a kidney transplant rejection in the subject based on the normalized expression levels from step (b).

The present disclosure provides a method of determining the risk of a kidney transplant rejection in a subject who has undergone a kidney transplant, the method comprising: a) determining the expression level of at least two of three biomarkers in microvesicular RNA isolated from a biological sample from the subject, wherein the three biomarkers comprise IL32, B2M, and CXCL11; b) inputting the expression levels from step (a) into an algorithm to generate a score; and c) determining the risk of a kidney transplant rejection in the subject based on the score.

The present disclosure provides a method of determining the risk of a kidney transplant rejection in a subject who has undergone a kidney transplant, the method comprising: a) determining the expression level of at least two of three biomarkers in microvesicular RNA isolated from a biological sample from the subject, wherein the three biomarkers comprise IL32, B2M, and CXCL11; and b) determining the risk of a kidney transplant rejection in the subject based on the expression levels from step (a).

In some aspects, the present disclosure provides a method of determining that a subject who has undergone kidney transplant is undergoing a transplant rejection, the method comprising: a) determining the expression level of at least two of three biomarkers and at least one endogenous control gene in microvesicular RNA isolated from a biological sample from the subject, wherein the three biomarkers comprise IL32, B2M, and CXCL11; b) normalizing the expression of the biomarkers to the expression level of the at least one endogenous control gene; c) inputting the normalized expression levels from step (b) into an algorithm to generate a score; and d) determining that the subject is undergoing kidney transplant rejection based on the score.

The present disclosure provides a method of determining that a subject who has undergone kidney transplant is undergoing a transplant rejection, the method comprising: a) determining the expression level of at least two of three biomarkers and at least one endogenous control gene in microvesicular RNA isolated from a biological sample from the subject, wherein the three biomarkers comprise IL32, B2M, and CXCL11; b) normalizing the expression of the biomarkers to the expression level of the at least one endogenous control gene; and c) determining that the subject is undergoing kidney transplant rejection based on the normalized expression levels from step (b).

The present disclosure provides a method of determining that a subject who has undergone kidney transplant is undergoing a transplant rejection, the method comprising: a) determining the expression level of at least two of three biomarkers in microvesicular RNA isolated from a biological sample from the subject, wherein the three biomarkers comprise IL32, B2M, and CXCL11; b) inputting the expression levels from step (a) into an algorithm to generate a score; and c) determining that the subject is undergoing kidney transplant rejection based on the score.

The present disclosure provides a method of determining that a subject who has undergone kidney transplant is undergoing a transplant rejection, the method comprising: a) determining the expression level of at least two of three biomarkers in microvesicular RNA isolated from a biological sample from the subject, wherein the three biomarkers comprise IL32, B2M, and CXCL11; and b) determining that the subject is undergoing kidney transplant rejection based on the expression levels from step (a).

In some aspects of the preceding methods, step (a) can further comprise determining the expression level of the biomarkers in cell-free DNA in addition to microvesicular RNA, and the subsequent steps of the method can incorporate the analysis of said cell-free DNA. Thus, in a non-limiting example, the present disclosure provides a method of determining the risk of a kidney transplant rejection in a subject who has undergone a kidney transplant, the method comprising: a) determining the expression level of at least two of three biomarkers and at least one endogenous control gene in microvesicular RNA and cell-free DNA isolated from a biological sample from the subject, wherein the three biomarkers comprise IL32, B2M, and CXCL11; b) normalizing the expression of the biomarkers to the expression level of the at least one endogenous control gene; c) inputting the normalized expression levels from step (b) into an algorithm to generate a score; and d) determining the risk of a kidney transplant rejection in the subject based on the score.

In some aspects of the preceding methods, the subject is a subject who has been identified to be at risk for a kidney transplant rejection based on at least one clinical indications of kidney transplant rejection.

In some aspects of the preceding methods, the at least one clinical indication of kidney transplant rejection can comprise increased serum creatine.

In some aspects of the preceding methods, step (a) can comprise determining the expression level of each of the three biomarkers.

In some aspects of the preceding methods, determining the risk of a kidney transplant rejection in the subject based on the score can comprise: i) comparing the score to a predetermined cutoff value; and ii) determining that the at the subject is at a high risk of having a kidney transplant rejection when the score is greater than or equal to the predetermined cutoff value or determining that the subject is at low risk of having a kidney transplant rejection when the score is less than the predetermined cutoff value.

In some aspects, determining the risk of a kidney transplant rejection in a subject based on expression levels, normalized expressions levels or a score can comprise comparing the expression levels, normalized expression levels or a score to corresponding predetermined cutoff values and determining the risk of kidney transplant rejection based on the relationship between the expression levels, the normalized expression levels, and the score and the corresponding predetermined cutoff values (e.g. greater than, greater than or equal to, less than, less than or equal to, or equal to).

In some aspects, determining that the subject is undergoing kidney transplant rejection.

based on expression levels, normalized expressions levels or a score can comprise comparing the expression levels, normalized expression levels or a score to corresponding predetermined cutoff values and determining that the subject is undergoing kidney transplant rejection based on the relationship between the expression levels, the normalized expression levels, or the score and the corresponding predetermined cutoff values (e.g. greater than, greater than or equal to, less than, less than or equal to, or equal to).

In some aspects of the preceding methods, the algorithm can be the product of a trained classifier built from at least one predictive classification algorithm, wherein the at least one predictive classification algorithm comprises SVM-linear.

In some aspects of the preceding methods, a trained classifier can be trained using the expression levels of the biomarkers measured in RNA (e.g. microvesicular RNA, cell-free RNA, etc.) isolated from a training set of biological samples. In some aspects, a training set of biological sample can comprise: i) a first plurality of biological samples isolated from subjects identified to be at risk for a kidney transplant rejection based on at least one clinical indications of kidney transplant rejection. In some aspects, the at least one clinical indication of kidney transplant rejection can comprise increased serum creatine.

In some aspects of the preceding methods, a portion of the biological samples in the first plurality of biological samples in the training set can be from subjects who are identified by biopsy to be positive for kidney transplant rejection and a portion of the biological samples in the first plurality of biological samples in the training set can be from subjects who are identified by biopsy to be negative for kidney transplant rejection.

In some aspects of the preceding methods, a training set of biological samples can further comprise a second plurality of biological samples isolated from subjects that have no clinical indications of a kidney transplant rejection. In some aspects, this second plurality of biological samples can be from subjects identified by biopsy to be negative for kidney transplant rejection.

In some aspects of the preceding methods, an algorithm and a corresponding predetermined cutoff value can have, or be selected to have, a sensitivity for identifying kidney transplant rejection of at least about 90%. In some aspects of the preceding methods, an algorithm and a corresponding predetermined cutoff value can have, or be selected to have, a sensitivity for identifying kidney transplant rejection of at least about 93%.

In some aspects of the preceding methods, an algorithm and a corresponding predetermined cutoff value can have, or be selected to have, a specificity for identifying kidney transplant rejection of at least about 40%. In some aspects of the preceding methods, an algorithm and a corresponding predetermined cutoff value can have, or be selected to have, a specificity for identifying kidney transplant rejection of at least about 43%.

In some aspects of the preceding methods, an algorithm and a corresponding predetermined cutoff value can have, or be selected to have, a positive predictive value (PPV) for identifying kidney transplant rejection of at least about 40%. In some aspects of the preceding methods, an algorithm and a corresponding predetermined cutoff value can have, or be selected to have, a PPV for identifying kidney transplant rejection of at least about 45%.

In some aspects of the preceding methods, an algorithm and a corresponding predetermined cutoff value can have, or be selected to have, a negative predictive value (NPV) for identifying kidney transplant rejection of at least about 90%. In some aspects of the preceding methods, an algorithm and a corresponding predetermined cutoff value can have, or be selected to have, a NPV for identifying kidney transplant rejection of at least about 93%.

### Classifier #2

The present disclosure provides a method of determining the risk of kidney inflammation in a subject, the method comprising: a) determining the expression level of at least two of three biomarkers and at least one endogenous control gene in RNA isolated from a biological sample from the subject, wherein the three biomarkers comprise IL32, B2M, and CXCL11; b) normalizing the expression of the biomarkers to the expression level of the at least one endogenous control gene; c) inputting the normalized expression levels from step (b) into an algorithm to generate a score; and d) determining the risk of a kidney inflammation in the subject based on the score.

The present disclosure provides a method of determining the risk of kidney inflammation in a subject, the method comprising: a) determining the expression level of at least two of three biomarkers and at least one endogenous control gene in RNA isolated from a biological sample from the subject, wherein the three biomarkers comprise IL32, B2M, and CXCL11; b) normalizing the expression of the biomarkers to the expression level of the at least one endogenous control gene; and c) determining the risk of a kidney inflammation in the subject based on the normalized expression levels from step (b).

The present disclosure provides a method of determining the risk of kidney inflammation in a subject, the method comprising: a) determining the expression level of at least two of three biomarkers in RNA isolated from a biological sample from the subject, wherein the three biomarkers comprise IL32, B2M, and CXCL11; b) inputting the expression levels from step (a) into an algorithm to generate a score; and c) determining the risk of a kidney inflammation in the subject based on the score.

The present disclosure provides a method of determining the risk of kidney inflammation in a subject, the method comprising: a) determining the expression level of at least two of three biomarkers in RNA isolated from a biological sample from the subject, wherein the three biomarkers comprise IL32, B2M, and CXCL11; and b) determining the risk of a kidney inflammation in the subject based on the expression levels from step (a).

In some aspects, the present disclosure provides a method of determining that a subject has kidney inflammation, the method comprising: a) determining the expression level of at least two of three biomarkers and at least one endogenous control gene in RNA isolated from a biological sample from the subject, wherein the three biomarkers comprise IL32, B2M, and CXCL11; b) normalizing the expression of the biomarkers to the expression level of the at least one endogenous control gene; c) inputting the normalized expression levels from step (b) into an algorithm to generate a score; and d) determining that the subject has kidney inflammation based on the score.

The present disclosure provides a method of determining that a subject has kidney inflammation, the method comprising: a) determining the expression level of at least two of three biomarkers and at least one endogenous control gene in RNA isolated from a biological sample from the subject, wherein the three biomarkers comprise IL32, B2M, and CXCL11; b) normalizing the expression of the biomarkers to the expression level of the at least one endogenous control gene; and c) determining that the subject has kidney inflammation based on the normalized expression levels from step (b).

The present disclosure provides a method of determining that a subject has kidney inflammation, the method comprising: a) determining the expression level of at least two of three biomarkers in RNA isolated from a biological sample from the subject, wherein the three biomarkers comprise IL32, B2M, and CXCL11; b) inputting the expression levels from step (a) into an algorithm to generate a score; and c) determining that the subject has kidney inflammation on the score.

The present disclosure provides a method of determining that a subject has kidney inflammation, the method comprising: a) determining the expression level of at least two of three biomarkers in RNA isolated from a biological sample from the subject, wherein the three biomarkers comprise IL32, B2M, and CXCL11; and b) determining that the subject has kidney inflammation based on the expression levels from step (a).

In some aspects, the RNA isolated from a biological sample from the subject can comprise cell-free RNA. In some aspects, the RNA isolated from a biological sample from the subject can comprise microvesicular RNA.

Accordingly, the present disclosure provides a method of determining the risk of kidney inflammation in a subject, the method comprising: a) determining the expression level of at least two of three biomarkers and at least one endogenous control gene in microvesicular RNA isolated from a biological sample from the subject, wherein the three biomarkers comprise IL32, B2M, and CXCL11; b) normalizing the expression of the biomarkers to the expression level of the at least one endogenous control gene; c) inputting the normalized expression levels from step (b) into an algorithm to generate a score; and d) determining the risk of a kidney inflammation in the subject based on the score.

The present disclosure provides a method of determining the risk of kidney inflammation in a subject, the method comprising: a) determining the expression level of at least two of three biomarkers and at least one endogenous control gene in microvesicular RNA isolated from a biological sample from the subject, wherein the three biomarkers comprise IL32, B2M, and CXCL11; b) normalizing the expression of the biomarkers to the expression level of the at least one endogenous control gene; and c) determining the risk of a kidney inflammation in the subject based on the normalized expression levels from step (b).

The present disclosure provides a method of determining the risk of kidney inflammation in a subject, the method comprising: a) determining the expression level of at least two of three biomarkers in microvesicular RNA isolated from a biological sample from the subject, wherein the three biomarkers comprise IL32, B2M, and CXCL11; b) inputting the expression levels from step (a) into an algorithm to generate a score; and c) determining the risk of a kidney inflammation in the subject based on the score.

The present disclosure provides a method of determining the risk of kidney inflammation in a subject, the method comprising: a) determining the expression level of at least two of three biomarkers in microvesicular RNA isolated from a biological sample from the subject, wherein the three biomarkers comprise IL32, B2M, and CXCL11; and b) determining the risk of a kidney inflammation in the subject based on the expression levels from step (a).

In some aspects, the present disclosure provides a method of determining that a subject has kidney inflammation, the method comprising: a) determining the expression level of at least two of three biomarkers and at least one endogenous control gene in microvesicular RNA isolated from a biological sample from the subject, wherein the three biomarkers comprise IL32, B2M, and CXCL11; b) normalizing the expression of the biomarkers to the expression level of the at least one endogenous control gene; c) inputting the normalized expression levels from step (b) into an algorithm to generate a score; and d) determining that the subject has kidney inflammation based on the score.

The present disclosure provides a method of determining that a subject has kidney inflammation, the method comprising: a) determining the expression level of at least two of three biomarkers and at least one endogenous control gene in microvesicular RNA isolated from a biological sample from the subject, wherein the three biomarkers comprise IL32, B2M, and CXCL11; b) normalizing the expression of the biomarkers to the expression level of the at least one endogenous control gene; and c) determining that the subject has kidney inflammation based on the normalized expression levels from step (b).

The present disclosure provides a method of determining that a subject has kidney inflammation, the method comprising: a) determining the expression level of at least two of three biomarkers in microvesicular RNA isolated from a biological sample from the subject, wherein the three biomarkers comprise IL32, B2M, and CXCL11; b) inputting the expression levels from step (a) into an algorithm to generate a score; and c) determining that the subject has kidney inflammation based on the score.

The present disclosure provides a method of determining that a subject has kidney inflammation, the method comprising: a) determining the expression level of at least two of three biomarkers in microvesicular RNA isolated from a biological sample from the subject, wherein the three biomarkers comprise IL32, B2M, and CXCL11; and b) determining that the subject has kidney inflammation based on the expression levels from step (a).

In some aspects of the preceding methods, step (a) can further comprise determining the expression level of the biomarkers in cell-free DNA in addition to microvesicular RNA, and the subsequent steps of the method can incorporate the analysis of said cell-free DNA. Thus, in a non-limiting example, the present disclosure provides a method of determining the risk of kidney inflammation in a subject, the method comprising: a) determining the expression level of at least two of three biomarkers and at least one endogenous control gene in microvesicular RNA and cell-free DNA isolated from a biological sample from the subject, wherein the three biomarkers comprise IL32, B2M, and CXCL11; b) normalizing the expression of the biomarkers to the expression level of the at least one endogenous control gene; c) inputting the normalized expression levels from step (b) into an algorithm to generate a score; and d) determining the risk of a kidney inflammation in the subject based on the score.

In some aspects of the preceding methods, the subject is a subject who has undergone a kidney transplant. In some aspects of the preceding methods, the subject is a subject who has not undergone a kidney transplant.

In some aspects of the preceding methods, the subject is a subject who has been identified to be at risk of kidney inflammation.

In some aspects of the preceding methods, the subject is a subject who has been identified to be at risk of kidney inflammation based on at least one clinical indications of kidney inflammation.

In some aspects of the preceding methods, the subject is a subject who has been identified to be at risk of kidney inflammation has undergone a kidney transplant.

In some aspects of the preceding methods, the subject is a subject who has been identified to be at risk for a kidney transplant rejection based on at least one clinical indications of kidney transplant rejection.

In some aspects of the preceding methods, the subject is a subject who has been identified to be at risk for a kidney transplant rejection based on at least one clinical indications of kidney transplant rejection.

In some aspects of the preceding methods, the at least one clinical indication of kidney transplant rejection can comprise increased serum creatine.

In some aspects of the preceding methods, the kidney inflammation may be caused by BKV infection, recurrent glomerulonephritis, urinary tract inflammation, BK viremia, CMV viremia, another inflammatory condition that affects the kidney or any combination thereof.

In some aspects, the kidney inflammation can be pathological lymphoproliferative infiltrate, moderate to severe lymphocytic infiltration, interstitial nephritis, glomerulopoathy, immune complex deposition, or any combination thereof, in some aspects, interstitial nephritis can be BKV nephritis or acute interstitial nephritis.

In some aspects of the preceding methods, step (a) can comprise determining the expression level of each of the three biomarkers.

In some aspects of the preceding methods, determining the risk of kidney inflammation in the subject based on the score can comprise: i) comparing the score to a predetermined cutoff value; and ii) determining that the at the subject is at a high risk of having kidney inflammation when the score is greater than or equal to the predetermined cutoff value or determining that the subject is at low risk of having a kidney inflammation when the score is less than the predetermined cutoff value.

In some aspects, determining the risk of a kidney inflammation in a subject based on expression levels, normalized expressions levels or a score can comprise comparing the expression levels, normalized expression levels or a score to corresponding predetermined cutoff values and determining the risk of kidney inflammation based on the relationship between the expression levels, the normalized expression levels, and the score and the corresponding predetermined cutoff values (e.g. greater than, greater than or equal to, less than, less than or equal to, or equal to).

In some aspects, determining that the subject has kidney inflammation based on expression levels, normalized expressions levels or a score can comprise comparing the expression levels, normalized expression levels or a score to corresponding predetermined cutoff values and determining that the subject has kidney inflammation based on the relationship between the expression levels, the normalized expression levels, or the score and the corresponding predetermined cutoff values (e.g. greater than, greater than or equal to, less than, less than or equal to, or equal to).

In some aspects of the preceding methods, the algorithm can be the product of a trained classifier built from at least one predictive classification algorithm, wherein the at least one predictive classification algorithm comprises SVM-linear.

In some aspects of the preceding methods, a trained classifier can be trained using the expression levels of the biomarkers measured in RNA (e.g. microvesicular RNA, cell-free RNA, etc.) isolated from a training set of biological samples. In some aspects, a training set of biological sample can comprise: i) a first plurality of biological samples isolated from subjects identified to be at risk for a kidney transplant rejection based on at least one clinical indications of kidney transplant rejection. In some aspects, the at least one clinical indication of kidney transplant rejection can comprise increased serum creatine.

In some aspects of the preceding methods, a portion of the biological samples in the first plurality of biological samples in the training set can be from subjects who are identified by biopsy to be positive for kidney transplant rejection and a portion of the biological samples in the first plurality of biological samples in the training set can be from subjects who are identified by biopsy to be negative for kidney transplant rejection.

In some aspects of the preceding methods, a training set of biological samples can further comprise a second plurality of biological samples isolated from subjects that have no clinical indications of a kidney transplant rejection. In some aspects, this second plurality of biological samples can be from subjects identified by biopsy to be negative for kidney transplant rejection.

In some aspects of the preceding methods, an algorithm and a corresponding predetermined cutoff value can have, or be selected to have, a sensitivity for identifying kidney inflammation of at least about 90%. In some aspects of the preceding methods, an algorithm and a corresponding predetermined cutoff value can have, or be selected to have, a sensitivity for identifying kidney inflammation of at least about 94%.

In some aspects of the preceding methods, an algorithm and a corresponding predetermined cutoff value can have, or be selected to have, a specificity for identifying kidney inflammation of at least about 50%. In some aspects of the preceding methods, an algorithm and a corresponding predetermined cutoff value can have, or be selected to have, a specificity for identifying kidney inflammation of at least about 52%.

In some aspects of the preceding methods, an algorithm and a corresponding predetermined cutoff value can have, or be selected to have, a positive predictive value (PPV) for identifying kidney inflammation of at least about 60%. In some aspects of the preceding methods, an algorithm and a corresponding predetermined cutoff value can have, or be selected to have, a PPV for identifying kidney inflammation of at least about 62%.

In some aspects of the preceding methods, an algorithm and a corresponding predetermined cutoff value can have, or be selected to have, a negative predictive value (NPV) for identifying kidney inflammation of at least about 90%. In some aspects of the preceding methods, an algorithm and a corresponding predetermined cutoff value can have, or be selected to have, a NPV for identifying kidney inflammation of at least about 91%.

### Classifier #3

The present disclosure provides a method of determining the risk of a kidney transplant rejection in a subject who has undergone a kidney transplant, the method comprising: a) determining the expression level of at least two of six biomarkers and at least one endogenous control gene in RNA isolated from a biological sample from the subject, wherein the six biomarkers comprise IFNAR2, PYCARD, CD44, IRAK2, B2M, and NAMPT; b) normalizing the expression of the biomarkers to the expression level of the at least one endogenous control gene; c) inputting the normalized expression levels from step (b) into an algorithm to generate a score; and d) determining the risk of a kidney transplant rejection in the subject based on the score.

The present disclosure provides a method of determining the risk of a kidney transplant rejection in a subject who has undergone a kidney transplant, the method comprising: a) determining the expression level of at least two of six biomarkers and at least one endogenous control gene in RNA isolated from a biological sample from the subject, wherein the six biomarkers comprise IFNAR2, PYCARD, CD44, IRAK2, B2M, and NAMPT; b) normalizing the expression of the biomarkers to the expression level of the at least one endogenous control gene; and c) determining the risk of a kidney transplant rejection in the subject based on the normalized expression levels from step (b).

The present disclosure provides a method of determining the risk of a kidney transplant rejection in a subject who has undergone a kidney transplant, the method comprising: a) determining the expression level of at least two of six biomarkers in RNA isolated from a biological sample from the subject, wherein the six biomarkers comprise IFNAR2, PYCARD, CD44, IRAK2, B2M, and NAMPT; b) inputting the expression levels from step (a) into an algorithm to generate a score; and c) determining the risk of a kidney transplant rejection in the subject based on the score.

The present disclosure provides a method of determining the risk of a kidney transplant rejection in a subject who has undergone a kidney transplant, the method comprising: a) determining the expression level of at least two of six biomarkers in RNA isolated from a biological sample from the subject, wherein the six biomarkers comprise IFNAR2, PYCARD, CD44, IRAK2, B2M, and NAMPT; and b) determining the risk of a kidney transplant rejection in the subject based on the expression levels from step (a).

In some aspects, the present disclosure provides a method of determining that a subject who has undergone kidney transplant is not undergoing a transplant rejection, the method comprising: a) determining the expression level of at least two of six biomarkers and at least one endogenous control gene in RNA isolated from a biological sample from the subject, wherein the six biomarkers comprise IFNAR2, PYCARD, CD44, IRAK2, B2M, and NAMPT; b) normalizing the expression of the biomarkers to the expression level of the at least one endogenous control gene; c) inputting the normalized expression levels from step (b) into an algorithm to generate a score; and d) determining that the subject is not undergoing kidney transplant rejection based on the score.

The present disclosure provides a method of determining that a subject who has undergone kidney transplant is not undergoing a transplant rejection, the method comprising: a) determining the expression level of at least two of six biomarkers and at least one endogenous control gene in RNA isolated from a biological sample from the subject, wherein the six biomarkers comprise IFNAR2, PYCARD, CD44, IRAK2, B2M, and NAMPT; b) normalizing the expression of the biomarkers to the expression level of the at least one endogenous control gene; and c) determining that the subject is not undergoing kidney transplant rejection based on the normalized expression levels from step (b).

The present disclosure provides a method of determining that a subject who has undergone kidney transplant is not undergoing a transplant rejection, the method comprising: a) determining the expression level of at least two of six biomarkers in RNA isolated from a biological sample from the subject, wherein the six biomarkers comprise IFNAR2, PYCARD, CD44, IRAK2, B2M, and NAMPT; b) inputting the expression levels from step (a) into an algorithm to generate a score; and c) determining that the subject is not undergoing kidney transplant rejection based on the score.

The present disclosure provides a method of determining that a subject who has undergone kidney transplant is not undergoing a transplant rejection, the method comprising: a) determining the expression level of at least two of six biomarkers in RNA isolated from a biological sample from the subject, wherein the six biomarkers comprise IFNAR2, PYCARD, CD44, IRAK2, B2M, and NAMPT; and b) determining that the subject is not undergoing kidney transplant rejection based on the expression levels from step (a).

In some aspects, the RNA isolated from a biological sample from the subject can comprise cell-free RNA. In some aspects, the RNA isolated from a biological sample from the subject can comprise microvesicular RNA.

Accordingly, the present disclosure provides a method of determining the risk of a kidney transplant rejection in a subject who has undergone a kidney transplant, the method comprising: a) determining the expression level of at least two of six biomarkers and at least one endogenous control gene in microvesicular RNA isolated from a biological sample from the subject, wherein the six biomarkers comprise IFNAR2, PYCARD, CD44, IRAK2, B2M, and NAMPT; b) normalizing the expression of the biomarkers to the expression level of the at least one endogenous control gene; c) inputting the normalized expression levels from step (b) into an algorithm to generate a score; and d) determining the risk of a kidney transplant rejection in the subject based on the score.

The present disclosure provides a method of determining the risk of a kidney transplant rejection in a subject who has undergone a kidney transplant, the method comprising: a) determining the expression level of at least two of six biomarkers and at least one endogenous control gene in microvesicular RNA isolated from a biological sample from the subject, wherein the six biomarkers comprise IFNAR2, PYCARD, CD44, IRAK2, B2M, and NAMPT; b) normalizing the expression of the biomarkers to the expression level of the at least one endogenous control gene; and c) determining the risk of a kidney transplant rejection in the subject based on the normalized expression levels from step (b).

The present disclosure provides a method of determining the risk of a kidney transplant rejection in a subject who has undergone a kidney transplant, the method comprising: a) determining the expression level of at least two of six biomarkers in microvesicular RNA isolated from a biological sample from the subject, wherein the six biomarkers comprise IFNAR2, PYCARD, CD44, IRAK2, B2M, and NAMPT; b) inputting the expression levels from step (a) into an algorithm to generate a score; and c) determining the risk of a kidney transplant rejection in the subject based on the score.

The present disclosure provides a method of determining the risk of a kidney transplant rejection in a subject who has undergone a kidney transplant, the method comprising: a) determining the expression level of at least two of six biomarkers in microvesicular RNA isolated from a biological sample from the subject, wherein the six biomarkers comprise IFNAR2, PYCARD, CD44, IRAK2, B2M, and NAMPT; and b) determining the risk of a kidney transplant rejection in the subject based on the expression levels from step (a).

In some aspects, the present disclosure provides a method of determining that a subject who has undergone kidney transplant is not undergoing a transplant rejection, the method comprising: a) determining the expression level of at least two of six biomarkers and at least one endogenous control gene in microvesicular RNA isolated from a biological sample from the subject, wherein the six biomarkers comprise IFNAR2, PYCARD, CD44, IRAK2, B2M, and NAMPT; b) normalizing the expression of the biomarkers to the expression level of the at least one endogenous control gene; c) inputting the normalized expression levels from step (b) into an algorithm to generate a score; and d) determining that the subject is not undergoing kidney transplant rejection based on the score.

The present disclosure provides a method of determining that a subject who has undergone kidney transplant is not undergoing a transplant rejection, the method comprising: a) determining the expression level of at least two of six biomarkers and at least one endogenous control gene in microvesicular RNA isolated from a biological sample from the subject, wherein the six biomarkers comprise IFNAR2, PYCARD, CD44, IRAK2, B2M, and NAMPT; b) normalizing the expression of the biomarkers to the expression level of the at least one endogenous control gene; and c) determining that the subject is not undergoing kidney transplant rejection based on the normalized expression levels from step (b).

The present disclosure provides a method of determining that a subject who has undergone kidney transplant is not undergoing a transplant rejection, the method comprising: a) determining the expression level of at least two of six biomarkers in microvesicular RNA isolated from a biological sample from the subject, wherein the six biomarkers comprise IFNAR2, PYCARD, CD44, IRAK2, B2M, and NAMPT; b) inputting the expression levels from step (a) into an algorithm to generate a score; and c) determining that the subject is not undergoing kidney transplant rejection based on the score.

The present disclosure provides a method of determining that a subject who has undergone kidney transplant is not undergoing a transplant rejection, the method comprising: a) determining the expression level of at least two of six biomarkers in microvesicular RNA isolated from a biological sample from the subject, wherein the six biomarkers comprise IFNAR2, PYCARD, CD44, IRAK2, B2M, and NAMPT; and b) determining that the subject is not undergoing kidney transplant rejection based on the expression levels from step (a).

In some aspects of the preceding methods, step (a) can further comprise determining the expression level of the biomarkers in cell-free DNA in addition to microvesicular RNA, and the subsequent steps of the method can incorporate the analysis of said cell-free DNA. Thus, in a non-limiting example, the present disclosure provides a method of determining the risk of a kidney transplant rejection in a subject who has undergone a kidney transplant, the method comprising: a) determining the expression level of at least two of six biomarkers and at least one endogenous control gene in microvesicular RNA and cell-free DNA isolated from a biological sample from the subject, wherein the six biomarkers comprise IFNAR2, PYCARD, CD44, IRAK2, B2M, and NAMPT; b) normalizing the expression of the biomarkers to the expression level of the at least one endogenous control gene; c) inputting the normalized expression levels from step (b) into an algorithm to generate a score; and d) determining the risk of a kidney transplant rejection in the subject based on the score.

In some aspects of the preceding methods, the subject is a subject that has no clinical indications of a kidney transplant rejection.

In some aspects of the preceding methods, step (a) can comprise determining the expression level of at least three, or at least four, or at least five of the six biomarkers.

In some aspects of the preceding methods, step (a) can comprise determining the expression level of:
i) IFNAR2, PYCARD;
ii) IFNAR2, CD44;
iii) IFNAR2, IRAK2;
iv) IFNAR2, B2M;
v) IFNAR2, NAMPT;
vi) PYCARD, CD44;
vii) PYCARD, IRAK2;
viii) PYCARD, B2M;
ix) PYCARD, NAMPT;
x) CD44, IRAK2;
xi) CD44, B2M;
xii) CD44, NAMPT;
xiii) IRAK2, B2M;
xiv) IRAK2, NAMPT; or
xv) B2M, NAMPT.

In some aspects of the preceding methods, step (a) can comprise determining the expression level of:
i) IFNAR2, PYCARD, CD44;
ii) IFNAR2, PYCARD, IRAK2;
iii) IFNAR2, PYCARD, B2M;
iv) IFNAR2, PYCARD, NAMPT;
v) IFNAR2, CD44, IRAK2;
vi) IFNAR2, CD44, B2M;
vii) IFNAR2, CD44, NAMPT;
viii) IFNAR2, IRAK2, B2M;
ix) IFNAR2, IRAK2, NAMPT;
x) IFNAR2, B2M, NAMPT;
xi) PYCARD, CD44, IRAK2;
xii) PYCARD, CD44, B2M;
xiii) PYCARD, CD44, NAMPT;
xiv) PYCARD, IRAK2, B2M;
xv) PYCARD, IRAK2, NAMPT;
xvi) PYCARD, B2M, NAMPT;
xvii) CD44, IRAK2, B2M;
xviii) CD44, IRAK2, NAMPT;
xix) CD44, B2M, NAMPT; or
xx) IRAK2, B2M, NAMPT.

In some aspects of the preceding methods, step (a) can comprise determining the expression level of:
i) IFNAR2, PYCARD, CD44, IRAK2;
ii) IFNAR2, PYCARD, CD44, B2M;
iii) IFNAR2, PYCARD, CD44, NAMPT;
iv) IFNAR2, PYCARD, IRAK2, B2M;
v) IFNAR2, PYCARD, IRAK2, NAMPT;
vi) IFNAR2, PYCARD, B2M, NAMPT;
vii) IFNAR2, CD44, IRAK2, B2M;
viii) IFNAR2, CD44, IRAK2, NAMPT;
ix) IFNAR2, CD44, B2M, NAMPT;
x) IFNAR2, IRAK2, B2M, NAMPT;
xi) PYCARD, CD44, IRAK2, B2M;
xii) PYCARD, CD44, IRAK2, NAMPT;
xiii) PYCARD, CD44, B2M, NAMPT;
xiv) PYCARD, IRAK2, B2M, NAMPT; or
xv) CD44, IRAK2, B2M, NAMPT.

In some aspects of the preceding methods, step (a) can comprise determining the expression level of:
i) IFNAR2, PYCARD, CD44, IRAK2, B2M;
ii) IFNAR2, PYCARD, CD44, IRAK2, NAMPT;
iii) IFNAR2, PYCARD, CD44, B2M, NAMPT;
iv) IFNAR2, PYCARD, IRAK2, B2M, NAMPT;
v) IFNAR2, CD44, IRAK2, B2M, NAMPT; or
vi) PYCARD, CD44, IRAK2, B2M, NAMPT.

In some aspects of the preceding methods, step (a) can comprise determining the expression level of each of the six biomarkers.

In some aspects of the preceding methods, determining the risk of a kidney transplant rejection in the subject based on the score can comprise: i) comparing the score to a predetermined cutoff value; and ii) determining that the at the subject is at a high risk of having a kidney transplant rejection when the score is greater than or equal to the predetermined cutoff value or determining that the subject is at low risk of having a kidney transplant rejection when the score is less than the predetermined cutoff value.

In some aspects, determining the risk of a kidney transplant rejection in a subject based on expression levels, normalized expressions levels or a score can comprise comparing the expression levels, normalized expression levels or a score to corresponding predetermined cutoff values and determining the risk of kidney transplant rejection based on the relationship between the expression levels, the normalized expression levels, and the score and the corresponding predetermined cutoff values (e.g. greater than, greater than or equal to, less than, less than or equal to, or equal to).

In some aspects, determining that the subject is not undergoing kidney transplant rejection based on expression levels, normalized expressions levels or a score can comprise comparing the expression levels, normalized expression levels or a score to corresponding predetermined cutoff values and determining that the subject is not undergoing kidney transplant rejection based on the relationship between the expression levels, the normalized expression levels, or the score and the corresponding predetermined cutoff values (e.g. greater than, greater than or equal to, less than, less than or equal to, or equal to).

In some aspects of the preceding methods, the algorithm can be the product of: a first trained classifier built from at least one predictive classification algorithm, wherein the at least one predictive classification algorithm comprises naïve Bayes; and an at least second trained classifier built from at least one predictive classification algorithm, wherein the at least one predictive classification algorithm comprises naïve Bayes.

In some aspects, the first trained classifier can comprise two bivariate features. In some aspects, the two bivariate features are IFNAR2|PYCARD and CD44|IRAK2.

In some aspects, the second trained classifier can comprise two univariate features. In some aspects, the two univariate features are B2M and NAMPT.

In some aspects of the preceding methods, the first and/or the at least second trained classifier(s) can be trained using the expression levels of the biomarkers measured in RNA (e.g. microvesicular RNA, cell-free RNA, etc.) isolated from a training set of biological samples. In some aspects, a training set of biological samples can comprise: i) a first plurality of biological samples isolated from subjects that have no clinical indications of a kidney transplant rejection.

In some aspects of the preceding methods, a portion of the biological samples in the first plurality of biological samples in the training set can be from subjects who are identified by biopsy to be positive for kidney transplant rejection and a portion of the biological samples in the first plurality of biological samples in the training set can be from subjects who are identified by biopsy to be negative for kidney transplant rejection.

In some aspects of the preceding methods, a training set of biological samples can further comprise a second plurality of biological samples isolated from subjects identified to be at risk for a kidney transplant rejection based on at least one clinical indications of kidney transplant rejection. In some aspects, this second plurality of biological samples can be from subjects identified by biopsy to be positive for kidney transplant rejection.

In some aspects of the preceding methods, an algorithm and a corresponding predetermined cutoff value can have, or be selected to have, a sensitivity for identifying kidney transplant rejection of at least about 90%. In some aspects of the preceding methods, an algorithm and a corresponding predetermined cutoff value can have, or be selected to have, a sensitivity for identifying kidney transplant rejection of at least about 93%.

In some aspects of the preceding methods, an algorithm and a corresponding predetermined cutoff value can have, or be selected to have, a specificity for identifying kidney transplant rejection of at least about 40%. In some aspects of the preceding methods, an algorithm and a corresponding predetermined cutoff value can have, or be selected to have, a specificity for identifying kidney transplant rejection of at least about 48%.

In some aspects of the preceding methods, an algorithm and a corresponding predetermined cutoff value can have, or be selected to have, a positive predictive value (PPV) for identifying kidney transplant rejection of at least about 30%. In some aspects of the preceding methods, an algorithm and a corresponding predetermined cutoff value can have, or be selected to have, a PPV for identifying kidney transplant rejection of at least about 32%.

In some aspects of the preceding methods, an algorithm and a corresponding predetermined cutoff value can have, or be selected to have, a negative predictive value (NPV) for identifying kidney transplant rejection of at least about 90%. In some aspects of the preceding methods, an algorithm and a corresponding predetermined cutoff value can have, or be selected to have, a NPV for identifying kidney transplant rejection of at least about 97%.

In some aspects of the preceding methods, an algorithm and a corresponding predetermined cutoff value can have, or be selected to have, a sensitivity for identifying kidney transplant rejection of at least about 60%. In some aspects of the preceding methods, an algorithm and a corresponding predetermined cutoff value can have, or be selected to have, a sensitivity for identifying kidney transplant rejection of at least about 61%.

In some aspects of the preceding methods, an algorithm and a corresponding predetermined cutoff value can have, or be selected to have, a specificity for identifying kidney transplant rejection of at least about 80%. In some aspects of the preceding methods, an algorithm and a corresponding predetermined cutoff value can have, or be selected to have, a specificity for identifying kidney transplant rejection of at least about 84%.

In some aspects of the preceding methods, an algorithm and a corresponding predetermined cutoff value can have, or be selected to have, a positive predictive value (PPV) for identifying kidney transplant rejection of at least about 50%.

In some aspects of the preceding methods, an algorithm and a corresponding predetermined cutoff value can have, or be selected to have, a negative predictive value (NPV) for identifying kidney transplant rejection of at least about 90%.

### Classifier #4

The present disclosure provides a method of determining the risk of a kidney transplant rejection in a subject who has undergone a kidney transplant, the method comprising: a) determining the expression level of at least two of four biomarkers and at least one endogenous control gene in RNA isolated from a biological sample from the subject, wherein the four biomarkers comprise IFNAR2, PYCARD, CD44, and IRAK2; b) normalizing the expression of the biomarkers to the expression level of the at least one endogenous control gene; c) inputting the normalized expression levels from step (b) into an algorithm to generate a score; and d) determining the risk of a kidney transplant rejection in the subject based on the score.

The present disclosure provides a method of determining the risk of a kidney transplant rejection in a subject who has undergone a kidney transplant, the method comprising: a) determining the expression level of at least two of four biomarkers and at least one endogenous control gene in RNA isolated from a biological sample from the subject, wherein the four biomarkers comprise IFNAR2, PYCARD, CD44, and IRAK2; b) normalizing the expression of the biomarkers to the expression level of the at least one endogenous control gene; and c) determining the risk of a kidney transplant rejection in the subject based on the normalized expression levels from step (b).

The present disclosure provides a method of determining the risk of a kidney transplant rejection in a subject who has undergone a kidney transplant, the method comprising: a) determining the expression level of at least two of four biomarkers in RNA isolated from a biological sample from the subject, wherein the four biomarkers comprise IFNAR2, PYCARD, CD44, and IRAK2; b) inputting the expression levels from step (a) into an algorithm to generate a score; and c) determining the risk of a kidney transplant rejection in the subject based on the score.

The present disclosure provides a method of determining the risk of a kidney transplant rejection in a subject who has undergone a kidney transplant, the method comprising: a) determining the expression level of at least two of four biomarkers in RNA isolated from a biological sample from the subject, wherein the four biomarkers comprise IFNAR2, PYCARD, CD44, and IRAK2; and b) determining the risk of a kidney transplant rejection in the subject based on the expression levels from step (a).

In some aspects, the present disclosure provides a method of determining that a subject who has undergone kidney transplant is not undergoing a transplant rejection, the method comprising: a) determining the expression level of at least two of four biomarkers and at least one endogenous control gene in RNA isolated from a biological sample from the subject, wherein the four biomarkers comprise IFNAR2, PYCARD, CD44, and IRAK2; b) normalizing the expression of the biomarkers to the expression level of the at least one endogenous control gene; c) inputting the normalized expression levels from step (b) into an algorithm to generate a score; and d) determining that the subject is not undergoing kidney transplant rejection based on the score.

The present disclosure provides a method of determining that a subject who has undergone kidney transplant is not undergoing a transplant rejection, the method comprising: a) determining the expression level of at least two of four biomarkers and at least one endogenous control gene in RNA isolated from a biological sample from the subject, wherein the four biomarkers comprise IFNAR2, PYCARD, CD44, and IRAK2; b) normalizing the expression of the biomarkers to the expression level of the at least one endogenous control gene; and c) determining that the subject is not undergoing kidney transplant rejection based on the normalized expression levels from step (b).

The present disclosure provides a method of determining that a subject who has undergone kidney transplant is not undergoing a transplant rejection, the method comprising: a) determining the expression level of at least two of four biomarkers in RNA isolated from a biological sample from the subject, wherein the four biomarkers comprise IFNAR2, PYCARD, CD44, and IRAK2; b) inputting the expression levels from step (a) into an algorithm to generate a score; and c) determining that the subject is not undergoing kidney transplant rejection based on the score.

The present disclosure provides a method of determining that a subject who has undergone kidney transplant is not undergoing a transplant rejection, the method comprising: a) determining the expression level of at least two of four biomarkers in RNA isolated from a biological sample from the subject, wherein the four biomarkers comprise IFNAR2, PYCARD, CD44, and IRAK2; and b) determining that the subject is not undergoing kidney transplant rejection based on the expression levels from step (a).

In some aspects, the RNA isolated from a biological sample from the subject can comprise cell-free RNA. In some aspects, the RNA isolated from a biological sample from the subject can comprise microvesicular RNA.

Accordingly, the present disclosure provides a method of determining the risk of a kidney transplant rejection in a subject who has undergone a kidney transplant, the method comprising: a) determining the expression level of at least two of four biomarkers and at least one endogenous control gene in microvesicular RNA isolated from a biological sample from the subject, wherein the four biomarkers comprise IFNAR2, PYCARD, CD44, and IRAK2; b) normalizing the expression of the biomarkers to the expression level of the at least one endogenous control gene; c) inputting the normalized expression levels from step (b) into an algorithm to generate a score; and d) determining the risk of a kidney transplant rejection in the subject based on the score.

The present disclosure provides a method of determining the risk of a kidney transplant rejection in a subject who has undergone a kidney transplant, the method comprising: a) determining the expression level of at least two of four biomarkers and at least one endogenous control gene in microvesicular RNA isolated from a biological sample from the subject, wherein the four biomarkers comprise IFNAR2, PYCARD, CD44, and IRAK2; b) normalizing the expression of the biomarkers to the expression level of the at least one endogenous control gene; and c) determining the risk of a kidney transplant rejection in the subject based on the normalized expression levels from step (b).

The present disclosure provides a method of determining the risk of a kidney transplant rejection in a subject who has undergone a kidney transplant, the method comprising: a) determining the expression level of at least two of four biomarkers in microvesicular RNA isolated from a biological sample from the subject, wherein the four biomarkers comprise IFNAR2, PYCARD, CD44, and IRAK2; b) inputting the expression levels from step (a) into an algorithm to generate a score; and c) determining the risk of a kidney transplant rejection in the subject based on the score.

The present disclosure provides a method of determining the risk of a kidney transplant rejection in a subject who has undergone a kidney transplant, the method comprising: a) determining the expression level of at least two of four biomarkers in microvesicular RNA isolated from a biological sample from the subject, wherein the four biomarkers comprise IFNAR2, PYCARD, CD44, and IRAK2; and b) determining the risk of a kidney transplant rejection in the subject based on the expression levels from step (a).

In some aspects, the present disclosure provides a method of determining that a subject who has undergone kidney transplant is not undergoing a transplant rejection, the method comprising: a) determining the expression level of at least two of four biomarkers and at least one endogenous control gene in microvesicular RNA isolated from a biological sample from the subject, wherein the four biomarkers comprise IFNAR2, PYCARD, CD44, and IRAK2; b) normalizing the expression of the biomarkers to the expression level of the at least one endogenous control gene; c) inputting the normalized expression levels from step (b) into an algorithm to generate a score; and d) determining that the subject is not undergoing kidney transplant rejection based on the score.

The present disclosure provides a method of determining that a subject who has undergone kidney transplant is not undergoing a transplant rejection, the method comprising: a) determining the expression level of at least two of four biomarkers and at least one endogenous control gene in microvesicular RNA isolated from a biological sample from the subject, wherein the four biomarkers comprise IFNAR2, PYCARD, CD44, and IRAK2; b) normalizing the expression of the biomarkers to the expression level of the at least one endogenous control gene; and c) determining that the subject is not undergoing kidney transplant rejection based on the normalized expression levels from step (b).

The present disclosure provides a method of determining that a subject who has undergone kidney transplant is not undergoing a transplant rejection, the method comprising: a) determining the expression level of at least two of four biomarkers in microvesicular RNA isolated from a biological sample from the subject, wherein the four biomarkers comprise IFNAR2, PYCARD, CD44, and IRAK2; b) inputting the expression levels from step (a) into an algorithm to generate a score; and c) determining that the subject is not undergoing kidney transplant rejection based on the score.

The present disclosure provides a method of determining that a subject who has undergone kidney transplant is not undergoing a transplant rejection, the method comprising: a) determining the expression level of at least two of four biomarkers in microvesicular RNA isolated from a biological sample from the subject, wherein the four biomarkers comprise IFNAR2, PYCARD, CD44, and IRAK2; and b) determining that the subject is not undergoing kidney transplant rejection based on the expression levels from step (a).

In some aspects of the preceding methods, step (a) can further comprise determining the expression level of the biomarkers in cell-free DNA in addition to microvesicular RNA, and the subsequent steps of the method can incorporate the analysis of said cell-free DNA. Thus, in a non-limiting example, the present disclosure provides a method of determining the risk of a kidney transplant rejection in a subject who has undergone a kidney transplant, the method comprising: a) determining the expression level of at least two of four biomarkers and at least one endogenous control gene in RNA isolated from a biological sample from the subject, wherein the four biomarkers comprise IFNAR2, PYCARD, CD44, and IRAK2; b) normalizing the expression of the biomarkers to the expression level of the at least one endogenous control gene; c) inputting the normalized expression levels from step (b) into an algorithm to generate a score; and d) determining the risk of a kidney transplant rejection in the subject based on the score.

In some aspects of the preceding methods, the subject is a subject that has no clinical indications of a kidney transplant rejection.

In some aspects of the preceding methods, step (a) can comprise determining the expression level of at least three of the four biomarkers.

In some aspects of the preceding methods, step (a) can comprise determining the expression level of:
i) IFNAR2, PYCARD;
ii) IFNAR2, CD44;
iii) IFNAR2, IRAK2;
iv) PYCARD, CD44;
v) PYCARD, IRAK2; or
vi) CD44, IRAK2.

In some aspects of the preceding methods, step (a) can comprise determining the expression level of:
i) IFNAR2, PYCARD, CD44;
ii) IFNAR2, PYCARD, IRAK2;
iii) IFNAR2, CD44, IRAK2; or
iv) PYCARD, CD44, IRAK2.

In some aspects of the preceding methods, step (a) can comprise determining the expression level of each of the four biomarkers.

In some aspects of the preceding methods, determining the risk of a kidney transplant rejection in the subject based on the score can comprise: i) comparing the score to a predetermined cutoff value; and ii) determining that the at the subject is at a high risk of having a kidney transplant rejection when the score is greater than or equal to the predetermined cutoff value or determining that the subject is at low risk of having a kidney transplant rejection when the score is less than the predetermined cutoff value.

In some aspects, determining the risk of a kidney transplant rejection in a subject based on expression levels, normalized expressions levels or a score can comprise comparing the expression levels, normalized expression levels or a score to corresponding predetermined cutoff values and determining the risk of kidney transplant rejection based on the relationship between the expression levels, the normalized expression levels, and the score and the corresponding predetermined cutoff values (e.g. greater than, greater than or equal to, less than, less than or equal to, or equal to).

In some aspects, determining that the subject is not undergoing kidney transplant rejection based on expression levels, normalized expressions levels or a score can comprise comparing the expression levels, normalized expression levels or a score to corresponding predetermined cutoff values and determining that the subject is not undergoing kidney transplant rejection based on the relationship between the expression levels, the normalized expression levels, or the score and the corresponding predetermined cutoff values (e.g. greater than, greater than or equal to, less than, less than or equal to, or equal to).

In some aspects of the preceding methods, the algorithm can be the product of: a first trained classifier built from at least one predictive classification algorithm, wherein the at least one predictive classification algorithm comprises naïve Bayes.

In some aspects, the first trained classifier can comprise two bivariate features. In some aspects, the two bivariate features are IFNAR2|PYCARD and CD44|IRAK2.

In some aspects of the preceding methods, the first trained classifier can be trained using the expression levels of the biomarkers measured in RNA (e.g. microvesicular RNA, cell-free RNA, etc.) isolated from a training set of biological samples. In some aspects, a training set of biological samples can comprise: i) a first plurality of biological samples isolated from subjects that have no clinical indications of a kidney transplant rejection.

In some aspects of the preceding methods, a portion of the biological samples in the first plurality of biological samples in the training set can be from subjects who are identified by biopsy to be positive for kidney transplant rejection and a portion of the biological samples in the first plurality of biological samples in the training set can be from subjects who are identified by biopsy to be negative for kidney transplant rejection.

In some aspects of the preceding methods, a training set of biological samples can further comprise a second plurality of biological samples isolated from subjects identified to be at risk for a kidney transplant rejection based on at least one clinical indications of kidney transplant rejection. In some aspects, this second plurality of biological samples can be from subjects identified by biopsy to be positive for kidney transplant rejection.

In some aspects of the preceding methods, an algorithm and a corresponding predetermined cutoff value can have, or be selected to have, a sensitivity for identifying kidney transplant rejection of at least about 90%. In some aspects of the preceding methods, an algorithm and a corresponding predetermined cutoff value can have, or be selected to have, a sensitivity for identifying kidney transplant rejection of at least about 95%.

### Classifier #5

The present disclosure provides a method of determining the risk of a kidney transplant rejection in a subject who has undergone a kidney transplant, the method comprising: a) determining the expression level of two biomarkers and at least one endogenous control gene in RNA isolated from a biological sample from the subject, wherein the two biomarkers comprise B2M and NAMPT; b) normalizing the expression of the biomarkers to the expression level of the at least one endogenous control gene; c) inputting the normalized expression levels from step (b) into an algorithm to generate a score; and d) determining the risk of a kidney transplant rejection in the subject based on the score.

The present disclosure provides a method of determining the risk of a kidney transplant rejection in a subject who has undergone a kidney transplant, the method comprising: a) determining the expression level of two biomarkers and at least one endogenous control gene in RNA isolated from a biological sample from the subject, wherein the two biomarkers comprise B2M and NAMPT; b) normalizing the expression of the biomarkers to the expression level of the at least one endogenous control gene; and c) determining the risk of a kidney transplant rejection in the subject based on the normalized expression levels from step (b).

The present disclosure provides a method of determining the risk of a kidney transplant rejection in a subject who has undergone a kidney transplant, the method comprising: a) determining the expression level of two biomarkers in RNA isolated from a biological sample from the subject, wherein the two biomarkers comprise B2M and NAMPT; b) inputting the expression levels from step (a) into an algorithm to generate a score; and c) determining

The present disclosure provides a method of determining the risk of a kidney transplant rejection in a subject who has undergone a kidney transplant, the method comprising: a) determining the expression level of two biomarkers in RNA isolated from a biological sample from the subject, wherein the two biomarkers comprise B2M and NAMPT; and b) determining the risk of a kidney transplant rejection in the subject based on the expression levels from step (a).

In some aspects, the present disclosure provides a method of determining that a subject who has undergone kidney transplant is undergoing a transplant rejection, the method comprising: a) determining the expression level of two biomarkers and at least one endogenous control gene in RNA isolated from a biological sample from the subject, wherein the two biomarkers comprise B2M and NAMPT; b) normalizing the expression of the biomarkers to the expression level of the at least one endogenous control gene; c) inputting the normalized expression levels from step (b) into an algorithm to generate a score; and d) determining that the subject is undergoing kidney transplant rejection based on the score.

The present disclosure provides a method of determining that a subject who has undergone kidney transplant is undergoing a transplant rejection, the method comprising: a) determining the expression level of two biomarkers and at least one endogenous control gene in RNA isolated from a biological sample from the subject, wherein the two biomarkers comprise B2M and NAMPT; b) normalizing the expression of the biomarkers to the expression level of the at least one endogenous control gene; and c) determining that the subject is undergoing kidney transplant rejection based on the normalized expression levels from step (b).

The present disclosure provides a method of determining that a subject who has undergone kidney transplant is undergoing a transplant rejection, the method comprising: a) determining the expression level of two biomarkers in RNA isolated from a biological sample from the subject, wherein the two biomarkers comprise B2M and NAMPT; b) inputting the expression levels from step (a) into an algorithm to generate a score; and c) determining that the subject is undergoing kidney transplant rejection based on the score.

The present disclosure provides a method of determining that a subject who has undergone kidney transplant is undergoing a transplant rejection, the method comprising: a) determining the expression level of two biomarkers in RNA isolated from a biological sample from the subject, wherein the two biomarkers B2M and NAMPT; and b) determining that the subject is undergoing kidney transplant rejection based on the expression levels from step (a).

In some aspects, the RNA isolated from a biological sample from the subject can comprise cell-free RNA. In some aspects, the RNA isolated from a biological sample from the subject can comprise microvesicular RNA.

Accordingly, the present disclosure provides a method of determining the risk of a kidney transplant rejection in a subject who has undergone a kidney transplant, the method comprising: a) determining the expression level of two biomarkers and at least one endogenous control gene in microvesicular RNA isolated from a biological sample from the subject, wherein the two biomarkers comprise B2M and NAMPT; b) normalizing the expression of the biomarkers to the expression level of the at least one endogenous control gene; c) inputting the normalized expression levels from step (b) into an algorithm to generate a score; and d) determining the risk of a kidney transplant rejection in the subject based on the score.

The present disclosure provides a method of determining the risk of a kidney transplant rejection in a subject who has undergone a kidney transplant, the method comprising: a) determining the expression level of two biomarkers and at least one endogenous control gene in microvesicular RNA isolated from a biological sample from the subject, wherein the two biomarkers comprise B2M and NAMPT; b) normalizing the expression of the biomarkers to the expression level of the at least one endogenous control gene; and c) determining the risk of a kidney transplant rejection in the subject based on the normalized expression levels from step (b).

The present disclosure provides a method of determining the risk of a kidney transplant rejection in a subject who has undergone a kidney transplant, the method comprising: a) determining the expression level of two biomarkers in microvesicular RNA isolated from a biological sample from the subject, wherein the two biomarkers comprise B2M and NAMPT; b) inputting the expression levels from step (a) into an algorithm to generate a score; and c) determining the risk of a kidney transplant rejection in the subject based on the score.

The present disclosure provides a method of determining the risk of a kidney transplant rejection in a subject who has undergone a kidney transplant, the method comprising: a) determining the expression level of two biomarkers in microvesicular RNA isolated from a biological sample from the subject, wherein the two biomarkers comprise B2M and NAMPT; and b) determining the risk of a kidney transplant rejection in the subject based on the expression levels from step (a).

In some aspects, the present disclosure provides a method of determining that a subject who has undergone kidney transplant is undergoing a transplant rejection, the method comprising: a) determining the expression level of two biomarkers and at least one endogenous control gene in microvesicular RNA isolated from a biological sample from the subject, wherein the two biomarkers comprise B2M and NAMPT; b) normalizing the expression of the biomarkers to the expression level of the at least one endogenous control gene; c) inputting the normalized expression levels from step (b) into an algorithm to generate a score; and d) determining that the subject is undergoing kidney transplant rejection based on the score.

The present disclosure provides a method of determining that a subject who has undergone kidney transplant is undergoing a transplant rejection, the method comprising: a) determining the expression level of two biomarkers and at least one endogenous control gene in microvesicular RNA isolated from a biological sample from the subject, wherein the two biomarkers comprise B2M and NAMPT; b) normalizing the expression of the biomarkers to the expression level of the at least one endogenous control gene; and c) determining that the subject is undergoing kidney transplant rejection based on the normalized expression levels from step (b).

The present disclosure provides a method of determining that a subject who has undergone kidney transplant is undergoing a transplant rejection, the method comprising: a) determining the expression level of two biomarkers in microvesicular RNA isolated from a biological sample from the subject, wherein the two biomarkers comprise B2M and NAMPT; b) inputting the expression levels from step (a) into an algorithm to generate a score; and c) determining that the subject is undergoing kidney transplant rejection based on the score.

The present disclosure provides a method of determining that a subject who has undergone kidney transplant is undergoing a transplant rejection, the method comprising: a) determining the expression level of two biomarkers in microvesicular RNA isolated from a biological sample from the subject, wherein the two biomarkers comprise B2M and NAMPT; and b) determining that the subject is undergoing kidney transplant rejection based on the expression levels from step (a).

In some aspects of the preceding methods, step (a) can further comprise determining the expression level of the biomarkers in cell-free DNA in addition to microvesicular RNA, and the subsequent steps of the method can incorporate the analysis of said cell-free DNA. Thus, in a non-limiting example, the present disclosure provides a method of determining the risk of a kidney transplant rejection in a subject who has undergone a kidney transplant, the method comprising: a) determining the expression level of two biomarkers and at least one endogenous control gene in microvesicular RNA and cell-free DNA isolated from a biological sample from the subject, wherein the two biomarkers comprise B2M and NAMPT; b) normalizing the expression of the biomarkers to the expression level of the at least one endogenous control gene; c) inputting the normalized expression levels from step (b) into an algorithm to generate a score; and d) determining the risk of a kidney transplant rejection in the subject based on the score.

In some aspects of the preceding methods, the subject is a subject that has no clinical indications of a kidney transplant rejection.

In some aspects of the preceding methods, determining the risk of a kidney transplant rejection in the subject based on the score can comprise: i) comparing the score to a predetermined cutoff value; and ii) determining that the at the subject is at a high risk of having a kidney transplant rejection when the score is greater than or equal to the predetermined cutoff value or determining that the subject is at low risk of having a kidney transplant rejection when the score is less than the predetermined cutoff value.

In some aspects, determining the risk of a kidney transplant rejection in a subject based on expression levels, normalized expressions levels or a score can comprise comparing the expression levels, normalized expression levels or a score to corresponding predetermined cutoff values and determining the risk of kidney transplant rejection based on the relationship between the expression levels, the normalized expression levels, and the score and the corresponding predetermined cutoff values (e.g. greater than, greater than or equal to, less than, less than or equal to, or equal to).

In some aspects, determining that the subject is undergoing kidney transplant rejection based on expression levels, normalized expressions levels or a score can comprise comparing the expression levels, normalized expression levels or a score to corresponding predetermined cutoff values and determining that the subject is undergoing kidney transplant rejection based on the relationship between the expression levels, the normalized expression levels, or the score and the corresponding predetermined cutoff values (e.g. greater than, greater than or equal to, less than, less than or equal to, or equal to).

In some aspects of the preceding methods, the algorithm can be the product of; a trained classifier built from at least one predictive classification algorithm, wherein the at least one predictive classification algorithm comprises naïve Bayes.

In some aspects, the trained classifier can comprise two univariate features. In some aspects, the two bivariate features are B2M and NAMPT.

In some aspects of the preceding methods, the trained classifier can be trained using the expression levels of the biomarkers measured in RNA (e.g. microvesicular RNA, cell-free RNA, etc.) isolated from a training set of biological samples. In some aspects, a training set of biological samples can comprise: i) a first plurality of biological samples isolated from subjects that have no clinical indications of a kidney transplant rejection.

In some aspects of the preceding methods, a portion of the biological samples in the first plurality of biological samples in the training set can be from subjects who are identified by biopsy to be positive for kidney transplant rejection and a portion of the biological samples in the first plurality of biological samples in the training set can be from subjects who are identified by biopsy to be negative for kidney transplant rejection.

In some aspects of the preceding methods, a training set of biological samples can further comprise a second plurality of biological samples isolated from subjects identified to be at risk for a kidney transplant rejection based on at least one clinical indications of kidney transplant rejection. In some aspects, this second plurality of biological samples can be from subjects identified by biopsy to be positive for kidney transplant rejection.

In some aspects of the preceding methods, an algorithm and a corresponding predetermined cutoff value can have, or be selected to have, a specificity for identifying kidney transplant rejection of at least about 90%. In some aspects of the preceding methods, an algorithm and a corresponding predetermined cutoff value can have, or be selected to have, a specificity for identifying kidney transplant rejection of at least about 95%.

### Classifier #6

The present disclosure provides methods of identifying the presence of TCMR or ABMR in a subject that has undergone a kidney transplant, the method comprising: a) determining the expression level of at least two of five biomarkers in microvesicular RNA isolated from a biological sample from the subject, wherein the five biomarkers comprise IL18BP, CXCL11, CD74, CD44 and C3; and b) identifying the presence of TCMR or the presence of ABMR in the subject based on the expression levels measured in step (a). In some aspects, identifying the presence of TCMR or the presence of ABMR in a subject based on the expression levels measured in step (a) can comprise comparing the one or more expression levels to corresponding predetermined cutoff values and determining the presence of TCMR or the presence of ABMR based on the relationship between the one or more expression levels and the corresponding predetermined cutoff values (e.g. greater than, greater than or equal to, less than, less than or equal to, or equal to).

The present disclosure provides methods of identifying the absence of ABMR in a subject that has undergone a kidney transplant, the method comprising: a) determining the expression level of at least two of five biomarkers in microvesicular RNA isolated from a biological sample from the subject, wherein the five biomarkers comprise IL18BP, CXCL11, CD74, CD44 and C3; and b) identifying the absence of ABMR in the subject based on the expression levels measured in step (a). In some aspects, identifying the absence of ABMR in a subject based on the expression levels measured in step (a) can comprise comparing the one or more expression levels to corresponding predetermined cutoff values and determining the absence of ABMR based on the relationship between the one or more expression levels and the corresponding predetermined cutoff values (e.g. greater than, greater than or equal to, less than, less than or equal to, or equal to).

The present disclosure provides methods of identifying a subject that has undergone a kidney transplant is at low risk of having ABMR, the method comprising: a) determining the expression level of at least two of five biomarkers in microvesicular RNA isolated from a biological sample from the subject, wherein the five biomarkers comprise IL18BP, CXCL11, CD74, CD44 and C3; and b) identifying that the subject is at low risk of having ABMR based on the expression levels measured in step (a). In some aspects, identifying that the subject is at low risk of having ABMR based on the expression levels measured in step (a) can comprise comparing the one or more expression levels to corresponding predetermined cutoff values and determining that the subject is at low risk of having ABMR based on the relationship between the one or more expression levels and the corresponding predetermined cutoff values (e.g. greater than, greater than or equal to, less than, less than or equal to, or equal to).

The present disclosure provides methods of identifying a subject that has undergone a kidney transplant is at high risk of having ABMR, the method comprising: a) determining the expression level of at least two of five biomarkers in microvesicular RNA isolated from a biological sample from the subject, wherein the five biomarkers comprise IL18BP, CXCL11, CD74, CD44 and C3; and b) identifying that the subject is at high risk of having ABMR based on the expression levels measured in step (a). In some aspects, identifying that the subject is at high risk of having ABMR based on the expression levels measured in step (a) can comprise comparing the one or more expression levels to corresponding predetermined cutoff values and determining that the subject is at high risk of having ABMR based on the relationship between the one or more expression levels and the corresponding predetermined cutoff values (e.g. greater than, greater than or equal to, less than, less than or equal to, or equal to).

The present disclosure provides methods of identifying the risk of ABMR in a subject that has undergone a kidney transplant, the method comprising: a) determining the expression level of at least two of five biomarkers in microvesicular RNA isolated from a biological sample from the subject, wherein the five biomarkers comprise IL18BP, CXCL11, CD74, CD44 and C3; and b) identifying the risk of ABMR in the subject based on the expression levels measured in step (a). In some aspects, identifying the risk of ABMR in the subject based on the expression levels measured in step (a) can comprise comparing the one or more expression levels to corresponding predetermined cutoff values and determining the risk based on the relationship between the one or more expression levels and the corresponding predetermined cutoff values (e.g. greater than, greater than or equal to, less than, less than or equal to, or equal to).

The present disclosure provides methods of identifying the presence of TCMR or ABMR in a subject that has undergone a kidney transplant, the method comprising: a) determining the expression level of at least two of five biomarkers in microvesicular RNA isolated from a biological sample from the subject, wherein the five biomarkers comprise IL18BP, CXCL11, CD74, CD44 and C3; b) inputting the expression levels from step (a) into an algorithm to generate a score; c) identifying the presence of TCMR or the presence of ABMR in the subject based on the score. In some aspects, identifying the presence of TCMR or the presence of ABMR in the subject based on a score can comprise comparing the score to a predetermined cutoff value; and determining the presence of TCMR or the presence of ABMR based on the relationship between the score and the predetermined cutoff value (e.g. is the score greater than, greater than or equal to, less than, or less than or equal to the predetermined cutoff value).

Accordingly, the present disclosure provides methods of identifying the presence of TCMR or ABMR in a subject that has undergone a kidney transplant, the method comprising: a) determining the expression level of at least two of five biomarkers in microvesicular RNA isolated from a biological sample from the subject, wherein the five biomarkers comprise IL18BP, CXCL11, CD74, CD44 and C3; b) inputting the expression levels from step (a) into an algorithm to generate a score; c) comparing the score to a predetermined cutoff value; and d) identifying the presence of TCMR or the presence of ABMR in the subject based on the relationship between the score and the predetermined cutoff value.

Accordingly, the present disclosure provides methods of identifying the presence of TCMR or ABMR in a subject that has undergone a kidney transplant, the method comprising: a) determining the expression level of at least two of five biomarkers in microvesicular RNA isolated from a biological sample from the subject, wherein the five biomarkers comprise IL18BP, CXCL11, CD74, CD44 and C3; b) inputting the expression levels from step (a) into an algorithm to generate a score; c) comparing the score to a predetermined cutoff value; and d) identifying the presence of TCMR when the score is less than or equal to the corresponding predetermined cutoff value or the presence of ABMR in the subject when the score is greater than the predetermined cutoff value.

Accordingly, the present disclosure provides methods of identifying the presence of TCMR or ABMR in a subject that has undergone a kidney transplant, the method comprising: a) determining the expression level of at least two of five biomarkers in microvesicular RNA isolated from a biological sample from the subject, wherein the five biomarkers comprise IL18BP, CXCL11, CD74, CD44 and C3; b) inputting the expression levels from step (a) into an algorithm to generate a score; c) comparing the score to a predetermined cutoff value; and d) identifying the presence of TCMR when the score is less than the corresponding predetermined cutoff value or the presence of ABMR in the subject when the score is greater than or equal to the predetermined cutoff value.

Accordingly, the present disclosure provides methods of identifying the presence of TCMR or ABMR in a subject that has undergone a kidney transplant, the method comprising: a) determining the expression level of at least two of five biomarkers in microvesicular RNA isolated from a biological sample from the subject, wherein the five biomarkers comprise IL18BP, CXCL11, CD74, CD44 and C3; b) inputting the expression levels from step (a) into an algorithm to generate a score; c) comparing the score to a predetermined cutoff value; and d) identifying the presence of ABMR when the score is less than or equal to the corresponding predetermined cutoff value or the presence of TCMR in the subject when the score is greater than the predetermined cutoff value.

Accordingly, the present disclosure provides methods of identifying the presence of TCMR or ABMR in a subject that has undergone a kidney transplant, the method comprising: a) determining the expression level of at least two of five biomarkers in microvesicular RNA isolated from a biological sample from the subject, wherein the five biomarkers comprise IL18BP, CXCL11, CD74, CD44 and C3; b) inputting the expression levels from step (a) into an algorithm to generate a score; c) comparing the score to a predetermined cutoff value; and d) identifying the presence of ABMR when the score is less than the corresponding predetermined cutoff value or the presence of TCMR in the subject when the score is greater than or equal to the predetermined cutoff value.

The present disclosure provides methods of identifying the absence of ABMR in a subject that has undergone a kidney transplant, the method comprising: a) determining the expression level of at least two of five biomarkers in microvesicular RNA isolated from a biological sample from the subject, wherein the five biomarkers comprise IL18BP, CXCL11, CD74, CD44 and C3; b) inputting the expression levels from step (a) into an algorithm to generate a score; c) identifying the absence of ABMR in the subject based on the score. In some aspects, identifying the absence of ABMR in the subject based on a score can comprise comparing the score to a predetermined cutoff value; and determining the absence of ABMR based on the relationship between the score and the predetermined cutoff value (e.g. is the score greater than, greater than or equal to, less than, or less than or equal to the predetermined cutoff value).

Accordingly, the present disclosure provides methods of identifying the absence of ABMR in a subject that has undergone a kidney transplant, the method comprising: a) determining the expression level of at least two of five biomarkers in microvesicular RNA isolated from a biological sample from the subject, wherein the five biomarkers comprise IL18BP, CXCL11, CD74, CD44 and C3; b) inputting the expression levels from step (a) into an algorithm to generate a score; c) comparing the score to a predetermined cutoff value; and d) identifying the absence of ABMR when the score is less than the corresponding predetermined cutoff value.

Accordingly, the present disclosure provides methods of identifying the absence of ABMR in a subject that has undergone a kidney transplant, the method comprising: a) determining the expression level of at least two of five biomarkers in microvesicular RNA isolated from a biological sample from the subject, wherein the five biomarkers comprise IL18BP, CXCL11, CD74, CD44 and C3; b) inputting the expression levels from step (a) into an algorithm to generate a score; c) comparing the score to a predetermined cutoff value; and d) identifying the absence of ABMR when the score is less than or equal to the corresponding predetermined cutoff value.

The present disclosure provides methods of identifying a subject that has undergone a kidney transplant is at low risk of having ABMR, the method comprising: a) determining the expression level of at least two of five biomarkers in microvesicular RNA isolated from a biological sample from the subject, wherein the five biomarkers comprise IL18BP, CXCL11, CD74, CD44 and C3; b) inputting the expression levels from step (a) into an algorithm to generate a score; c) identifying that the subject is at low risk of having ABMR based on the score. In some aspects, identifying that the subject is at low risk of having ABMR based on a score can comprise comparing the score to a predetermined cutoff value; and determining that the subject is at low risk of having ABMR based on the relationship between the score and the predetermined cutoff value (e.g. is the score greater than, greater than or equal to, less than, or less than or equal to the predetermined cutoff value).

Accordingly, the present disclosure provides methods of identifying a subject that has undergone a kidney transplant is at low risk of having ABMR, the method comprising: a) determining the expression level of at least two of five biomarkers in microvesicular RNA isolated from a biological sample from the subject, wherein the five biomarkers comprise IL18BP, CXCL11, CD74, CD44 and C3; b) inputting the expression levels from step (a) into an algorithm to generate a score; c) comparing the score to a predetermined cutoff value; and d) identifying that the subject is at low risk of having ABMR when the score is less than the corresponding predetermined cutoff value.

Accordingly, the present disclosure provides methods of identifying a subject that has undergone a kidney transplant is at low risk of having ABMR, the method comprising: a) determining the expression level of at least two of five biomarkers in microvesicular RNA isolated from a biological sample from the subject, wherein the five biomarkers comprise IL18BP, CXCL11, CD74, CD44 and C3; b) inputting the expression levels from step (a) into an algorithm to generate a score; c) comparing the score to a predetermined cutoff value; and d) identifying that the subject is at low risk of having ABMR when the score is less than or equal to the corresponding predetermined cutoff value.

The present disclosure provides methods of identifying a subject that has undergone a kidney transplant is at high risk of having ABMR, the method comprising: a) determining the expression level of at least two of five biomarkers in microvesicular RNA isolated from a biological sample from the subject, wherein the five biomarkers comprise IL18BP, CXCL11, CD74, CD44 and C3; b) inputting the expression levels from step (a) into an algorithm to generate a score; c) identifying that the subject is at high risk of having ABMR based on the score. In some aspects, identifying that the subject is at high risk of having ABMR based on a score can comprise comparing the score to a predetermined cutoff value; and determining that the subject is at high risk of having ABMR based on the relationship between the score and the predetermined cutoff value (e.g. is the score greater than, greater than or equal to, less than, or less than or equal to the predetermined cutoff value).

The present disclosure provides methods of identifying the risk of ABMR in a subject that has undergone a kidney transplant, the method comprising: a) determining the expression level of at least two of five biomarkers in microvesicular RNA isolated from a biological sample from the subject, wherein the five biomarkers comprise IL18BP, CXCL11, CD74, CD44 and C3; b) inputting the expression levels from step (a) into an algorithm to generate a score; c) identifying the risk ABMR in the subject based on the score. In some aspects, identifying the risk of ABMR in the subject based on a score can comprise comparing the score to a predetermined cutoff value; and determining the risk based on the relationship between the score and the predetermined cutoff value (e.g. is the score greater than, greater than or equal to, less than, or less than or equal to the predetermined cutoff value).

The present disclosure provides methods of identifying the presence of TCMR or ABMR in a subject that has undergone a kidney transplant, the method comprising: a) determining the expression level of at least two of five biomarkers in microvesicular RNA and cell-free DNA isolated from a biological sample from the subject, wherein the five biomarkers comprise IL18BP, CXCL11, CD74, CD44 and C3; and b) identifying the presence of TCMR or the presence of ABMR in the subject based on the expression levels measured in step (a). In some aspects, identifying the presence of TCMR or the presence of ABMR in a subject based on the expression levels measured in step (a) can comprise comparing the one or more expression levels to corresponding predetermined cutoff values and determining the presence of TCMR or the presence of ABMR based on the relationship between the one or more expression levels and the corresponding predetermined cutoff values (e.g. greater than, greater than or equal to, less than, less than or equal to, or equal to).

The present disclosure provides methods of identifying the absence of ABMR in a subject that has undergone a kidney transplant, the method comprising: a) determining the expression level of at least two of five biomarkers in microvesicular RNA and cell-free DNA isolated from a biological sample from the subject, wherein the five biomarkers comprise IL18BP, CXCL11, CD74, CD44 and C3; and b) identifying the absence of ABMR in the subject based on the expression levels measured in step (a). In some aspects, identifying the absence of ABMR in a subject based on the expression levels measured in step (a) can comprise comparing the one or more expression levels to corresponding predetermined cutoff values and determining the absence of ABMR based on the relationship between the one or more expression levels and the corresponding predetermined cutoff values (e.g. greater than, greater than or equal to, less than, less than or equal to, or equal to).

The present disclosure provides methods of identifying a subject that has undergone a kidney transplant is at low risk of having ABMR, the method comprising: a) determining the expression level of at least two of five biomarkers in microvesicular RNA and isolated from a biological sample from the subject, wherein the five biomarkers comprise IL18BP, CXCL11, CD74, CD44 and C3; and b) identifying that the subject is at low risk of having ABMR based on the expression levels measured in step (a). In some aspects, identifying that the subject is at low risk of having ABMR based on the expression levels measured in step (a) can comprise comparing the one or more expression levels to corresponding predetermined cutoff values and determining that the subject is at low risk of having ABMR based on the relationship between the one or more expression levels and the corresponding predetermined cutoff values (e.g. greater than, greater than or equal to, less than, less than or equal to, or equal to).

The present disclosure provides methods of identifying a subject that has undergone a kidney transplant is at high risk of having ABMR, the method comprising: a) determining the expression level of at least two of five biomarkers in microvesicular RNA and cell-free DNA isolated from a biological sample from the subject, wherein the five biomarkers comprise IL18BP, CXCL11, CD74, CD44 and C3; and b) identifying that the subject is at high risk of having ABMR based on the expression levels measured in step (a). In some aspects, identifying that the subject is at high risk of having ABMR based on the expression levels measured in step (a) can comprise comparing the one or more expression levels to corresponding predetermined cutoff values and determining that the subject is at high risk of having ABMR based on the relationship between the one or more expression levels and the corresponding predetermined cutoff values (e.g. greater than, greater than or equal to, less than, less than or equal to, or equal to).

The present disclosure provides methods of identifying the risk of ABMR in a subject that has undergone a kidney transplant, the method comprising: a) determining the expression level of at least two of five biomarkers in microvesicular RNA and cell-free DNA isolated from a biological sample from the subject, wherein the five biomarkers comprise IL18BP, CXCL11, CD74, CD44 and C3; and b) identifying the risk of ABMR in the subject based on the expression levels measured in step (a). In some aspects, identifying the risk of ABMR in the subject based on the expression levels measured in step (a) can comprise comparing the one or more expression levels to corresponding predetermined cutoff values and determining the risk based on the relationship between the one or more expression levels and the corresponding predetermined cutoff values (e.g. greater than, greater than or equal to, less than, less than or equal to, or equal to).

The present disclosure provides methods of identifying the presence of TCMR or ABMR in a subject that has undergone a kidney transplant, the method comprising: a) determining the expression level of at least two of five biomarkers in microvesicular RNA and cell-free DNA isolated from a biological sample from the subject, wherein the five biomarkers comprise IL18BP, CXCL11, CD74, CD44 and C3; b) inputting the expression levels from step (a) into an algorithm to generate a score; c) identifying the presence of TCMR or the presence of ABMR in the subject based on the score. In some aspects, identifying the presence of TCMR or the presence of ABMR in the subject based on a score can comprise comparing the score to a predetermined cutoff value; and determining the presence of TCMR or the presence of ABMR based on the relationship between the score and the predetermined cutoff value (e.g. is the score greater than, greater than or equal to, less than, or less than or equal to the predetermined cutoff value).

Accordingly, the present disclosure provides methods of identifying the presence of TCMR or ABMR in a subject that has undergone a kidney transplant, the method comprising: a) determining the expression level of at least two of five biomarkers in microvesicular RNA and cell-free DNA isolated from a biological sample from the subject, wherein the five biomarkers comprise IL18BP, CXCL11, CD74, CD44 and C3; b) inputting the expression levels from step (a) into an algorithm to generate a score; c) comparing the score to a predetermined cutoff value; and d) identifying the presence of TCMR or the presence of ABMR in the subject based on the relationship between the score and the predetermined cutoff value.

Accordingly, the present disclosure provides methods of identifying the presence of TCMR or ABMR in a subject that has undergone a kidney transplant, the method comprising: a) determining the expression level of at least two of five biomarkers in microvesicular RNA and cell-free DNA isolated from a biological sample from the subject, wherein the five biomarkers comprise IL18BP, CXCL11, CD74, CD44 and C3; b) inputting the expression levels from step (a) into an algorithm to generate a score; c) comparing the score to a predetermined cutoff value; and d) identifying the presence of TCMR when the score is less than or equal to the corresponding predetermined cutoff value or the presence of ABMR in the subject when the score is greater than the predetermined cutoff value.

Accordingly, the present disclosure provides methods of identifying the presence of TCMR or ABMR in a subject that has undergone a kidney transplant, the method comprising: a) determining the expression level of at least two of five biomarkers in microvesicular RNA and cell-free DNA isolated from a biological sample from the subject, wherein the five biomarkers comprise IL18BP, CXCL11, CD74, CD44 and C3; b) inputting the expression levels from step (a) into an algorithm to generate a score; c) comparing the score to a predetermined cutoff value; and d) identifying the presence of TCMR when the score is less than the corresponding predetermined cutoff value or the presence of ABMR in the subject when the score is greater than or equal to the predetermined cutoff value.

Accordingly, the present disclosure provides methods of identifying the presence of TCMR or ABMR in a subject that has undergone a kidney transplant, the method comprising: a) determining the expression level of at least two of five biomarkers in microvesicular RNA and cell-free DNA isolated from a biological sample from the subject, wherein the five biomarkers comprise IL18BP, CXCL11, CD74, CD44 and C3; b) inputting the expression levels from step (a) into an algorithm to generate a score; c) comparing the score to a predetermined cutoff value; and d) identifying the presence of ABMR when the score is less than or equal to the corresponding predetermined cutoff value or the presence of TCMR in the subject when the score is greater than the predetermined cutoff value.

Accordingly, the present disclosure provides methods of identifying the presence of TCMR or ABMR in a subject that has undergone a kidney transplant, the method comprising: a) determining the expression level of at least two of five biomarkers in microvesicular RNA and cell-free DNA isolated from a biological sample from the subject, wherein the five biomarkers comprise IL18BP, CXCL11, CD74, CD44 and C3; b) inputting the expression levels from step (a) into an algorithm to generate a score; c) comparing the score to a predetermined cutoff value; and d) identifying the presence of ABMR when the score is less than the corresponding predetermined cutoff value or the presence of TCMR in the subject when the score is greater than or equal to the predetermined cutoff value.

The present disclosure provides methods of identifying the absence of ABMR in a subject that has undergone a kidney transplant, the method comprising: a) determining the expression level of at least two of five biomarkers in microvesicular RNA and cell-free DNA isolated from a biological sample from the subject, wherein the five biomarkers comprise IL18BP, CXCL11, CD74, CD44 and C3; b) inputting the expression levels from step (a) into an algorithm to generate a score; c) identifying the absence of ABMR in the subject based on the score. In some aspects, identifying the absence of ABMR in the subject based on a score can comprise comparing the score to a predetermined cutoff value; and determining the absence of ABMR based on the relationship between the score and the predetermined cutoff value (e.g. is the score greater than, greater than or equal to, less than, or less than or equal to the predetermined cutoff value).

Accordingly, the present disclosure provides methods of identifying the absence of ABMR in a subject that has undergone a kidney transplant, the method comprising: a) determining the expression level of at least two of five biomarkers in microvesicular RNA and cell-free DNA isolated from a biological sample from the subject, wherein the five biomarkers comprise IL18BP, CXCL11, CD74, CD44 and C3; b) inputting the expression levels from step (a) into an algorithm to generate a score; c) comparing the score to a predetermined cutoff value; and d) identifying the absence of ABMR when the score is less than the corresponding predetermined cutoff value.

Accordingly, the present disclosure provides methods of identifying the absence of ABMR in a subject that has undergone a kidney transplant, the method comprising: a) determining the expression level of at least two of five biomarkers in microvesicular RNA and cell-free DNA isolated from a biological sample from the subject, wherein the five biomarkers comprise IL18BP, CXCL11, CD74, CD44 and C3; b) inputting the expression levels from step (a) into an algorithm to generate a score; c) comparing the score to a predetermined cutoff value; and d) identifying the absence of ABMR when the score is less than or equal to the corresponding predetermined cutoff value.

The present disclosure provides methods of identifying a subject that has undergone a kidney transplant is at low risk of having ABMR, the method comprising: a) determining the expression level of at least two of five biomarkers in microvesicular RNA and cell-free DNA isolated from a biological sample from the subject, wherein the five biomarkers comprise IL18BP, CXCL11, CD74, CD44 and C3; b) inputting the expression levels from step (a) into an algorithm to generate a score; c) identifying that the subject is at low risk of having ABMR based on the score. In some aspects, identifying that the subject is at low risk of having ABMR based on a score can comprise comparing the score to a predetermined cutoff value; and determining that the subject is at low risk of having ABMR based on the relationship between the score and the predetermined cutoff value (e.g. is the score greater than, greater than or equal to, less than, or less than or equal to the predetermined cutoff value).

Accordingly, the present disclosure provides methods of identifying a subject that has undergone a kidney transplant is at low risk of having ABMR, the method comprising: a) determining the expression level of at least two of five biomarkers in microvesicular RNA and cell-free DNA isolated from a biological sample from the subject, wherein the five biomarkers comprise IL18BP, CXCL11, CD74, CD44 and C3; b) inputting the expression levels from step (a) into an algorithm to generate a score; c) comparing the score to a predetermined cutoff value; and d) identifying that the subject is at low risk of having ABMR when the score is less than the corresponding predetermined cutoff value.

Accordingly, the present disclosure provides methods of identifying a subject that has undergone a kidney transplant is at low risk of having ABMR, the method comprising: a) determining the expression level of at least two of five biomarkers in microvesicular RNA and cell-free DNA isolated from a biological sample from the subject, wherein the five biomarkers comprise IL18BP, CXCL11, CD74, CD44 and C3; b) inputting the expression levels from step (a) into an algorithm to generate a score; c) comparing the score to a predetermined cutoff value; and d) identifying that the subject is at low risk of having ABMR when the score is less than or equal to the corresponding predetermined cutoff value.

The present disclosure provides methods of identifying a subject that has undergone a kidney transplant is at high risk of having ABMR, the method comprising: a) determining the expression level of at least two of five biomarkers in microvesicular RNA and cell-free DNA isolated from a biological sample from the subject, wherein the five biomarkers comprise IL18BP, CXCL11, CD74, CD44 and C3; b) inputting the expression levels from step (a) into an algorithm to generate a score; c) identifying that the subject is at high risk of having ABMR based on the score. In some aspects, identifying that the subject is at high risk of having ABMR based on a score can comprise comparing the score to a predetermined cutoff value; and determining that the subject is at high risk of having ABMR based on the relationship between the score and the predetermined cutoff value (e.g. is the score greater than, greater than or equal to, less than, or less than or equal to the predetermined cutoff value).

The present disclosure provides methods of identifying the risk of ABMR in a subject that has undergone a kidney transplant, the method comprising: a) determining the expression level of at least two of five biomarkers in microvesicular RNA and cell-free DNA isolated from a biological sample from the subject, wherein the five biomarkers comprise IL18BP, CXCL11, CD74, CD44 and C3; b) inputting the expression levels from step (a) into an algorithm to generate a score; c) identifying the risk of ABMR in the subject based on the score. In some aspects, identifying the risk of ABMR in the subject based on a score can comprise comparing the score to a predetermined cutoff value; and determining the risk based on the relationship between the score and the predetermined cutoff value (e.g. is the score greater than, greater than or equal to, less than, or less than or equal to the predetermined cutoff value).

In some aspects of the preceding methods, step (a) can comprise determining the expression level of at least three, or at least four, or each of the 5 biomarkers.

In some aspects of the preceding methods, step (a) can comprise determining the expression level of:
(i) IL18BP and CXCL11;
(ii) IL18BP and CD74;
(iii) IL18BP and CD44;
(iv) IL18BP and C3;
(v) CXCL11 and CD74;
(vi) CXCL11 and CD44;
(vii) CXCL11 and C3;
(viii) CD74 and CD44;
(ix) CD74 and C3; or
(x) CD44 and C3.

In some aspects of the preceding methods, step (a) can comprise determining the expression level of:
(i) IL18BP, CXCL11 and CD74;
(ii) IL18BP, CXCL11 and CD44;
(iii) IL18BP, CXCL11 and C3;
(iv) IL18BP, CD74 and CD44;
(v) IL18BP, CD74 and C3;
(vi) IL18BP, CD44 and C3;
(vii) CXCL11, CD74 and CD44;
(viii) CXCL11, CD74 and C3;
(ix) CXCL11, CD44 and C3; or
(x) CD74, CD44 and C3.

In some aspects of the preceding methods, step (a) can comprise determining the expression level of:
(i) IL18BP, CXCL11, CD74 and CD44;
(ii) IL18BP, CXCL11, CD74 and C3;
(iii) IL18BP, CXCL11, CD44 and C3;
(iv) IL18BP, CD74, CD44 and C3; or
(v) CXCL11, CD74, CD44 and C3.

The present disclosure provides methods of identifying the presence of TCMR or ABMR in a subject that has undergone a kidney transplant, the method comprising: a) determining the expression level of at least one of five biomarkers in microvesicular RNA isolated from a biological sample from the subject, wherein the five biomarkers comprise IL18BP, CXCL11, CD74, CD44 and C3; and b) identifying the presence of TCMR or the presence of ABMR in the subject based on the expression levels measured in step (a). In some aspects, identifying the presence of TCMR or the presence of ABMR in a subject based on the expression levels measured in step (a) can comprise comparing the one or more expression levels to corresponding predetermined cutoff values and determining the presence of TCMR or the presence of ABMR based on the relationship between the one or more expression levels and the corresponding predetermined cutoff values (e.g. greater than, greater than or equal to, less than, less than or equal to, or equal to).

In a non-limiting example, the present disclosure provides methods of identifying the presence of TCMR or ABMR in a subject that has undergone a kidney transplant, the method comprising: a) determining the expression level of IL18BP in microvesicular RNA isolated from a biological sample from the subject; b) comparing the expression level of IL18BP to a predetermined cutoff value; and c) identifying the presence of TCMR or the presence of ABMR in the subject based on the relationship between the expression level of IL18BP and the predetermined cutoff value.

In a non-limiting example, the present disclosure provides methods of identifying the presence of TCMR or ABMR in a subject that has undergone a kidney transplant, the method comprising: a) determining the expression level of IL18BP in microvesicular RNA isolated from a biological sample from the subject; b) comparing the expression level of IL18BP to a predetermined cutoff value; and c) identifying the presence of TCMR in the subject when the expression level of IL18BP is greater than or equal to the predetermined cutoff value or the presence of ABMR in the subject when the expression level of IL18BP is less than the predetermined cutoff value.

The present disclosure provides methods of identifying the presence of TCMR or ABMR in a subject that has undergone a kidney transplant, the method comprising: a) determining the expression level of at least one of five biomarkers in microvesicular RNA isolated from a biological sample from the subject, wherein the five biomarkers comprise IL18BP. CXCL11, CD74, CD44 and C3; b) inputting the expression levels from step (a) into an algorithm to generate a score; c) identifying the presence of TCMR or the presence of ABMR in the subject based on the score. In some aspects, identifying the presence of TCMR or the presence of ABMR in the subject based on a score can comprise comparing the score to a predetermined cutoff value; and determining the presence of TCMR or the presence of ABMR based on the relationship between the score and the predetermined cutoff value (e.g. is the score greater than, greater than or equal to, less than, or less than or equal to the predetermined cutoff value).

In a non-limiting example, the present disclosure provides methods of identifying the presence of TCMR or ABMR in a subject that has undergone a kidney transplant, the method comprising: a) determining the expression level of IL18BP in microvesicular RNA isolated from a biological sample from the subject; b) inputting the expression level of IL18BP into an algorithm to generate a score; c) identifying the presence of TCMR or the presence of ABMR in the subject based on the score.

The present disclosure provides methods of identifying the absence of ABMR in a subject that has undergone a kidney transplant, the method comprising: a) determining the expression level of at least one of five biomarkers in microvesicular RNA isolated from a biological sample from the subject, wherein the five biomarkers comprise IL18BP, CXCL11, CD74, CD44 and C3; and b) identifying the absence of ABMR in the subject based on the expression levels measured in step (a). In some aspects, identifying absence of ABMR in a subject based on the expression levels measured in step (a) can comprise comparing the one or more expression levels to corresponding predetermined cutoff values and determining the absence of ABMR based on the relationship between the one or more expression levels and the corresponding predetermined cutoff values (e.g. greater than, greater than or equal to, less than, less than or equal to, or equal to).

In a non-limiting example, the present disclosure provides methods of identifying the absence of ABMR in a subject that has undergone a kidney transplant, the method comprising: a) determining the expression level of IL18BP in microvesicular RNA isolated from a biological sample from the subject; b) comparing the expression level of IL18BP to a predetermined cutoff value; and c) identifying absence of ABMR in the subject based on the relationship between the expression level of IL18BP and the predetermined cutoff value.

In a non-limiting example, the present disclosure provides methods of identifying the absence of ABMR in a subject that has undergone a kidney transplant, the method comprising: a) determining the expression level of IL18BP in microvesicular RNA isolated from a biological sample from the subject; b) comparing the expression level of IL18BP to a predetermined cutoff value; and c) identifying absence of ABMR in the subject when the expression level of IL18BP is greater than the predetermined cutoff value.

The present disclosure provides methods of identifying the absence of ABMR in a subject that has undergone a kidney transplant, the method comprising: a) determining the expression level of at least one of five biomarkers in microvesicular RNA isolated from a biological sample from the subject, wherein the five biomarkers comprise IL18BP, CXCL11, CD74, CD44 and C3; b) inputting the expression levels from step (a) into an algorithm to generate a score; c) identifying absence of ABMR in the subject based on the score. In some aspects, identifying absence of ABMR in the subject based on a score can comprise comparing the score to a predetermined cutoff value; and determining absence of ABMR based on the relationship between the score and the predetermined cutoff value (e.g. is the score greater than, greater than or equal to, less than, or less than or equal to the predetermined cutoff value).

In a non-limiting example, the present disclosure provides methods of identifying the absence of ABMR in a subject that has undergone a kidney transplant, the method comprising: a) determining the expression level of IL18BP in microvesicular RNA isolated from a biological sample from the subject; b) inputting the expression level of IL18BP into an algorithm to generate a score; c) identifying absence of ABMR in the subject based on the score.

The present disclosure provides methods of identifying that a subject that has undergone a kidney transplant is at low risk of having ABMR, the method comprising: a) determining the expression level of at least one of five biomarkers in microvesicular RNA isolated from a biological sample from the subject, wherein the five biomarkers comprise IL18BP, CXCL11, CD74, CD44 and C3; and b) identifying that the subject is at low risk of having ABMR based on the expression levels measured in step (a). In some aspects, identifying that the subject is at low risk of having ABMR based on the expression levels measured in step (a) can comprise comparing the one or more expression levels to corresponding predetermined cutoff values and determining that the subject is at low risk of having ABMR based on the relationship between the one or more expression levels and the corresponding predetermined cutoff values (e.g. greater than, greater than or equal to, less than, less than or equal to, or equal to).

The present disclosure provides methods of identifying that a subject that has undergone a kidney transplant is at high risk of having ABMR, the method comprising: a) determining the expression level of at least one of five biomarkers in microvesicular RNA isolated from a biological sample from the subject, wherein the five biomarkers comprise IL18BP, CXCL11, CD74, CD44 and C3; and b) identifying that the subject is at high risk of having ABMR based on the expression levels measured in step (a). In some aspects, identifying that the subject is at high risk of having ABMR based on the expression levels measured in step (a) can comprise comparing the one or more expression levels to corresponding predetermined cutoff values and determining that the subject is at high risk of having ABMR based on the relationship between the one or more expression levels and the corresponding predetermined cutoff values (e.g. greater than, greater than or equal to, less than, less than or equal to, or equal to).

The present disclosure provides methods of identifying the risk of ABMR in a subject that has undergone a kidney transplant, the method comprising: a) determining the expression level of at least one of five biomarkers in microvesicular RNA isolated from a biological sample from the subject, wherein the five biomarkers comprise IL18BP, CXCL11, CD74, CD44 and C3; and b) identifying the risk of ABMR in the subject based on the expression levels measured in step (a). In some aspects, identifying the risk of ABMR in the subject based on the expression levels measured in step (a) can comprise comparing the one or more expression levels to corresponding predetermined cutoff values and determining the risk based on the relationship between the one or more expression levels and the corresponding predetermined cutoff values (e.g. greater than, greater than or equal to, less than, less than or equal to, or equal to).

In a non-limiting example, the present disclosure provides methods of identifying that a subject that has undergone a kidney transplant is at low risk of having ABMR, the method comprising: a) determining the expression level of IL18BP in microvesicular RNA isolated from a biological sample from the subject; b) comparing the expression level of IL18BP to a predetermined cutoff value; and c) identifying that the subject is at low risk of having ABMR based on the relationship between the expression level of IL18BP and the predetermined cutoff value.

In a non-limiting example, the present disclosure provides methods of identifying that a subject that has undergone a kidney transplant is at low risk of having ABMR, the method comprising: a) determining the expression level of IL18BP in microvesicular RNA isolated from a biological sample from the subject; b) comparing the expression level of IL18BP to a predetermined cutoff value; and c) identifying that the subject is at low risk of having ABMR when the expression level of IL18BP is greater than the predetermined cutoff value.

In a non-limiting example, the present disclosure provides methods of identifying that a subject that has undergone a kidney transplant is at high risk of having ABMR, the method comprising: a) determining the expression level of IL18BP in microvesicular RNA isolated from a biological sample from the subject; b) comparing the expression level of IL18BP to a predetermined cutoff value; and c) identifying that the subject is at high risk of having ABMR based on the relationship between the expression level of IL18BP and the predetermined cutoff value.

In a non-limiting example, the present disclosure provides methods of identifying the risk of ABMR in a subject that has undergone a kidney transplant, the method comprising: a) determining the expression level of IL18BP in microvesicular RNA isolated from a biological sample from the subject; b) comparing the expression level of IL18BP to a predetermined cutoff value; and c) identifying the risk of ABMR based on the relationship between the expression level of IL18BP and the predetermined cutoff value.

The present disclosure provides methods of identifying that a subject that has undergone a kidney transplant is at low risk of having ABMR, the method comprising: a) determining the expression level of at least one of five biomarkers in microvesicular RNA isolated from a biological sample from the subject, wherein the five biomarkers comprise IL18BP, CXCL11, CD74, CD44 and C3; b) inputting the expression levels from step (a) into an algorithm to generate a score; c) determining that the subject is at low risk of having ABMR based on the score. In some aspects, identifying that the subject is at low risk of having ABMR based on a score can comprise comparing the score to a predetermined cutoff value; and determining that the subject is at low risk of having ABMR based on the relationship between the score and the predetermined cutoff value (e.g. is the score greater than, greater than or equal to, less than, or less than or equal to the predetermined cutoff value).

The present disclosure provides methods of identifying that a subject that has undergone a kidney transplant is at high risk of having ABMR, the method comprising: a) determining the expression level of at least one of five biomarkers in microvesicular RNA isolated from a biological sample from the subject, wherein the five biomarkers comprise IL18BP, CXCL11, CD74, CD44 and C3; b) inputting the expression levels from step (a) into an algorithm to generate a score; c) determining that the subject is at high risk of having ABMR based on the score. In some aspects, identifying that the subject is at high risk of having ABMR based on a score can comprise comparing the score to a predetermined cutoff value; and determining that the subject is at high risk of having ABMR based on the relationship between the score and the predetermined cutoff value (e.g. is the score greater than, greater than or equal to, less than, or less than or equal to the predetermined cutoff value).

The present disclosure provides methods of identifying the risk of ABMR a subject that has undergone a kidney transplant, the method comprising: a) determining the expression level of at least one of five biomarkers in microvesicular RNA isolated from a biological sample from the subject, wherein the five biomarkers comprise IL18BP, CXCL11, CD74, CD44 and C3; b) inputting the expression levels from step (a) into an algorithm to generate a score; c) determining the risk of ABMR in the subject based on the score. In some aspects, identifying the risk of ABMR based on a score can comprise comparing the score to a predetermined cutoff value; and determining the risk based on the relationship between the score and the predetermined cutoff value (e.g. is the score greater than, greater than or equal to, less than, or less than or equal to the predetermined cutoff value).

In a non-limiting example, the present disclosure provides methods of identifying that a subject that has undergone a kidney transplant is at low risk of having ABMR, the method comprising: a) determining the expression level of IL18BP in microvesicular RNA isolated from a biological sample from the subject; b) inputting the expression level of IL18BP into an algorithm to generate a score; c) determining that the subject is at low risk of having ABMR based on the score.

In a non-limiting example, the present disclosure provides methods of identifying that a subject that has undergone a kidney transplant is at high risk of having ABMR, the method comprising: a) determining the expression level of IL18BP in microvesicular RNA isolated from a biological sample from the subject; b) inputting the expression level of IL18BP into an algorithm to generate a score; c) determining that the subject is at high risk of having ABMR based on the score.

In a non-limiting example, the present disclosure provides methods of identifying the risk of ABMR a subject that has undergone a kidney transplant, the method comprising: a) determining the expression level of IL18BP in microvesicular RNA isolated from a biological sample from the subject; b) inputting the expression level of IL18BP into an algorithm to generate a score; c) determining the risk of ABMR in the subject based on the score.

The present disclosure provides methods of identifying the presence of TCMR or ABMR in a subject that has undergone a kidney transplant, the method comprising: a) determining the expression level of at least one of five biomarkers in microvesicular RNA and cell-free DNA isolated from a biological sample from the subject, wherein the five biomarkers comprise IL18BP, CXCL11, CD74, CD44 and C3; and b) identifying the presence of TCMR or the presence of ABMR in the subject based on the expression levels measured in step (a). In some aspects, identifying the presence of TCMR or the presence of ABMR in a subject based on the expression levels measured in step (a) can comprise comparing the one or more expression levels to corresponding predetermined cutoff values and determining the presence of TCMR or the presence of ABMR based on the relationship between the one or more expression levels and the corresponding predetermined cutoff values (e.g. greater than, greater than or equal to, less than, less than or equal to, or equal to).

In a non-limiting example, the present disclosure provides methods of identifying the presence of TCMR or ABMR in a subject that has undergone a kidney transplant, the method comprising: a) determining the expression level of IL18BP in microvesicular RNA and cell-free DNA isolated from a biological sample from the subject; b) comparing the expression level of IL18BP to a predetermined cutoff value; and c) identifying the presence of TCMR or the presence of ABMR in the subject based on the relationship between the expression level of IL18BP and the predetermined cutoff value.

In a non-limiting example, the present disclosure provides methods of identifying the presence of TCMR or ABMR in a subject that has undergone a kidney transplant, the method comprising: a) determining the expression level of IL18BP in microvesicular RNA and cell-free DNA isolated from a biological sample from the subject; b) comparing the expression level of IL18BP to a predetermined cutoff value; and c) identifying the presence of TCMR in the subject when the expression level of IL18BP is greater than or equal to the predetermined cutoff value or identifying the presence of ABMR in the subject when the expression level of IL18BP is less than the predetermined cutoff value.

The present disclosure provides methods of identifying the presence of TCMR or ABMR in a subject that has undergone a kidney transplant, the method comprising: a) determining the expression level of at least one of five biomarkers in microvesicular RNA and cell-free DNA isolated from a biological sample from the subject, wherein the five biomarkers comprise IL18BP, CXCL11, CD74, CD44 and C3; b) inputting the expression levels from step (a) into an algorithm to generate a score; c) identifying the presence of TCMR or the presence of ABMR in the subject based on the score. In some aspects, identifying the presence of TCMR or the presence of ABMR in the subject based on a score can comprise comparing the score to a predetermined cutoff value; and determining the presence of TCMR or the presence of ABMR based on the relationship between the score and the predetermined cutoff value (e.g. is the score greater than, greater than or equal to, less than, or less than or equal to the predetermined cutoff value).

In a non-limiting example, the present disclosure provides methods of identifying the presence of TCMR or ABMR in a subject that has undergone a kidney transplant, the method comprising: a) determining the expression level of IL18BP in microvesicular RNA and cell-free DNA isolated from a biological sample from the subject; b) inputting the expression level of IL18BP into an algorithm to generate a score; c) identifying the presence of TCMR or the presence of ABMR in the subject based on the score.

The present disclosure provides methods of identifying the absence of ABMR in a subject that has undergone a kidney transplant, the method comprising: a) determining the expression level of at least one of five biomarkers in microvesicular RNA and cell-free DNA isolated from a biological sample from the subject, wherein the five biomarkers comprise IL18BP. CXCL11, CD74, CD44 and C3; and b) identifying the absence of ABMR in the subject based on the expression levels measured in step (a). In some aspects, identifying absence of ABMR in a subject based on the expression levels measured in step (a) can comprise comparing the one or more expression levels to corresponding predetermined cutoff values and determining the absence of ABMR based on the relationship between the one or more expression levels and the corresponding predetermined cutoff values (e.g. greater than, greater than or equal to, less than, less than or equal to, or equal to).

In a non-limiting example, the present disclosure provides methods of identifying the absence of ABMR in a subject that has undergone a kidney transplant, the method comprising: a) determining the expression level of IL18BP in microvesicular RNA and cell-free DNA isolated from a biological sample from the subject; b) comparing the expression level of IL18BP to a predetermined cutoff value; and c) identifying absence of ABMR in the subject based on the relationship between the expression level of IL18BP and the predetermined cutoff value.

In a non-limiting example, the present disclosure provides methods of identifying the absence of ABMR in a subject that has undergone a kidney transplant, the method comprising: a) determining the expression level of IL18BP in microvesicular RNA and cell-free DNA isolated from a biological sample from the subject; b) comparing the expression level of IL18BP to a predetermined cutoff value; and c) identifying absence of ABMR in the subject when the expression level of IL18BP is greater than or equal to the predetermined cutoff value.

The present disclosure provides methods of identifying the absence of ABMR in a subject that has undergone a kidney transplant, the method comprising: a) determining the expression level of at least one of five biomarkers in microvesicular RNA and cell-free DNA isolated from a biological sample from the subject, wherein the five biomarkers comprise IL18BP, CXCL11, CD74, CD44 and C3; b) inputting the expression levels from step (a) into an algorithm to generate a score; c) identifying absence of ABMR in the subject based on the score. In some aspects, identifying absence of ABMR in the subject based on a score can comprise comparing the score to a predetermined cutoff value; and determining absence of ABMR based on the relationship between the score and the predetermined cutoff value (e.g. is the score greater than, greater than or equal to, less than, or less than or equal to the predetermined cutoff value).

In a non-limiting example, the present disclosure provides methods of identifying the absence of ABMR in a subject that has undergone a kidney transplant, the method comprising: a) determining the expression level of IL18BP in microvesicular RNA and cell-free DNA isolated from a biological sample from the subject; b) inputting the expression level of IL18BP into an algorithm to generate a score; c) identifying absence of ABMR in the subject based on the score.

The present disclosure provides methods of identifying that a subject that has undergone a kidney transplant is at low risk of having ABMR, the method comprising: a) determining the expression level of at least one of five biomarkers in microvesicular RNA and cell-free DNA isolated from a biological sample from the subject, wherein the five biomarkers comprise IL18BP, CXCL11, CD74, CD44 and C3; and b) identifying that the subject is at low risk of having ABMR based on the expression levels measured in step (a). In some aspects, identifying that the subject is at low risk of having ABMR based on the expression levels measured in step (a) can comprise comparing the one or more expression levels to corresponding predetermined cutoff values and determining that the subject is at low risk of having ABMR based on the relationship between the one or more expression levels and the corresponding predetermined cutoff values (e.g. greater than, greater than or equal to, less than, less than or equal to, or equal to).

The present disclosure provides methods of identifying that a subject that has undergone a kidney transplant is at high risk of having ABMR, the method comprising: a) determining the expression level of at least one of five biomarkers in microvesicular RNA and cell-free DNA isolated from a biological sample from the subject, wherein the five biomarkers comprise IL18BP, CXCL11, CD74, CD44 and C3; and b) identifying that the subject is at high risk of having ABMR based on the expression levels measured in step (a). In some aspects, identifying that the subject is at high risk of having ABMR based on the expression levels measured in step (a) can comprise comparing the one or more expression levels to corresponding predetermined cutoff values and determining that the subject is at high risk of having ABMR based on the relationship between the one or more expression levels and the corresponding predetermined cutoff values (e.g. greater than, greater than or equal to, less than, less than or equal to, or equal to).

The present disclosure provides methods of identifying the risk of ABMR in a subject that has undergone a kidney transplant, the method comprising: a) determining the expression level of at least one of five biomarkers in microvesicular RNA and cell-free DNA isolated from a biological sample from the subject, wherein the five biomarkers comprise IL18BP, CXCL11, CD74, CD44 and C3; and b) identifying the risk of ABMR in the subject based on the expression levels measured in step (a). In some aspects, identifying the risk of ABMR in a subject based on the expression levels measured in step (a) can comprise comparing the one or more expression levels to corresponding predetermined cutoff values and determining the risk based on the relationship between the one or more expression levels and the corresponding predetermined cutoff values (e.g. greater than, greater than or equal to, less than, less than or equal to, or equal to).

In a non-limiting example, the present disclosure provides methods of identifying that a subject that has undergone a kidney transplant is at low risk of having ABMR, the method comprising: a) determining the expression level of IL18BP in microvesicular RNA and cell-free DNA isolated from a biological sample from the subject; b) comparing the expression level of IL18BP to a predetermined cutoff value; and c) identifying that the subject is at low risk of having ABMR based on the relationship between the expression level of IL18BP and the predetermined cutoff value.

In a non-limiting example, the present disclosure provides methods of identifying that a subject that has undergone a kidney transplant is at low risk of having ABMR, the method comprising: a) determining the expression level of IL18BP in microvesicular RNA and cell-free DNA isolated from a biological sample from the subject; b) comparing the expression level of IL18BP to a predetermined cutoff value; and c) identifying that the subject is at low risk of having ABMR when the expression level of IL18BP is greater than or equal to predetermined cutoff value.

In a non-limiting example, the present disclosure provides methods of identifying that a subject that has undergone a kidney transplant is at high risk of having ABMR, the method comprising: a) determining the expression level of IL18BP in microvesicular RNA and cell-free DNA isolated from a biological sample from the subject; b) comparing the expression level of IL18BP to a predetermined cutoff value; and c) identifying that the subject is at high risk of having ABMR based on the relationship between the expression level of IL18BP and the predetermined cutoff value.

In a non-limiting example, the present disclosure provides methods of identifying the risk of ABMR in a subject that has undergone a kidney transplant, the method comprising: a) determining the expression level of IL18BP in microvesicular RNA and cell-free DNA isolated from a biological sample from the subject; b) comparing the expression level of IL18BP to a predetermined cutoff value; and c) identifying the risk of ABMR in a subject based on the relationship between the expression level of IL18BP and the predetermined cutoff value.

The present disclosure provides methods of identifying that a subject that has undergone a kidney transplant is at low risk of having ABMR, the method comprising: a) determining the expression level of at least one of five biomarkers in microvesicular RNA and cell-free DNA isolated from a biological sample from the subject, wherein the five biomarkers comprise IL18BP, CXCL11, CD74, CD44 and C3; b) inputting the expression levels from step (a) into an algorithm to generate a score; c) determining that the subject is at low risk of having ABMR based on the score. In some aspects, identifying that the subject is at low risk of having ABMR based on a score can comprise comparing the score to a predetermined cutoff value; and determining that the subject is at low risk of having ABMR based on the relationship between the score and the predetermined cutoff value (e.g. is the score greater than, greater than or equal to, less than, or less than or equal to the predetermined cutoff value).

The present disclosure provides methods of identifying that a subject that has undergone a kidney transplant is at high risk of having ABMR, the method comprising: a) determining the expression level of at least one of five biomarkers in microvesicular RNA and cell-free DNA isolated from a biological sample from the subject, wherein the five biomarkers comprise IL18BP, CXCL11, CD74, CD44 and C3; b) inputting the expression levels from step (a) into an algorithm to generate a score; c) determining that the subject is at high risk of having ABMR based on the score. In some aspects, identifying that the subject is at high risk of having ABMR based on a score can comprise comparing the score to a predetermined cutoff value; and determining that the subject is at high risk of having ABMR based on the relationship between the score and the predetermined cutoff value (e.g. is the score greater than, greater than or equal to, less than, or less than or equal to the predetermined cutoff value).

The present disclosure provides methods of identifying the risk of ABMR in a subject that has undergone a kidney transplant, the method comprising: a) determining the expression level of at least one of five biomarkers in microvesicular RNA and cell-free DNA isolated from a biological sample from the subject, wherein the five biomarkers comprise IL18BP, CXCL11, CD74, CD44 and C3; b) inputting the expression levels from step (a) into an algorithm to generate a score; c) determining the risk of ABMR in the subject based on the score. In some aspects, identifying the risk of ABMR based on a score can comprise comparing the score to a predetermined cutoff value; and determining the risk based on the relationship between the score and the predetermined cutoff value (e.g. is the score greater than, greater than or equal to, less than, or less than or equal to the predetermined cutoff value).

In a non-limiting example, the present disclosure provides methods of identifying that a subject that has undergone a kidney transplant is at low risk of having ABMR, the method comprising: a) determining the expression level of IL18BP in microvesicular RNA and cell-free DNA isolated from a biological sample from the subject; b) inputting the expression level of IL18BP into an algorithm to generate a score; c) determining that the subject is at low risk of having ABMR based on the score.

In a non-limiting example, the present disclosure provides methods of identifying that a subject that has undergone a kidney transplant is at high risk of having ABMR, the method comprising: a) determining the expression level of IL18BP in microvesicular RNA and cell-free DNA isolated from a biological sample from the subject; b) inputting the expression level of IL18BP into an algorithm to generate a score; c) determining that the subject is at high risk of having ABMR based on the score.

In a non-limiting example, the present disclosure provides methods of identifying the risk of ABMR a subject that has undergone a kidney transplant, the method comprising: a) determining the expression level of IL18BP in microvesicular RNA and cell-free DNA isolated from a biological sample from the subject; b) inputting the expression level of IL18BP into an algorithm to generate a score; c) determining the risk of ABMR in the based on the score.

In any of the preceding methods, the expression levels of IL18BP, CXCL11, CD74, CD44 and/or C3 can be normalized to the expression level of at least one endogenous control gene, and the normalized expression levels can be subsequently analyzed/input into the algorithms to generate a score. That is, wherever expression levels are used in the preceding methods, normalized expression levels can be used.

In some aspects of the methods of the present disclosure, a predetermined cutoff value can be selected as to be optimized to rule out ABMR. In a non-limiting example, such a predetermined cutoff value could have a high negative predictive value.

In some aspects of the methods of the present disclosure, an algorithm can be the product of a feature selection wrapper algorithm and a trained classifier built from at least one predictive classification algorithm. The feature selection wrapper algorithm can be Boruta and the at least one predicative classification algorithm can be SVM-radial.

In some aspects of the methods of the present disclosure, an algorithm can a product of a feature selection wrapper algorithm, machine learning algorithm, trained classifier, logistic regression model or any combination thereof, that was trained using: a) the expression levels of the at least one, or the at least two, or the at least three, or the at least four or each of the biomarkers in at least one biological sample from at least one subject who has ABMR; and b) the expression levels of the at least one, or the at least two, or the at least three, or the at least four or each of the biomarkers in at least one biological sample from at least one subject who has TCMR. In some aspects, the at least one subject who has ABMR is determined to have ABMR based on kidney transplant biopsy results. In some aspects, the at least one subject who has TCMR is determined to have TCMR based on kidney transplant biopsy results.

In some aspects of the methods of the present disclosure, an algorithm can a product of a feature selection wrapper algorithm, machine learning algorithm, trained classifier, logistic regression model or any combination thereof, that was trained using: a) the expression levels of the at least one, or the at least two, or the at least three, or the at least four or each of the biomarkers in at least one biological sample from at least one subject who has ABMR; b) the expression levels of the at least one, or the at least two, or the at least three, or the at least four or each of the biomarkers in at least one biological sample from at least one subject who has TCMR; and c) the expression levels of the at least one, or the at least two, or the at least three, or the at least four or each of the biomarkers in at least one biological sample from at least one subject who has TCMR and ABMR. In some aspects, the at least one subject who has ABMR is determined to have ABMR based on kidney transplant biopsy results. In some aspects, the at least one subject who has TCMR is determined to have TCMR based on kidney transplant biopsy results.

In some aspects of the preceding methods, an algorithm and a corresponding predetermined cutoff value can have, or be selected to have, a sensitivity for ruling out ABMR of at least about 70%. In some aspects of the preceding methods, an algorithm and a corresponding predetermined cutoff value can have, or be selected to have, a sensitivity for ruling out ABMR of at least about 77%.

In some aspects of the preceding methods, an algorithm and a corresponding predetermined cutoff value can have, or be selected to have, a specificity for ruling out ABMR of at least about 60%. In some aspects of the preceding methods, an algorithm and a corresponding predetermined cutoff value can have, or be selected to have, a specificity for ruling out ABMR of at least about 62%.

In some aspects of the preceding methods, an algorithm and a corresponding predetermined cutoff value can have, or be selected to have, a positive predictive value (PPV) for ruling out ABMR of at least about 30%. In some aspects of the preceding methods, an algorithm and a corresponding predetermined cutoff value can have, or be selected to have, a PPV for ruling out ABMR of at least about 38%.

In some aspects of the preceding methods, an algorithm and a corresponding predetermined cutoff value can have, or be selected to have, a negative predictive value (NPV) for ruling out ABMR of at least about 80%. In some aspects of the preceding methods, an algorithm and a corresponding predetermined cutoff value can have, or be selected to have, a NPV for ruling out ABMR of at least about 90%.

In some aspects of the preceding methods, an algorithm and a corresponding predetermined cutoff value can have, or be selected to have, a sensitivity for identifying kidney transplant rejection of at least about 60%. In some aspects of the preceding methods, an algorithm and a corresponding predetermined cutoff value can have, or be selected to have, a sensitivity for identifying kidney transplant rejection of at least about 61%.

In some aspects of the preceding methods, an algorithm and a corresponding predetermined cutoff value can have, or be selected to have, a specificity for identifying kidney transplant rejection of at least about 80%. In some aspects of the preceding methods, an algorithm and a corresponding predetermined cutoff value can have, or be selected to have, a specificity for identifying kidney transplant rejection of at least about 84%.

In some aspects of the preceding methods, an algorithm and a corresponding predetermined cutoff value can have, or be selected to have, a positive predictive value (PPV) for identifying kidney transplant rejection of at least about 50%.

In some aspects of the preceding methods, an algorithm and a corresponding predetermined cutoff value can have, or be selected to have, a negative predictive value (NPV) for identifying kidney transplant rejection of at least about 90%.

### General Methods and Definitions

Any of the following general methods and definitions can be applied to any of the classifiers described above.

In some aspects of the preceding methods, the subject can be previously identified as having a kidney transplant rejection and/or being at high risk of a kidney transplant rejection using any method known in the art, including, but not limited to, biopsy analysis and/or any of the methods put forth in PCT Patent Publication No. WO 2021/243206.

As would be appreciated by the skilled artisan, the phrases “clinically indicated biopsy” and “for-cause biopsy” are used herein interchangeably to describe biopsies that are prescribed to subjects who have undergone a kidney transplant and who are presenting with at least one clinical indication of kidney transplant rejection, including, but not limited to, increased serum creatine levels.

As would be appreciated by the skilled artisan, the phrases “protocol biopsy” and “management biopsy” are used herein interchangeably to describe biopsies that are prescribed to subjects who have undergone a kidney transplant but who are otherwise not presenting with a clinical indication of kidney transplant rejection, in that the biopsy is implemented as a pre-emptive measure to identify any signs of transplant rejection.

In some aspects, any method of the present disclosure, prior to step (a), can further comprise: i) isolating a plurality of microvesicles from a biological sample from the subject; and ii) extracting at least one microvesicular RNA from the plurality of isolated microvesicles.

In some aspects, any method of the present disclosure, prior to step (a), can further comprise: i) isolating a microvesicle fraction from a biological sample from the subject, wherein the microvesicle fraction comprises a plurality of microvesicles and cfDNA; ii) extracting at least one microvesicular RNA and at least one cfDNA molecule from the plurality of isolated microvesicles.

In some aspects of the methods of the present disclosure, isolating a plurality of microvesicles from a biological sample from the subject can comprise a processing step to remove cells, cellular debris or a combination of cells and cellular debris. A processing step can comprise filtering the sample, centrifuging the sample, or a combination of filtering the sample and centrifuging the sample. Centrifuging can comprise centrifuging at about 2000×g. Filtering can comprise filtering the sample through a filter with a pore size of about 0.8 microns. Filtering can comprise filtering the sample through a filter with a pore size of about 5 microns. Filtering can comprise filtering a sample through a filter with a pore size of about 0.8 microns to about 5 microns. Filtering can comprise filtering the sample through a filter with a pore size of up to about 5 microns.

In some aspects of the methods of the present disclosure, isolating a plurality of microvesicles can comprise ultrafiltration, ultracentrifugation, ion-exchange chromatography, size exclusion chromatography, density gradient centrifugation, centrifugation, differential centrifugation, immunoabsorbent capture, affinity purification, affinity exclusion, microfluidic separation, nanomembrane concentration or any combination thereof.

In some aspects of the methods of the present disclosure, isolating a microvesicle fraction, wherein the microvesicle fraction comprises a plurality of microvesicles can comprise ultrafiltration, ultracentrifugation, ion-exchange chromatography, size exclusion chromatography, density gradient centrifugation, centrifugation, differential centrifugation, immunoabsorbent capture, affinity purification, affinity exclusion, microfluidic separation, nanomembrane concentration or any combination thereof.

In some aspects of the methods of the present disclosure, isolating an at least one microvesicle is from a bodily fluid sample can comprise contacting the bodily fluid sample with at least one affinity agent that binds to at least one surface marker present on the surface the at least one microvesicle.

Other microvesicle and microvesicle fraction isolation procedures are described in US 2017-0088898 A1, US 2016-0348095 A1, US 2016-0237422 A1, US 2015-0353920 A1, U.S. Pat. No. 10,465,183 and US 2019-0284548 A1, the contents of each of which are incorporated herein by reference in their entireties. The methods of the present disclosure can comprise any of the methods described in the aforementioned United States Patent Publications and United States Patents.

In some aspects of the methods of the present disclosure, a biological sample (e.g. a biological sample from the subject and/or a biological sample in the training set) can be a urine sample, a first-catch urine sample or a second voided urine sample. A biological sample can have a volume of between at least about 1 ml to at least about 50 ml. A biological sample can have a volume of up to about 20 ml. A biological sample can have a volume of at least 3 ml. A biological sample can have a volume of about 3 ml to about 10 ml.

In some aspects of the preceding methods, the kidney transplant rejection can be an any-cause kidney transplant rejection.

In some aspects of the preceding methods, the kidney transplant rejection can be T-cell-mediated rejection (TCMR). In some aspects of the preceding methods, the TCMR can be: (a) TCMR Grade IA; (b) TCMR Grade IB; (c) TCMR Grade IIA; (d) TCMR Grade IIB; or (e) TCMR Grade III.

In some aspects of the preceding methods, the kidney transplant rejection is borderline rejection.

In some aspects of the preceding methods, the kidney transplant rejection is antibody-mediated rejection (ABMR). In some aspects of the preceding methods, the ABMR is active ABMR or chronic active ABMR.

In some aspects of the preceding methods, the at least one endogenous control gene can comprise PGK1. An endogenous control gene can also be referred to as a reference biomarker.

In some aspects of the preceding methods, step (a) can further comprise measuring the expression of the at least one endogenous control gene in the nucleic acids (RNA, microvesicular RNA, cell-free DNA, etc.) that are being analyzed.

In some aspects of the methods of the present disclosure, determining the expression level of a biomarker can comprise quantitative PCR (qPCR), quantitative real-time PCR, semi-quantitative real-time PCR, reverse transcription PCR (RT-PCR), reverse transcription quantitative PCR (qRT-PCR), microarray analysis, sequencing, next-generation sequencing (NGS), high-throughput sequencing, direct-analysis, droplet digital PCR, or any combination thereof.

In some aspects of the methods of the present disclosure, an expression level of a biomarker or endogenous control gene can correspond to a cycle threshold (Ct) value when the expression level is determined using quantitative PCR (qPCR), quantitative real-time PCR, semi-quantitative real-time PCR, reverse transcription PCR (RT-PCR) or reverse transcription quantitative PCR (qRT-PCR).

In some aspects, normalizing the expression level of a biomarker to the expression level of an endogenous control gene can comprise subtracting the expression level of the endogenous control gene from the expression level of the biomarker. Accordingly, in aspects wherein the expression levels are measured as Ct values, the normalized expression value of a biomarker can be the Ct value of the biomarker minus the Ct value of the endogenous control gene.

In some aspects of the methods of the present disclosure, a predetermined cutoff value can have, or be selected as to have, a negative predictive value (NPV) of at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%, or at least about 99.9%.

In some aspects of the methods of the present disclosure, a predetermined cutoff value can have, or be selected as to have, a positive predictive value (PPV) of at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%, or at least about 99.9%.

In some aspects of the methods of the present disclosure, a predetermined cutoff value can have, or be selected as to have, a sensitivity of at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%, or at least about 99.9%.

In some aspects of the methods of the present disclosure, a predetermined cutoff value can have, or be selected as to have, a specificity of at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%, or at least about 99.9%.

In some aspects of the methods of the present disclosure, a predetermined cutoff value can be selected as to be optimized to rule-out kidney transplant rejection. Without wishing to be bound by theory, such a predetermined cutoff value would be advantageous in situations where kidney transplant rejection has been clinically indicated (e.g., serum creatinine levels in a subject are rising).

In some aspects of the methods of the present disclosure, a predetermined cutoff value can be selected as to be optimized to rule-in kidney transplant rejection. In a non-limiting example, such a predetermined cutoff value could have a high positive predictive value. Without wishing to be bound by theory, such a predetermined cutoff value would be advantageous in situations where kidney transplant rejection has not been clinically indicated and/or a clinician is determining whether to proceed with renal biopsy and/or kidney transplant rejection therapy.

In some aspects of the methods of the present disclosure, a predetermined cutoff value can be calculated and/or selected using at least one receiver operating characteristic (ROC) curve. In some aspects of the methods of the present disclosure, a predetermined cutoff value can be calculated and/or selected to have any of the features described herein (e.g., a specific sensitivity, specificity, PPV, NPV or any combination thereof) using any method known in the art, as would be appreciated by the skilled artisan.

In some aspects of the methods of the present disclosure, an algorithm can be the product of a feature selection wrapper algorithm. In some aspects of the methods of the present disclosure, an algorithm can be the product of a machine learning algorithm. In some aspects of the methods of the present disclosure, an algorithm can be the product of a trained classifier built from at least one predictive classification algorithm. In some aspects, an algorithm can be the product of a feature selection wrapper algorithm, a machine learning algorithm, a trained classifier built from at least one predictive classification algorithm or any combination thereof. In some aspects of the methods of the present disclosure, an algorithm can be the product of a feature selection wrapper algorithm and a trained classifier built from at least one predictive classification algorithm.

In some aspects of the methods of the present disclosure, an algorithm can be the product of a feature selection wrapper algorithm and a trained classifier built from at least one predictive classification algorithm.

In some aspects of the methods of the present disclosure a predictive classification algorithm, a feature selection wrapper algorithm, and/or a machine learning algorithm can comprise XGBoost (XGB), random forest (RF), Lasso and Elastic-Net Regularized Generalized Linear Models (glmnet), Linear Discriminant Analysis (LDA), cforest, classification and regression tree (CART), treebag, k nearest-neighbor (knn), neural network (nnet), support vector machine-radial (SVM-radial), support vector machine-linear (SVM-linear), naïve Bayes (NB), multilayer perceptron (mlp), Boruta (see Kursa M B, Rudnicki W R. Feature Selection with the Boruta Package. J Stat Softw 2010; 36(11), incorporated herein by reference in its entirety) or any combination thereof.

In some aspects of the methods of the present disclosure, an algorithm can be the product of a feature selection wrapper algorithm. In some aspects of the methods of the present disclosure, an algorithm can be the product of a machine learning algorithm. In some aspects of the methods of the present disclosure, an algorithm can be the product of a trained classifier built from at least one predictive classification algorithm. In some aspects of the methods of the present disclosure, an algorithm can be the product of a of a logistic regression model. A logistic regression model can comprise LASSO regularization. In some aspects, an algorithm can be the product of a feature selection wrapper algorithm, a machine learning algorithm, a trained classifier built from at least one predictive classification algorithm or any combination thereof. In some aspects of the methods of the present disclosure, an algorithm can be the product of a feature selection wrapper algorithm and a trained classifier built from at least one predictive classification algorithm.

In some aspects of the methods of the present disclosure a predictive classification algorithm, a feature selection wrapper algorithm, and/or a machine learning algorithm can comprise XGBoost (XGB), random forest (RF), Lasso and Elastic-Net Regularized Generalized Linear Models (glmnet), cforest, classification and regression tree (CART), treebag, k nearest-neighbor (knn), neural network (nnet), support vector machine-radial (SVM-radial), support vector machine-linear (SVM-linear), naïve bayes (NB), multilayer perceptron (mlp), Boruta (see Kursa M B, Rudnicki W R. Feature Selection with the Boruta Package. J Stat Softw 2010; 36(11), incorporated herein by reference in its entirety) or any combination thereof.

In some aspects of the methods of the present disclosure, an algorithm can be a product of a feature selection wrapper algorithm, machine learning algorithm, trained classifier, logistic regression model or any combination thereof, that was trained to identify kidney transplant rejection in a subject using: a) the expression levels of the at least two, or the at least three, or the at least four, or the at least five, or the at least six, or the at least seven in at least one biological sample from at least one subject who is kidney transplant rejection negative; and b) the expression levels of the at least two, or the at least three, or the at least four, or the at least five, or the at least six, or the at least seven in at least one biological sample from at least one subject who is kidney transplant rejection positive. In some aspects, the at least one subject who is kidney transplant rejection negative is determined to be kidney transplant rejection negative based on kidney transplant biopsy results. In some aspects, the at least one subject who is kidney transplant rejection positive is determined to be kidney transplant rejection positive based on kidney transplant biopsy results.

In some aspects of the preceding methods, the trained classifier is trained using the expression levels of the biomarkers measured in the microvesicular RNA isolated from a training set of biological samples, wherein the training set of biological sample comprises a first plurality of biological samples isolated from subjects identified to be at risk for a kidney transplant rejection based on at least one clinical indications of kidney transplant rejection.

The methods of the present disclosure can further comprise performing a kidney biopsy on a subject identified as being at risk for a kidney transplant rejection.

The methods of the present disclosure can further comprise administering at least one kidney transplant rejection therapy to a subject identified as being at risk for a kidney transplant rejection.

The methods of the present disclosure can further comprise administering at least one kidney transplant rejection therapy to a subject identified as having a kidney transplant rejection.

In some aspects of the methods of the present disclosure, an at least one kidney transplant rejection therapy can comprise administering to the subject at least one therapeutically effective amount of at least one immunosuppressant, at least one therapeutically effective amount of at least one steroid, at least one therapeutically effective amount of at least one corticosteroid, at least one therapeutically effective amount of at least one steroid, at least one therapeutically effective amount of at least one anti-T-cell antibody, at least one therapeutically effective amount of mycophenolate mofetil (MMF), at least one therapeutically effective amount of cyclosporine A (CsA), at least one therapeutically effective amount of tacrolimus, at least one therapeutically effective amount of azathioprine, at least one therapeutically effective amount of muromonab (OKT-3), at least one therapeutically effective amount of anti-thymocyte globulin (ATG), at least one therapeutically effective amount of anti-lymphocyte globulin (ALG), at least one therapeutically effective amount of Campath (alemtuzumab), at least one therapeutically effective amount of prednisone, at least one therapeutically effective amount of mycophenolic acid, at least one therapeutically effective amount of rapamycin, at least one therapeutically effective amount of belatacept, at least one therapeutically effective amount of intravenous immunoglobulin (IVIg), at least one therapeutically effective amount of an anti-CD20 agent (e.g. rituximab), at least one therapeutically effective amount of bortezomib or any combination thereof.

In some aspects, an at least one kidney transplant rejection therapy can comprise performing plasmapheresis.

In some aspects, a therapeutically effective amount of at least one steroid comprises a high dose regimen of the at least one steroid.

In some aspects, a therapeutically effective amount of at least one corticosteroid comprises a high dose regimen of the at least one steroid.

Any of the preceding methods can comprise administering at least one kidney transplant rejection therapy to a subject identified as having TCMR or ABMR. In some aspects, any of the preceding methods can comprise administering at least one TCMR-targeted therapy to a subject identified as having TCMR and/or administering at least one ABMR-targeted therapy to a subject identified as having ABMR.

Any of the preceding methods can further comprise administering at least one TCMR-targeted therapy to a subject identified as having an absence of ABMR.

Any of the preceding methods can further comprise administering at least one TCMR-targeted therapy to a subject identified as having a low risk of ABMR.

In some aspects, a TCMR-targeted therapy can comprise administering to the subject at least one therapeutically effective amount of at least one steroid, at least one therapeutically effective amount of at least one corticosteroid, at least one therapeutically effective amount of muromonab (OKT-3), at least one therapeutically effective amount of anti-thymocyte globulin (ATG), at least one therapeutically effective amount of Campath (alemtuzumab), at least one therapeutically effective amount of prednisone, at least one therapeutically effective amount of tacrolimus, at least one therapeutically effective amount of cyclosporine A, at least one therapeutically effective amount of mycophenolic acid, at least one therapeutically effective amount of azathioprine, at least one therapeutically effective amount of rapamycin, at least one therapeutically effective amount of belatacept, or any combination thereof.

In some aspects, a TCMR-targeted therapy can comprise administering to the subject at least one therapeutically effective amount of at least one steroid, at least one therapeutically effective amount of at least one corticosteroid, at least one therapeutically effective amount of muromonab (OKT-3), at least one therapeutically effective amount of anti-thymocyte globulin (ATG), at least one therapeutically effective amount of Campath (alemtuzumab), or any combination thereof.

The methods of the present disclosure can further comprise optimizing existing maintenance therapy that a subject is undergoing when the subject is identified as having TCMR. In some aspects, the maintenance therapy can comprise the administration of prednisone, tacrolimus, cyclosporine A, mycophenolic acid, azathioprine, rapamycin, belatacept or any combination thereof.

In some aspects, an ABMR-targeted therapy can comprise administering to the subject at least one therapeutically effective amount of at least one steroid, at least one therapeutically effective amount of at least one corticosteroid, at least one therapeutically effective amount of anti-thymocyte globulin (ATG), at least one therapeutically effective amount of intravenous immunoglobulin (IVIg), at least one therapeutically effective amount of an anti-CD20 agent (e.g. rituximab), at least one therapeutically effective amount of bortezomib, or any combination thereof.

The methods of the present disclosure can further comprise administering at least one kidney inflammation therapy to a subject identified as being at risk for kidney inflammation or identified as having kidney inflammation.

In some aspects, a kidney inflammation therapy can be any kidney inflammation therapy known in the art.

In some aspects, a kidney inflammation therapy can comprise administering at least one immunosuppressant. Non-limiting examples of immunosuppressants include steroids, belatacept, leflunomide, cyclophosphamide, mycophenolate mofetil, azathioprine, ciclosporin, tacrolimus, and cephalosporins.

In some aspects, a kidney inflammation therapy can comprise administering at least one antiviral. Non-limiting examples of antivirals include cidofovir, maribavir, ganciclovir, valganciclovir, valacyclovir, and brincidofovir.

In some aspects, a kidney inflammation therapy can comprise administering at least one antibiotic. Non-limiting examples of antibiotics include quinolone antibiotics, nitrofurantoin, sulfonamides, amoxicillin, trimethoprim/sulfamethoxazole, doxycycline, and quinolones.

In some aspects, a kidney inflammation therapy can comprise administering at least one biologic. Non-limiting examples of biologics include rituximab, belimumab, bortezomib, IVIG, and eculizumab.

In some aspects, a kidney inflammation therapy can comprise reducing an immunosuppression regime that a subject was previously administered.

In some aspects, a kidney inflammation therapy can comprise administering at least one steroid.

In some aspects, a kidney inflammation therapy can comprise administering at least one corticosteroid.

In some aspects, a kidney inflammation therapy can comprise administering at least one blood pressure medication.

In some aspects, a kidney inflammation therapy can comprise effectuating one or more changes in the diet of the subject.

In some aspects, a kidney inflammation therapy can comprise administering at least one diuretic agent.

In some aspects, a kidney inflammation therapy can comprise performing dialysis on a subject.

In some aspects, a kidney inflammation therapy can comprise performing plasmapheresis on a subject.

### EXEMPLARY EMBODIMENTS

Embodiment 1a. A method of determining the risk of a kidney transplant rejection in a subject who has undergone a kidney transplant, the method comprising:
a) determining the expression level of at least two of three biomarkers and at least one endogenous control gene in RNA isolated from a biological sample from the subject,
wherein the three biomarkers comprise IL32, B2M, and CXCL11;
b) normalizing the expression of the biomarkers to the expression level of the at least one endogenous control gene;
c) inputting the normalized expression levels from step (b) into an algorithm to generate a score; and
d) determining the risk of a kidney transplant rejection in the subject based on the score.

Embodiment 1b. A method of determining the risk of a kidney transplant rejection in a subject who has undergone a kidney transplant, the method comprising:
a) determining the expression level of at least two of three biomarkers and at least one endogenous control gene in microvesicular RNA isolated from a biological sample from the subject,
wherein the three biomarkers comprise IL32, B2M, and CXCL11;
b) normalizing the expression of the biomarkers to the expression level of the at least one endogenous control gene;
c) inputting the normalized expression levels from step (b) into an algorithm to generate a score; and
d) determining the risk of a kidney transplant rejection in the subject based on the score.

Embodiment 2. The method of embodiment 1a or embodiment 1b, wherein the subject is a subject who has been identified to be at risk for a kidney transplant rejection based on at least one clinical indications of kidney transplant rejection.

Embodiment 3. The method of embodiment 2, wherein the at least one clinical indication of kidney transplant rejection comprises increased serum creatinine.

Embodiment 4. The method of any one of the preceding embodiments, wherein step (a) comprises determining the expression level of each of the three biomarkers.

Embodiment 5. The method of any one of the preceding embodiments, wherein determining the risk of a kidney transplant rejection in the subject based on the score comprises:
i) comparing the score to a predetermined cutoff value; and
ii) determining that the at the subject is at a high risk of having a kidney transplant rejection when the score is greater than or equal to the predetermined cutoff value or determining that the subject is at low risk of having a kidney transplant rejection when the score is less than the predetermined cutoff value.

Embodiment 6. The method of embodiment 5, wherein the algorithm is the product of a feature selection wrapper algorithm, a machine learning algorithm, a trained classifier built from at least one predictive classification algorithm or any combination thereof.

Embodiment 7. The method of embodiment 6, wherein the predictive classification algorithm, the feature selection wrapper algorithm, and/or the machine learning algorithm comprises XGBoost (XGB), random forest (RF), Lasso and Elastic-Net Regularized Generalized Linear Models (glmnet), Linear Discriminant Analysis (LDA), cforest, classification and regression tree (CART), treebag, k nearest-neighbor (knn), neural network (nnet), support vector machine-radial (SVM-radial), support vector machine-linear (SVM-linear), naïve Bayes (NB), multilayer perceptron (mlp), Boruta or any combination thereof.

Embodiment 8. The method of embodiment 5, wherein the algorithm is the product of a trained classifier built from at least one predictive classification algorithm, wherein the at least one predictive classification algorithm comprises SVM-linear.

Embodiment 9a. The method of embodiment 8, wherein the trained classifier is trained using the expression levels of the biomarkers measured in RNA isolated from a training set of biological samples, wherein the training set of biological sample comprises:
i) a first plurality of biological samples isolated from subjects identified to be at risk for a kidney transplant rejection based on at least one clinical indications of kidney transplant rejection.

Embodiment 9b. The method of embodiment 8, wherein the trained classifier is trained using the expression levels of the biomarkers measured in microvesicular RNA isolated from a training set of biological samples, wherein the training set of biological sample comprises:
i) a first plurality of biological samples isolated from subjects identified to be at risk for a kidney transplant rejection based on at least one clinical indications of kidney transplant rejection.

Embodiment 10. The method of embodiment 9a or embodiment 9b, wherein the at least one clinical indication of kidney transplant rejection comprises increased serum creatinine.

Embodiment 11. The method of embodiment 9a, embodiment 9b or embodiment 10, wherein
a portion of the biological samples in the first plurality of biological samples are from subjects who are identified by biopsy to be positive for kidney transplant rejection, and
a portion of the biological samples in the first plurality of biological samples are from subjects who are identified by biopsy to be negative for kidney transplant rejection.

Embodiment 12. The method of any one of embodiments 9a-11, wherein the training set of biological samples further comprises:
ii) a second plurality of biological samples isolated from subjects that have no clinical indications of a kidney transplant rejection

Embodiment 13. The method of embodiment 12, wherein the biological samples in the second plurality are from subjects identified by biopsy to be negative for kidney transplant rejection.

Embodiment 14. The method of any one of embodiments 8-13, wherein the algorithm and the predetermined cutoff value has a sensitivity for identifying kidney transplant rejection of at least about 90%.

Embodiment 15. The method of any one of embodiments 8-14, wherein the algorithm and the predetermined cutoff value has a sensitivity for identifying kidney transplant rejection of at least about 93%.

Embodiment 16. The method of any one of embodiments 8-15, wherein the algorithm and the predetermined cutoff value has a specificity for identifying kidney transplant rejection of at least about 40%.

Embodiment 17. The method of any one of embodiments 8-16, wherein the algorithm and the predetermined cutoff value has a specificity for identifying kidney transplant rejection of at least about 43%.

Embodiment 18. The method of any one of embodiments 8-17, wherein the algorithm and the predetermined cutoff value has a PPV for identifying kidney transplant rejection of at least about 40%.

Embodiment 19. The method of any one of embodiments 8-18, wherein the algorithm and the predetermined cutoff value has a PPV for identifying kidney transplant rejection of at least about 45%.

Embodiment 20. The method of any one of embodiments 8-19, wherein the algorithm and the predetermined cutoff value has a NPV for identifying kidney transplant rejection of at least about 90%.

Embodiment 21. The method of any one of embodiments 8-20, wherein the algorithm and the predetermined cutoff value has a NPV for identifying kidney transplant rejection of at least about 93%.

Embodiment 22. The method of any one of embodiments 8-21, wherein the algorithm and the predetermined cutoff value has
i) a sensitivity for identifying kidney transplant rejection of at least about 90%; and
ii) a negative predictive value for identifying kidney transplant rejection of at least about 90%.

Embodiment 23. The method of any one of embodiments 8-22, wherein the algorithm and the predetermined cutoff value has
i) a sensitivity for identifying kidney transplant rejection of at least about 90%; and
ii) a negative predictive value for identifying kidney transplant rejection of at least about 90%;
iii) a specificity for identifying kidney transplant rejection of at least about 40%; and
iv) a positive predictive value for identifying kidney transplant rejection of at least about 40%.

Embodiment 24. The method of any one of embodiments 8-23, wherein the algorithm and the predetermined cutoff value has
i) a sensitivity for identifying kidney transplant rejection of at least about 93%; and
ii) a negative predictive value for identifying kidney transplant rejection of at least about 93%.

Embodiment 25. The method of any one of embodiments 8-24, wherein the algorithm and the predetermined cutoff value has
i) a sensitivity for identifying kidney transplant rejection of at least about 93%; and
ii) a negative predictive value for identifying kidney transplant rejection of at least about 93%;
iii) a specificity for identifying kidney transplant rejection of at least about 43%; and
iv) a positive predictive value for identifying kidney transplant rejection of at least about 45%.

Embodiment 26a. A method of determining the risk of a kidney transplant rejection in a subject who has undergone a kidney transplant, the method comprising:
a) determining the expression level of at least two of six biomarkers and at least one endogenous control gene in RNA isolated from a biological sample from the subject,
wherein the six biomarkers comprise IFNAR2, PYCARD, CD44, IRAK2, B2M, and NAMPT;
b) normalizing the expression of the biomarkers to the expression level of the at least one endogenous control gene;
c) inputting the normalized expression levels from step (b) into an algorithm to generate a score; and
d) determining the risk of a kidney transplant rejection in the subject based on the score.

Embodiment 26b. A method of determining the risk of a kidney transplant rejection in a subject who has undergone a kidney transplant, the method comprising:
a) determining the expression level of at least two of six biomarkers and at least one endogenous control gene in microvesicular RNA isolated from a biological sample from the subject,
wherein the six biomarkers comprise IFNAR2, PYCARD, CD44, IRAK2, B2M, and NAMPT;
b) normalizing the expression of the biomarkers to the expression level of the at least one endogenous control gene;
c) inputting the normalized expression levels from step (b) into an algorithm to generate a score; and
d) determining the risk of a kidney transplant rejection in the subject based on the score.

Embodiment 27. The method of embodiment 26a or embodiment 26b, wherein the subject is a subject that has no clinical indications of a kidney transplant rejection.

Embodiment 28. The method of embodiment 26a, embodiment 26b or embodiment 27, wherein step (a) comprises determining the expression level of:
a) at least three of the six biomarkers;
b) at least four of the six biomarkers; or
c) at least five of the six biomarkers.

Embodiment 29. The method of any one of embodiments 26a-28, wherein step (a) comprises determining the expression level of each of the six biomarkers.

Embodiment 30. The method of any one of embodiments 26a-29, wherein determining the risk of a kidney transplant rejection in the subject based on the score comprises:
i) comparing the score to a predetermined cutoff value; and
ii) determining that the at the subject is at a high risk of having a kidney transplant rejection when the score is greater than or equal to the predetermined cutoff value or determining that the subject is at low risk of having a kidney transplant rejection when the score is less than the predetermined cutoff value.

Embodiment 31. The method of embodiment 30, wherein the algorithm is the product of a feature selection wrapper algorithm, a machine learning algorithm, a trained classifier built from at least one predictive classification algorithm or any combination thereof.

Embodiment 32. The method of embodiment 31, wherein the predictive classification algorithm, the feature selection wrapper algorithm, and/or the machine learning algorithm comprises XGBoost (XGB), random forest (RF), Lasso and Elastic-Net Regularized Generalized Linear Models (glmnet), Linear Discriminant Analysis (LDA), cforest, classification and regression tree (CART), treebag, k nearest-neighbor (knn), neural network (nnet), support vector machine-radial (SVM-radial), support vector machine-linear (SVM-linear), naïve Bayes (NB), multilayer perceptron (mlp), Boruta or any combination thereof.

Embodiment 33. The method of embodiment 30, wherein the algorithm is the product of:
a first trained classifier built from at least one predictive classification algorithm, wherein the at least one predictive classification algorithm comprises naïve Bayes; and
an at least second trained classifier built from at least one predictive classification algorithm, wherein the at least one predictive classification algorithm comprises naïve Bayes.

Embodiment 34b. The method of embodiment 33, wherein the first and/or the at least second trained classifier(s) is/are trained using the expression levels of the biomarkers measured in RNA isolated from a training set of biological samples, wherein the training set of biological sample comprises:
i) a first plurality of biological samples isolated from subjects that have no clinical indications of a kidney transplant rejection.

Embodiment 34b. The method of embodiment 33, wherein the first and/or the at least second trained classifier(s) is/are trained using the expression levels of the biomarkers measured in microvesicular RNA isolated from a training set of biological samples, wherein the training set of biological sample comprises:
i) a first plurality of biological samples isolated from subjects that have no clinical indications of a kidney transplant rejection.

Embodiment 35. The method of embodiment 34a or embodiment 34b, wherein
a portion of the biological samples in the first plurality of biological samples are from subjects who are identified by biopsy to be positive for kidney transplant rejection, and
a portion of the biological samples in the first plurality of biological samples are from subjects who are identified by biopsy to be negative for kidney transplant rejection.

Embodiment 36. The method of embodiment 34a, embodiment 34b, or embodiment 35, wherein the training set of biological samples further comprises:
ii) a second plurality of biological samples isolated from subjects identified to be at risk for a kidney transplant rejection based on at least one clinical indications of kidney transplant rejection.

Embodiment 37. The method of embodiment 36, wherein the biological samples in the second plurality are from subjects identified by biopsy to be positive for kidney transplant rejection.

Embodiment 38. The method of any one of embodiments 33-37, wherein the first trained classifier has a sensitivity for identifying kidney transplant rejection of at least about 90%.

Embodiment 39. The method of any one of embodiments 33-38, wherein the at least second trained classifier has a specificity for identifying kidney transplant rejection of at least about 90%.

Embodiment 40. The method of any one of embodiments 33-39, wherein the algorithm and the predetermined cutoff value has a sensitivity for identifying kidney transplant rejection of at least about 90%.

Embodiment 41. The method of any one of embodiments 33-40, wherein the algorithm and the predetermined cutoff value has a sensitivity for identifying kidney transplant rejection of at least about 93%.

Embodiment 42. The method of any one of embodiments 33-41, wherein the algorithm and the predetermined cutoff value has a specificity for identifying kidney transplant rejection of at least about 40%.

Embodiment 43. The method of any one of embodiments 33-42, wherein the algorithm and the predetermined cutoff value has a specificity for identifying kidney transplant rejection of at least about 48%.

Embodiment 44. The method of any one of embodiments 33-43, wherein the algorithm and the predetermined cutoff value has a PPV for identifying kidney transplant rejection of at least about 30%.

Embodiment 45. The method of any one of embodiments 33-44, wherein the algorithm and the predetermined cutoff value has a PPV for identifying kidney transplant rejection of at least about 32%.

Embodiment 46. The method of any one of embodiments 33-45, wherein the algorithm and the predetermined cutoff value has a NPV for identifying kidney transplant rejection of at least about 90%.

Embodiment 47. The method of any one of embodiments 33-46, wherein the algorithm and the predetermined cutoff value has a NPV for identifying kidney transplant rejection of at least about 97%.

Embodiment 48. The method of any one of embodiments 33-47, wherein the algorithm and the predetermined cutoff value has
i) a sensitivity for identifying kidney transplant rejection of at least about 90%;
ii) a specificity for identifying kidney transplant rejection of at least about 40%; and
iii) a negative predictive value for identifying kidney transplant rejection of at least about 90%.

Embodiment 49. The method of any one of embodiments 33-48, wherein the algorithm and the predetermined cutoff value has
i) a sensitivity for identifying kidney transplant rejection of at least about 93%;
ii) a specificity for identifying kidney transplant rejection of at least about 48%; and
iii) a negative predictive value for identifying kidney transplant rejection of at least about 97%.

Embodiment 50. The method of any one of embodiments 33-49, wherein the algorithm and the predetermined cutoff value has
i) a sensitivity for identifying kidney transplant rejection of at least about 90%;
ii) a specificity for identifying kidney transplant rejection of at least about 40%;
iii) a negative predictive value for identifying kidney transplant rejection of at least about 90%; and
iv) a positive predictive value for identifying kidney transplant rejection of at least about 30%.

Embodiment 51. The method of any one of embodiments 33-50, wherein the algorithm and the predetermined cutoff value has
i) a sensitivity for identifying kidney transplant rejection of at least about 93%;
ii) a specificity for identifying kidney transplant rejection of at least about 48%;
iii) a negative predictive value for identifying kidney transplant rejection of at least about 97%; and
iv) a positive predictive value for identifying kidney transplant rejection of at least about 32%.

Embodiment 52. The method of any one of embodiments 33-51, wherein the algorithm and the predetermined cutoff value has a sensitivity for identifying kidney transplant rejection of at least about 60%.

Embodiment 53. The method of any one of embodiments 33-52, wherein the algorithm and the predetermined cutoff value has a sensitivity for identifying kidney transplant rejection of at least about 61%.

Embodiment 54. The method of any one of embodiments 33-53, wherein the algorithm and the predetermined cutoff value has a specificity for identifying kidney transplant rejection of at least about 80%.

Embodiment 55. The method of any one of embodiments 33-54, wherein the algorithm and the predetermined cutoff value has a specificity for identifying kidney transplant rejection of at least about 84%.

Embodiment 56. The method of any one of embodiments 33-55, wherein the algorithm and the predetermined cutoff value has a PPV for identifying kidney transplant rejection of at least about 50%.

Embodiment 57. The method of any one of embodiments 33-56, wherein the algorithm and the predetermined cutoff value has a NPV for identifying kidney transplant rejection of at least about 90%.

Embodiment 58. The method of any one of embodiments 33-57, wherein the algorithm and the predetermined cutoff value has
i) a sensitivity for identifying kidney transplant rejection of at least about 60%;
ii) a specificity for identifying kidney transplant rejection of at least about 80%; and
iii) a negative predictive value for identifying kidney transplant rejection of at least about 90%.

Embodiment 59. The method of any one of embodiments 33-58, wherein the algorithm and the predetermined cutoff value has
i) a sensitivity for identifying kidney transplant rejection of at least about 61%;
ii) a specificity for identifying kidney transplant rejection of at least about 84%; and
iii) a negative predictive value for identifying kidney transplant rejection of at least about 90%.

Embodiment 60. The method of any one of embodiments 33-59, wherein the algorithm and the predetermined cutoff value has
i) a sensitivity for identifying kidney transplant rejection of at least about 60%;
ii) a specificity for identifying kidney transplant rejection of at least about 80%; and
iii) a negative predictive value for identifying kidney transplant rejection of at least about 90%; and
iv) a positive predictive value for identifying kidney transplant rejection of at least about 50%.

Embodiment 61. The method of any one of embodiments 33-60, wherein the algorithm and the predetermined cutoff value has
i) a sensitivity for identifying kidney transplant rejection of at least about 61%;
ii) a specificity for identifying kidney transplant rejection of at least about 84%; and
iii) a negative predictive value for identifying kidney transplant rejection of at least about 90%; and
iv) a positive predictive value for identifying kidney transplant rejection of at least about 50%.

Embodiment 62. The method of any one of the preceding embodiments, wherein the kidney transplant rejection is any-cause kidney transplant rejection.

Embodiment 63. The method of embodiment 62, wherein the kidney transplant rejection is T-cell-mediated rejection (TCMR).

Embodiment 64. The method of embodiment 63, wherein the TCMR is:
(a) TCMR Grade IA;
(b) TCMR Grade IB;
(c) TCMR Grade IIA;
(d) TCMR Grade IIB; or
(e) TCMR Grade III.

Embodiment 65. The method of embodiment 62, wherein the kidney transplant rejection is borderline rejection.

Embodiment 66. The method of embodiment 62, wherein the kidney transplant rejection is antibody-mediated rejection (ABMR).

Embodiment 67. The method of embodiment 66, wherein the ABMR is active ABMR or chronic active ABMR.

Embodiment 68. The method of any one of the preceding embodiments, wherein the biological sample from the subject and/or the biological samples in the training sets is/are urine samples.

Embodiment 69. The method of embodiment 68, wherein the urine sample(s) comprise
a) first-catch urine sample; or
b) second voided urine.

Embodiment 70. The method of any one of the preceding embodiments, wherein the biological sample has a volume of between at least about 1 ml to at least about 50 ml, preferably wherein the biological sample has a volume of about 3 ml to about 10 ml.

Embodiment 71. The method of any one of the preceding embodiments, wherein the at least one endogenous control gene comprises PGK1.

Embodiment 72. The method of any one of the preceding embodiments, wherein determining the expression level of a biomarker comprises quantitative PCR (qPCR), quantitative real-time PCR, semi-quantitative real-time PCR, reverse transcription PCR (RT-PCR), reverse transcription quantitative PCR (qRT-PCR), microarray analysis, sequencing, next-generation sequencing (NGS), high-throughput sequencing, direct-analysis, droplet digital PCR, or any combination thereof.

Embodiment 73. The method of any one of the preceding embodiments, further comprising performing a kidney biopsy on a subject identified as being at risk for a kidney transplant rejection.

Embodiment 74. The method of any one of the preceding embodiments, further comprising administering to a subject identified as being at risk for a kidney transplant rejection at least one kidney transplant rejection therapy.

Embodiment 75. The method of embodiment 74, wherein the at least one kidney transplant rejection therapy comprises administering to the subject at least one therapeutically effective amount of at least one immunosuppressant, at least one steroid, at least one corticosteroid, at least one anti-T-cell antibody, mycophenolate mofetil (MMF), cyclosporine A (CsA), tacrolimus, azathioprine, muromonab (OKT-3), anti-thymocyte globulin (ATG), anti-lymphocyte globulin (ALG), Campath (alemtuzumab), prednisone, mycophenolic acid, rapamycin, belatacept, intravenous immunoglobulin (IVIg), an anti-CD20 agent, rituximab, bortezomib, or any combination thereof.

Embodiment 76. The method of any one of the preceding embodiments, wherein the RNA isolated from a biological sample from the subject comprises cell-free RNA.

Embodiment 77. The method of any one of the preceding embodiments, wherein the RNA isolated from a biological sample from the subject comprises microvesicular RNA.

Embodiment 78. The method of any one of the preceding embodiments, wherein the RNA isolated from a training set of biological samples comprises cell-free RNA.

Embodiment 79. The method of any one of the preceding embodiments, wherein the RNA isolated from a training set of biological samples comprises microvesicular RNA.

Embodiment 80. A method of determining the absence of antibody-mediated rejection (ABMR) in a subject that has undergone a kidney transplant, the method comprising.
a) determining the expression level of at least two of five biomarkers in microvesicular RNA isolated from a biological sample from the subject, wherein the five biomarkers comprise IL18BP, CXCL11, CD74, CD44 and C3;
b) inputting the expression levels from step (a) into an algorithm to generate a score;
c) identifying the absence of ABMR in the subject based on the score.

Embodiment 81. The method of embodiment 80, wherein step (c) comprises:
i) comparing the score to a predetermined cutoff value; and
ii) identifying the absence of ABMR in the subject when the score is less than or equal to a predetermined cutoff value.

Embodiment 82. A method of determining the risk of antibody-mediated rejection (ABMR) in a subject that has undergone a kidney transplant, the method comprising:
a) determining the expression level of at least two of five biomarkers in microvesicular RNA isolated from a biological sample from the subject, wherein the five biomarkers comprise IL18BP, CXCL11, CD74, CD44 and C3;
b) inputting the expression levels from step (a) into an algorithm to generate a score;
c) identifying the risk of ABMR in the subject based on the score.

Embodiment 83. The method of embodiment 82, wherein step (c) comprises:
i) comparing the score to a predetermined cutoff value; and
ii) identifying the risk of ABMR in the subject based on relationship between the score and the predetermined cutoff value.

Embodiment 84. A method of determining that a subject that has undergone a kidney transplant is at low risk of ABMR, the method comprising:
a) determining the expression level of at least two of five biomarkers in microvesicular RNA isolated from a biological sample from the subject, wherein the five biomarkers comprise IL18BP, CXCL11, CD74, CD44 and C3;
b) inputting the expression levels from step (a) into an algorithm to generate a score;
c) identifying that the subject is at low risk of having ABMR based on the score.

Embodiment 85. The method of embodiment 84, wherein step (c) comprises:
i) comparing the score to a predetermined cutoff value; and
ii) identifying that the subject is at low risk of ABMR when the score is less than or equal to a predetermined cutoff value.

Embodiment 86. A method of determining that a subject that has undergone a kidney transplant is at high risk of ABMR, the method comprising:
a) determining the expression level of at least two of five biomarkers in microvesicular RNA isolated from a biological sample from the subject, wherein the five biomarkers comprise IL18BP, CXCL11, CD74, CD44 and C3;
b) inputting the expression levels from step (a) into an algorithm to generate a score;
c) identifying that the subject is at high risk of having ABMR based on the score.

Embodiment 87. The method of embodiment 86, wherein step (c) comprises:
i) comparing the score to a predetermined cutoff value; and
ii) identifying that the subject is at high risk of ABMR based on the relationship between the score and the predetermined cutoff value.

Embodiment 88. The method of any one of the preceding embodiments, wherein the subject is a subject who has been identified as having a kidney transplant rejection or who has been identified as being at high risk for kidney transplant rejection.

Embodiment 89. The method of any one of the preceding embodiments, wherein step (a) comprises determining the expression level of each of the five biomarkers.

Embodiment 90. The method of any one of the preceding embodiments, wherein the algorithm is the product of a feature selection wrapper algorithm, a machine learning algorithm, a trained classifier built from at least one predictive classification algorithm or any combination thereof.

Embodiment 91. The method of embodiment 90, wherein the predictive classification algorithm, the feature selection wrapper algorithm, and/or the machine learning algorithm comprises XGBoost (XGB), random forest (RF), Lasso and Elastic-Net Regularized Generalized Linear Models (glmnet), Linear Discriminant Analysis (LDA), cforest, classification and regression tree (CART), treebag, k nearest-neighbor (knn), neural network (nnet), support vector machine-radial (SVM-radial), support vector machine-linear (SVM-linear), naïve Bayes (NB), multilayer perceptron (mlp), Boruta or any combination thereof.

Embodiment 92. The method of embodiment 90 or 91, wherein the algorithm is a product of a feature selection wrapper algorithm, machine learning algorithm, trained classifier, logistic regression model or any combination thereof, that was trained using: a) the expression levels of the at least one, or the at least two, or the at least three, or the at least four or each of the biomarkers in at least one biological sample from at least one subject who has ABMR; and b) the expression levels of the at least one, or the at least two, or the at least three, or the at least four or each of the biomarkers in at least one biological sample from at least one subject who has TCMR.

Embodiment 93. The method of any of the preceding embodiments, wherein step (a) further comprises:
(i) determining the expression level of at least one reference biomarker;
(ii) normalizing the expression level of the at least one biomarker to the expression level of the at least one reference biomarker, preferably wherein the at least one reference biomarker comprises PGK1.

Embodiment 94. The method of any of the preceding embodiments, wherein inputting the expression levels from step (a) into an algorithm to generate a score comprises inputting the normalized expression levels from step (a) into an algorithm to generate a score.

Embodiment 95. The method of any of the preceding embodiments, wherein determining the expression level of a biomarker comprises quantitative PCR (qPCR), quantitative real-time PCR, semi-quantitative real-time PCR, reverse transcription PCR (RT-PCR), reverse transcription quantitative PCR (qRT-PCR), digital PCR (dPCR), microarray analysis, sequencing, next-generation sequencing (NGS), high-throughput sequencing, direct-analysis or any combination thereof.

Embodiment 96. The method of any of the preceding embodiments, wherein the predetermined cutoff value has a negative predictive value of at least about 80%, or at least about 85%, or at least about 90%, or at least about 95%, or at least about 99%, or at least about 99.9%.

Embodiment 97. The method of any of the preceding embodiments, wherein the predetermined cutoff value has a positive predictive value of at least about 80%, or at least about 85%, or at least about 90%, or at least about 95%, or at least about 99%, or at least about 99.9%.

Embodiment 98. The method of any of the preceding embodiments, wherein the predetermined cutoff value has a sensitivity of at least about 80%, or at least about 85%, or at least about 90%, or at least about 95%, or at least about 99%, or at least about 99.9%.

Embodiment 99. The method of any of the preceding embodiments, wherein the predetermined cutoff value has a specificity of at least about 80%, or at least about 85%, or at least about 90%, or at least about 95%, or at least about 99%, or at least about 99.9%.

Embodiment 100. The method of any of the preceding embodiments, wherein the predetermined cutoff value is calculated using at least one receiver operating characteristic (ROC) curve.

Embodiment 101. The method of any one of the preceding embodiments, wherein the biological sample comprises urine.

Embodiment 102. The method of embodiment 101, wherein the urine comprises:
a) first-catch urine sample; and/or
b) second voided urine.

Embodiment 103. The method of any one of the preceding embodiments, wherein the biological sample has a volume of between at least about 1 ml to at least about 50 ml, preferably wherein the biological sample has a volume of about 3 ml to about 10 ml.

Embodiment 104. The method of any one of the preceding embodiments, further comprising administering to a subject identified as not having ABMR or identified as having a low risk of ABMR at least one TCMR-targeted therapy.

Embodiment 105. The method of embodiment 104, wherein the at least one TCMR-targeted therapy comprises administering to the subject at least one therapeutically effective amount of at least one steroid, at least one therapeutically effective amount of at least one corticosteroid, at least one therapeutically effective amount of muromonab (OKT-3), at least one therapeutically effective amount of anti-thymocyte globulin (ATG), at least one therapeutically effective amount of Campath (alemtuzumab), at least one therapeutically effective amount of prednisone, at least one therapeutically effective amount of tacrolimus, at least one therapeutically effective amount of cyclosporine A, at least one therapeutically effective amount of mycophenolic acid, at least one therapeutically effective amount of azathioprine, at least one therapeutically effective amount of rapamycin, at least one therapeutically effective amount of belatacept, or any combination thereof.

### EXAMPLES

### Example 1

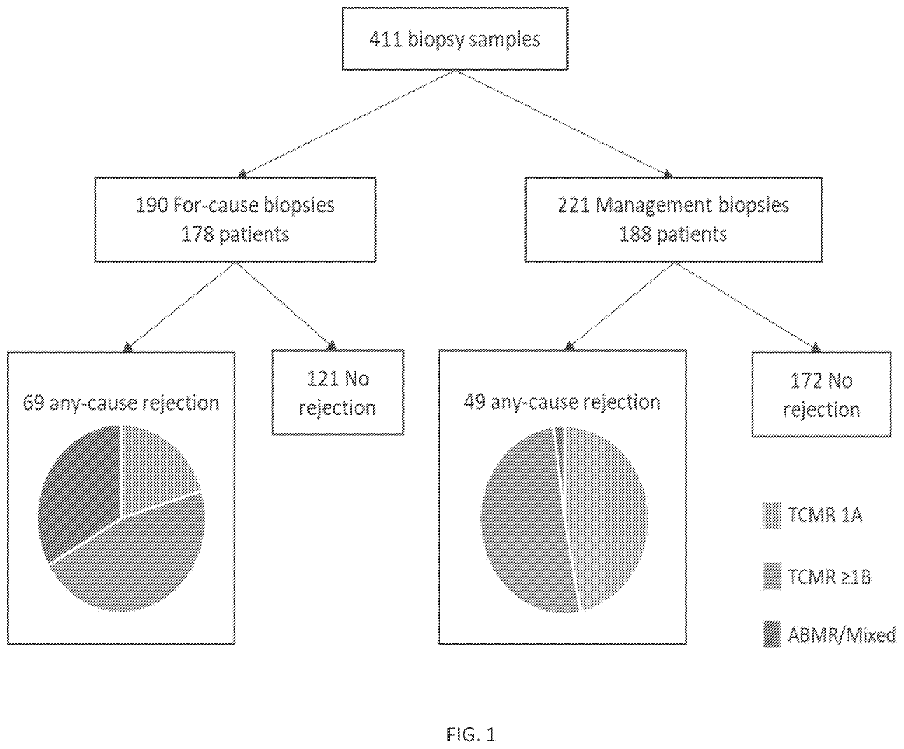

The following non-limiting example describes a study of 411 urine samples with matched biopsy specimens collected from 366 renal transplant patients used to facilitate urinary microvesicular mRNA profiling and derive the gene signatures used in the kidney transplant rejection identification methods described herein. Of these 411 urine samples, 190 urine samples were associated with clinically indicated (for-cause) biopsies and 221 urine samples were associated with management (protocol) biopsies. Of the 190 samples associated with for-cause biopsies, 121 samples were determined to be kidney rejection negative by biopsy and 69 were kidney rejection positive. Of the 69 rejection positive, 23 were antibody-mediated kidney transplant rejection (ABMR) positive and 46 were cell-mediated kidney transplant rejection (TCMR) positive. Of the 46 cell-mediated rejection positive, 14 were categorized as Banff 1A and 32 were categorized as Banff ≥1B. Eight of the 69 rejection positive samples were annotated as borderline. Of the 221 urine samples associated with a protocol biopsy, 172 samples were determined to be kidney rejection negative and 49 were kidney rejection positive. The representation of ABMR, TCMR 1A, and TCMR ≥1B was 1, 23, and 25 respectively. Five of the 49 rejection positive samples were annotated as borderline. A schematic of the samples used is shown in FIG. 1.

### Methods

### Patient and Sample Information

Patients were enrolled in this study across four different renal transplant centers. The study was approved by the institutional review board at each site, and the patients were provided with written informed consent in accord with the Declaration of Helsinki. Patients were enrolled in this study while either presenting with a clinically indicated (for-cause) biopsy or during a routine protocol (management) biopsy.

Demographic clinical characteristics, and information on the donors, were collected from each site's respective electronic health records system. Patient biopsies were evaluated in accordance with each site's established protocols. The renal transplant biopsy specimen pathologists' reports, based on Banff classification were used to discriminate any-cause rejection (including TCMR (Grades IA, IB, IIA, IIB, III), borderline rejection, active ABMR and chronic active ABMR) from no rejection status. Borderline rejection samples were excluded from primary analyses and classifier development. eGFR and ΔeGFR were calculated using the Modification of Diet in Renal Disease equation with standardized serum creatinine values (see Levey, A. S. et al. Expressing the Modification of Diet in Renal Disease Study Equation for Estimating Glomerular Filtration Rate with Standardized Serum Creatinine Values. Clin Chem 53, 766-772, 2007).

Microvesicle Isolation, mRNA Extraction, and Gene-Expression Profiling

After kidney transplantation, urine samples were collected from patients undergoing a transplant kidney biopsy for clinical indications or from patients undergoing a management biopsy.

Voided urine samples were collected within 48 hours of the biopsy, and whole urine samples were stored at −80° C. Samples were thawed and 3-10 ml urine was centrifuged to pellet cells and cellular debris at 2000×g for 20 minutes before the extraction. Exosomal RNA was isolated using a urine-exosome isolation kit (ExoLution RNA). RNA was eluted in 16 μl nuclease-free water (NFW), 14 μl of which was used in a 20 μl reverse transcription reaction using the VILO cDNA synthesis kit (Thermo Fisher).

Target mRNAs were selected based on a 15 gene signature derived to discriminate any-cause rejection and a 5 gene signature derived to differentiate T-cell mediated rejection (TCMR) from antibody mediated rejection (ABMR) that was determined using a for-cause biopsy cohort (see Fekih, R. E. et al. Discovery and Validation of a Urinary Exosome mRNA Signature for the Diagnosis of Human Kidney Transplant Rejection. J Am Soc Nephrol 32, ASN.2020060850, 2021). The union of these two signatures formed a candidate gene set of 17 mRNA targets (B2M, BMP7, C3, CD44, CD74, CXCL11, CXCL14, IFNAR2, IFNGR1, IL18BP, IL32, IRAK2, NAMPT, SERPINA1, STAT1, TBP, and PYCARD) along with the endogenous control gene PGK1.

Specific targets were pre-amplified by adding 12 μl cDNA to a 25 μl reaction containing a primer pool for all 18 targets and TaqMan PreAmp Master mix (Thermo Fisher). After pre-amplification, the reactions were diluted with 100 μl NFW. Two microliters diluted, pre-amplified cDNA was added to 18 different qPCRs, each containing one of the target assays, and TaqMan Fast Universal Master mix (Thermo Fisher). The reactions were loaded onto the QuantStudio 5 Real-Time PCR system (Thermo Fisher) and cycled using the manufacturer's recommended conditions. Ct values were determined using auto baselining and a threshold ΔRn of 0.1.

### Statistics

Raw Ct values for all qPCR assays were filtered for quality control, imputed, and normalized to the endogenous control target, PGK1. Imputation for mRNAs with missing values or Cts that exceed the lower limit of the quantitation of the assay. Limit of detection (LoD) and limit of quantitation (LoQ) were experimentally ascertained for each mRNA assay by way of an extended dilution series. This tested 512 copies to 1 copy of gBlocks Control to determine the LoQ and LoD for each assay. Assay Cts that exceeded the empirically determined assay specific LoQ were imputed as the LoQ Ct. Assay Cts that exceeded the instrument limit of detection (i.e. not reported) were imputed as the LoQ Ct plus one Ct. Finally, each Ct value for the 17 assays was normalized to PGK1 by subtracting the Ct value for PGK1 from each assay's Ct value.

Model, hyperparameter, feature, and threshold selection were performed under 5-fold 2×-repeated cross-validation stratified by patient. Naïve Bayes (NB), logistic regression, support vector machine (SVM), and linear discriminant analysis (LDA) classifiers were evaluated using the Python sklearn module. Univariate features (normalized mRNA Cts) were ranked by AUC and models were evaluated with the top k features, where k ranged from 1 to 7. Bivariate features were analyzed as the second order interaction terms between mRNA pairs (i.e., the product of two normalized mRNA Cts) and ranked by AUC according to an LDA fit. Model thresholds were optimized to achieve a minimum sensitivity or specificity, defined as a parameter of the model selection process. Top performing models were further evaluated under patient stratified leave-one-out cross validation.

An ensemble-learning approach was developed to integrate the scores of a high-sensitivity classifier with scores of a high-specificity classifier. The sub-classifiers are trained and have their thresholds set to achieve >90% sensitivity and specificity for the high-sensitivity and high-specificity sub-classifiers respectively. Samples that are classified as distinctly positive or negative are scored according to their respective sub-classifier. Samples that cannot be confidently classified by either sub-classifier or are equivocally classified are further subjected to score recalibration (weighted averaging of the sub-classifier scores). This process of training the sub-classifiers, setting their thresholds, and optimizing the weighted average of scores is evaluated under cross validation.

Clopper Pearson confidence intervals were calculated for classifier performance metrics including sensitivity, specificity, positive predictive value (PPV), and negative predictive value (NPV). DeLong's test was utilized to determine significance in differences in area under the receiver-operator characteristic curve (AUC) between two classifiers (see DeLong, E. R., DeLong, D. M. & Clarke-Pearson, D. L. Comparing the areas under two or more correlated receiver operating characteristic curves; a nonparametric approach. Biometrics 44, 837-45, 1988). A two-sided Mann-Whitney U rank test was applied to assess for differences in classifier score distributions between non-rejection cases and different rejection subtypes (TCMR 1A, TCMR ≥1B, and ABMR).

### Results

Identifying a Urine Microvesicular RNA Signature for any-Cause Kidney Transplant Rejection in Clinically Indicated (for-Cause) Biopsies

Of the 411 urine samples with matched biopsy specimens, 190 were clinically indicated (for-cause). Of the 190 clinically indicated biopsies, eight were annotated as borderline rejections and were excluded from the primary analysis and classifier development. The normalized RNA expression data for the remaining 182 samples was analyzed to select a classification strategy that distinguishes any-cause rejection from no-rejection cases. To increase the representation of immune quiescent cases during model selection and training, 172 management biopsies that were annotated as no rejection were included. The final model's performance was evaluated on the set of 182 for-cause biopsies.

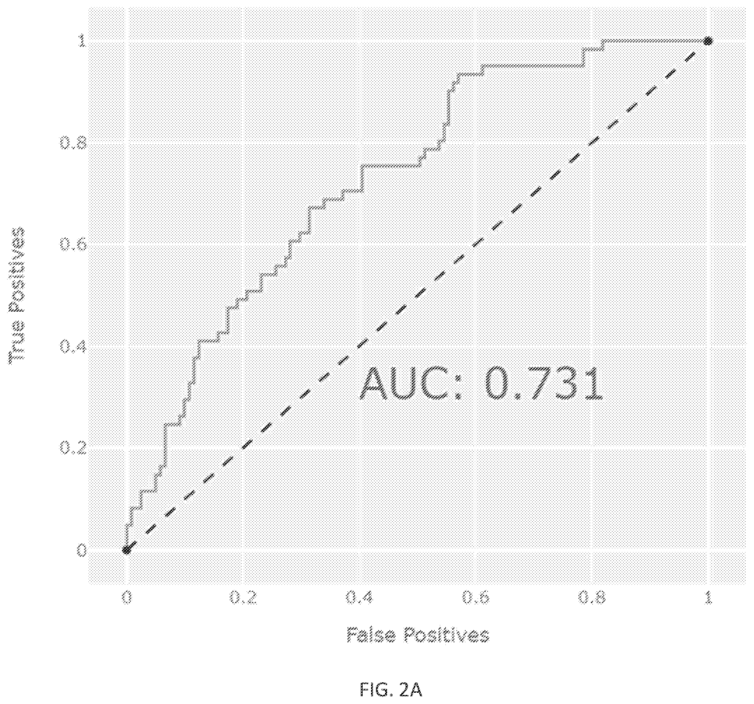

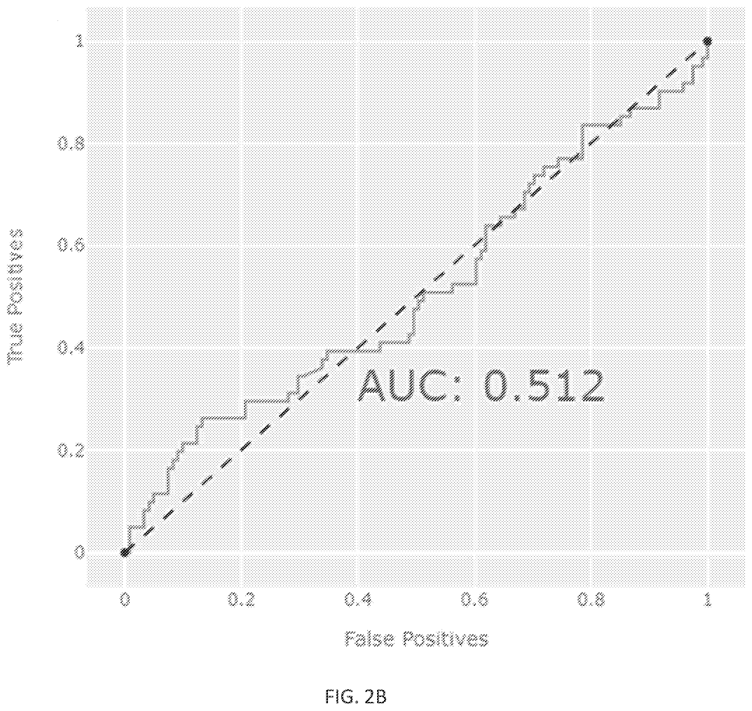

A linear SVM classifier was identified comprised of three univariate mRNA features IL32, B2M, and CXCL11, (used in combination with the endogenous control gene PGK1) that accurately distinguishes any-cause rejection from no rejection according to independent histopathological assessment of concomitant biopsy results. Receiver-operating characteristic (ROC) curve analysis was performed for the 3-gene signature in the set of 182 for-cause biopsies. The area under the curve (AUC) for the linear SVM classifier was 0.731, as shown in FIG. 2A. This method, introduced herein, outperforms the diagnostic predictive performance of the current standard of care for risk management, change in estimated glomerular filtration rate of donor kidney (ΔeGFR), which achieves an AUC of 0.512 (p=0.00048), as shown in FIG. 2B.

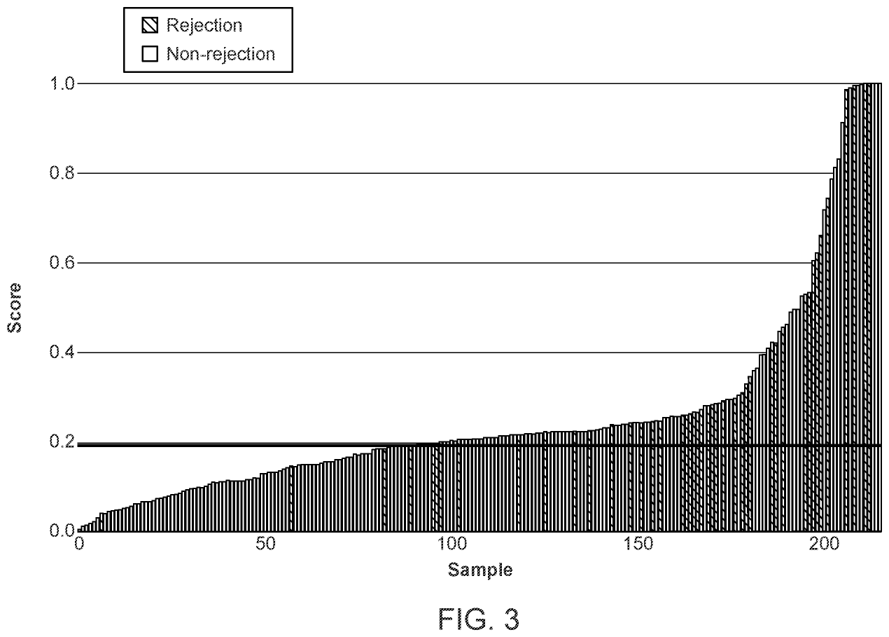

The SVM classifier threshold was optimized to achieve >90% sensitivity, resulting in strong rule-out performance. Under the optimized threshold, the classifier maintained a sensitivity and NPV of 93%, while demonstrating the potential to save 43% of unnecessary biopsies (i.e., correctly classifying samples that were selected for biopsy by clinical indication that were ultimately determined to be rejection negative). These values are presented in Table 1. A cutoff value for the gene signature that optimized both negative predictive value (NPV) and sensitivity in discriminating biopsies with any-cause rejection from those with no rejection was determined, and is shown in in FIG. 3. Only 4 of the 61 samples annotated as “rejection positive” by biopsy were classified as “predicted negative” by this 3-gene signature, as shown in in Table 2. The classifier was applied to the eight borderline cases excluded from the primary analysis, and five of eight were scored as rejection positive.

TABLE 1

| | Point | Confidence |
|---|---|---|
| Metric | Estimate | Interval |
| Sensitivity | 0.93 | (0.841-0.982) |
| Specificity | 0.43 | (0.34-0.523) |
| PPV | 0.45 | (0.364-0.543) |
| NPV | 0.93 | (0.827-0.98) |
| Unnecessary biopsies avoided | 43% | (34%-52%) |

TABLE 2

| | Rejection | Rejection | Predicted |
|---|---|---|---|
| | Positive | Negative | Total |
| Predicted Positive | 57 | 69 | 126 |
| Predicted Negative | 4 | 52 | 56 |
| Total | 61 | 121 | 182 |

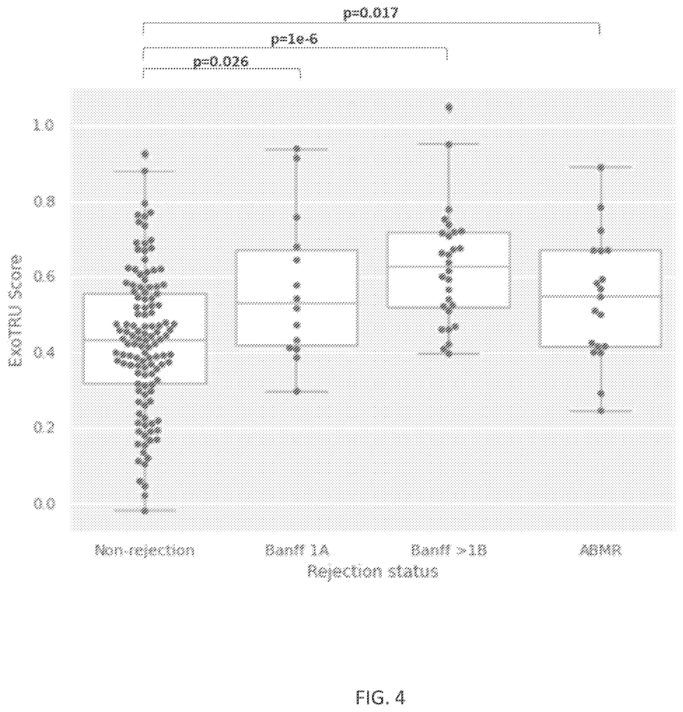

The ability of the classifier to distinguish amongst different types of subclinical rejection was assessed and showed that ABMR (p=0.017), TCMR IA (p=0.026) and TCMR ≥1B (p=1-e6) all scored significantly higher than no rejection cases. These data are presented in FIG. 4. This is notable given that TCMR ≥1B is challenging to detect and TCMR 1A is virtually indistinguishable from non-rejection by existing diagnostic tools such as dd cfDNA, with sensitivities reported at 60% and 0% respectively at a 1% dd cfDNA cutoff (see Bloom, R. D. et al. Cell-Free DNA and Active Rejection in Kidney Allografts. J Am Soc Nephrol 28, 2221-2232, 2017).

These results indicate that the 3-gene signature comprising IL32, B2M, and CXCL11 can be used to identify patients with any-cause kidney transplant rejection and further distinguish and stratify amongst different subtypes of clinical rejection in a method analyzing microvesicular RNA extracted from urinary microvesicles. Moreover, said classifier outperformed the current standard of care for risk management, ΔeGFR and, if deployed in the clinic, would have been able to avoid at least 43% of unnecessary biopsies.

Identifying a Urine Microvesicular RNA Signature for any-Cause Kidney Transplant Rejection in Management Biopsies

Of the total cohort of 411 urine samples, 221 were associated with a management (protocol) biopsy. The mRNA profiles of the candidate markers were analyzed in these management-biopsy associated samples to identify a model capable of distinguishing any-cause rejection from no rejection according to the biopsy result. In the primary analyses and model selection, 5 of the 221 samples with borderline rejection annotations were excluded. To increase the any-cause rejection (in particular, ABMR representation) during model selection and training, 55 for-cause biopsy associated samples that were annotated as TCMR or ABMR were included. The final model's performance was evaluated using the set of 216 management biopsy samples.

Two models with complementary profiles were derived using the samples described above: one with high sensitivity and the other with high specificity. For the rule-out (high sensitivity) model, a naïve Bayes classifier with two bivariate features (IFNAR2|PYCARD and CD44|IRAK2) was identified. A second naïve Bayes classifier comprised of two univariate mRNA features (B2M, NAMPT) that exhibited a strong rule-in profile (high specificity) was also identified. These two models were combined through a weighted ensemble approach to leverage their complementary profiles to generate a six-gene signature comprising IFNAR2, PYCARD, CD44, IRAK2, B2M, and NAMPT. PGK1 was used in combination with this signature as an endogenous control gene.

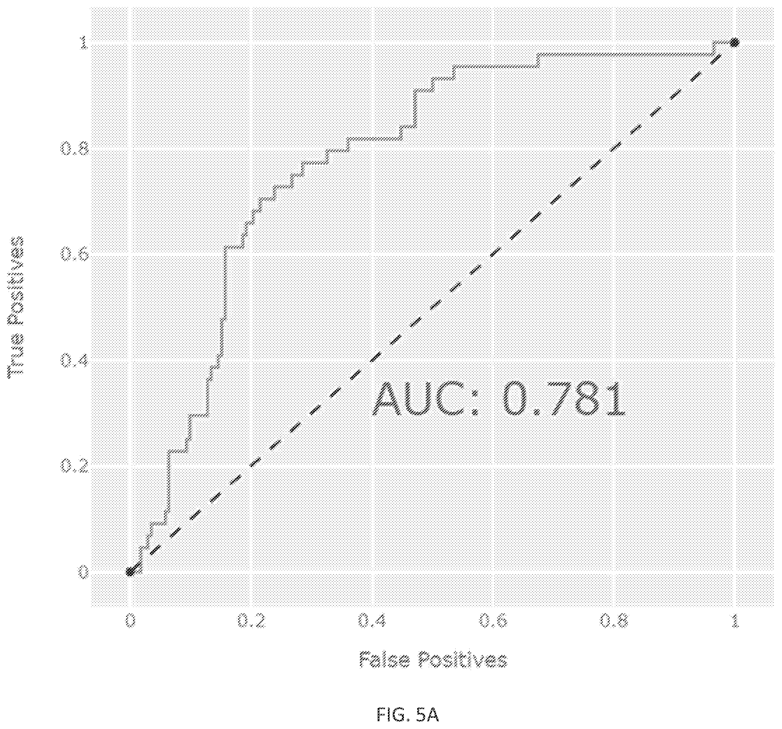

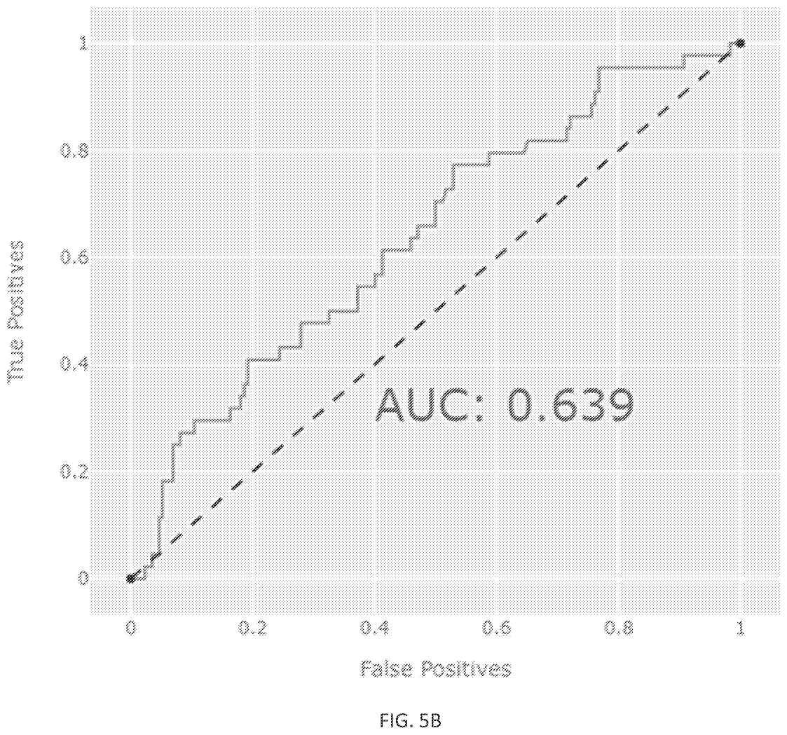

Using the same method as above, ROC curve analysis was performed for the 6-gene signature in the set of 216 management biopsy samples. When evaluated under patient-stratified leave-one-out cross validation, the resulting ensemble classifier achieved an AUC of 0.781 on the primary evaluation cohort 216 management biopsy matched samples. The ROC curve analysis for the ensemble classifier is shown in FIG. 5A. On this management biopsy cohort, the ensemble classifier outperforms the predictive performance of the current standard of care for risk management, ΔeGFR, which has an AUC of 0.639 (p=0.007), as shown in FIG. 5B.

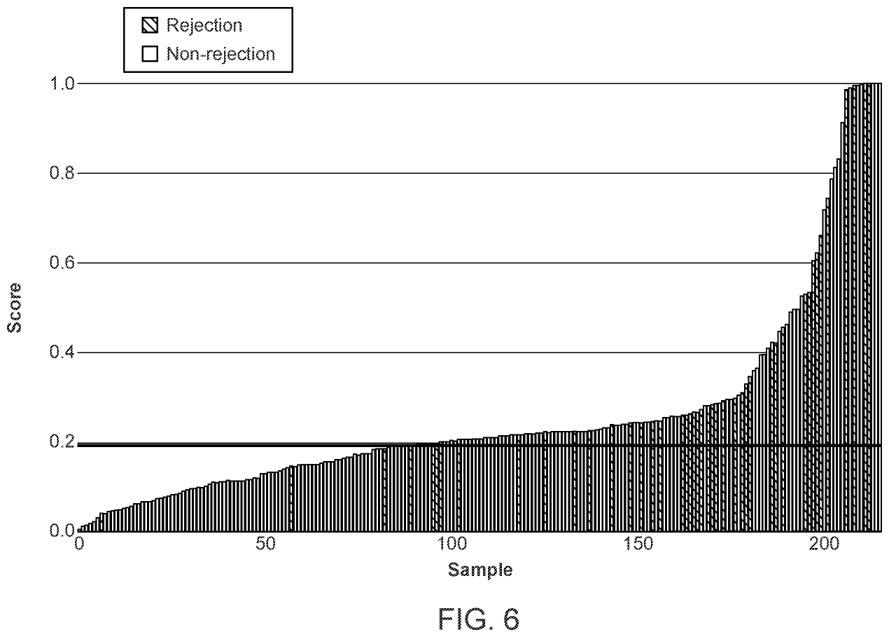

A threshold was derived to rule out any-cause rejection in management biopsy samples with the ensemble classifier, targeting >90% sensitivity. The threshold for the ensemble classifier is depicted in FIG. 6. The resulting classifier and optimized threshold achieved 93% sensitivity and 97% NPV with a specificity of 48%. The performance values for the resulting classifier are shown in Table 3. Of the 44 rejection positive samples, only 3 were misclassified as rejection negative. The rule-out performance values of the classifier are shown in Table 4. A high specificity threshold was evaluated to determine the potential for the ensemble classifier to act as a rule-in diagnostic tool. The classifier's threshold was adjusted to match the reported 84% specificity of 1% dd cfDNA (see Bloom, R. D. et al. Cell-Free DNA and Active Rejection in Kidney Allografts. J Am Soc Nephrol 28, 2221-2232, 2017). The compositionally adjusted sensitivity of 1% dd cfDNA in this management biopsy cohort is estimated to be 29% according to the reported sensitivities of dd cfDNA to ABMR (87.5%), TCMR IA (0%) and TCMR ≥1B (60%). In contrast, the classifier correctly classified the majority of the any-cause rejection cases (achieving 61% sensitivity) at a fixed specificity of 84%. The sensitivity, specificity, PPV and NPV of the classifier are shown in Table 5. The positive and negative predictive values of the classifier are shown in Table 6. This result highlights a distinct performance advantage of the management biopsy classifier relative to existing diagnostic tools. Given the high proportion of TCMR, in particular, low-grade TCMR Banff 1A, the samples used for training are most representative of the first 12 months post-transplant and early-stage rejection (see Halloran, P. F., Reeve, J. P., Pereira, A. B., Hidalgo, L. G. & Famulski, K. S. Antibody-mediated rejection, T cell-mediated rejection, and the injury-repair response: new insights from the Genome Canada studies of kidney transplant biopsies. Kidney Int 85, 258-264, 2014).

TABLE 3

| | Point | Confidence |
|---|---|---|
| Metric | Estimate | Interval |
| Sensitivity | 0.93 | (0.813-0.986) |
| Specificity | 0.48 | (0.406-0.56) |
| PPV | 0.32 | (0.237-0.403) |
| NPV | 0.97 | (0.901-0.993) |
| Unnecessary biopsies avoided | 48% | (34%-52%) |

TABLE 4

| | Positive | Negative | Predicted Total |
|---|---|---|---|
| Predicted Positive | 41 | 89 | 130 |
| Predicted Negative | 3 | 83 | 86 |
| Total | 44 | 172 | 216 |

TABLE 5

| | | Confidence |
|---|---|---|
| Metric | Estimate | Interval |
| Sensitivity | 0.61 | (0.455-0.756) |
| Specificity | 0.84 | (0.78-0.894) |
| PPV | 0.5 | (0.361-0.639) |
| NPV | 0.9 | (0.837-0.938) |

TABLE 6

| | Positive | Negative | Predicted Total |
|---|---|---|---|
| Predicted Positive | 27 | 27 | 54 |
| Predicted Negative | 17 | 145 | 162 |
| Total | 44 | 172 | 216 |

These results indicate that the 6 gene signature comprising IFNAR2, PYCARD, CD44, IRAK2, B2M, and NAMPT can be used to identify patients with any-cause kidney transplant rejection and to stratify both high- and low-risk patients in the kidney transplant surveillance setting enabling earlier detection of subclinical rejection and therapeutic intervention in a method analyzing microvesicular RNA extracted from urinary microvesicles.

### Example 2

The following non-limiting example describes the study of 55 urine samples (a subset of the samples described in Example 1) collected from kidney transplant patients used to derive the gene signatures for the discrimination between ABMR and TCMR described herein. Of the 55 urine samples, 42 were identified as obtained from subjects having TCMR and 13 were identified as obtained from subjects having ABMR.

### Methods

Example 2 was performed according to the methods of Example 1, and methods described herein.

### Results

### Identifying a Rule-Out RNA Signature for ABMR

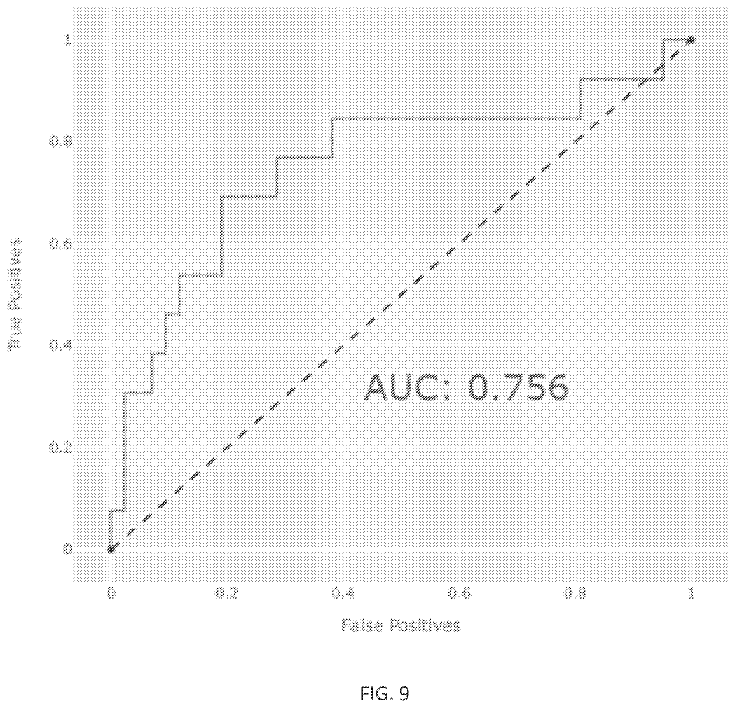

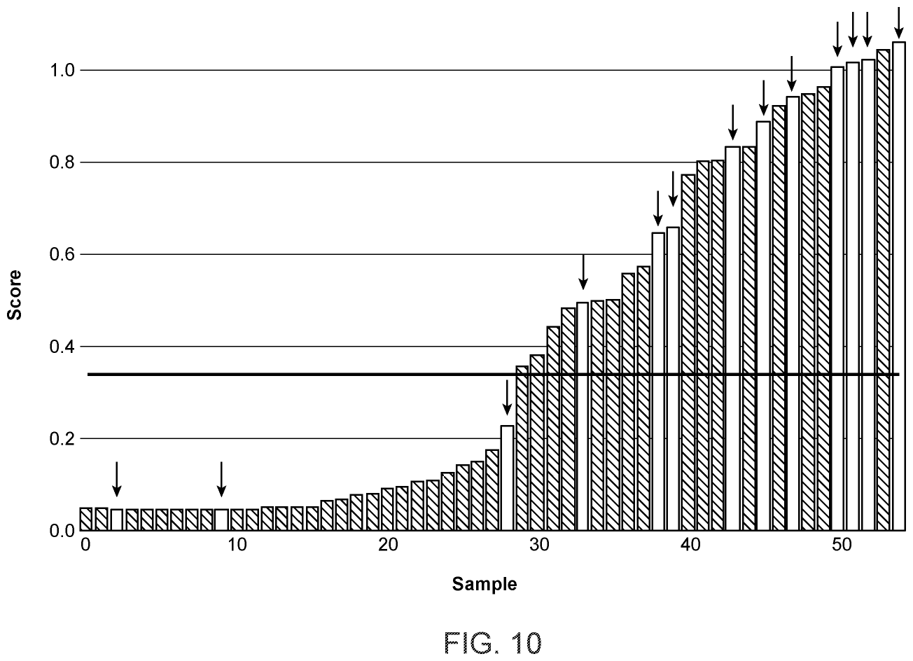

A 5-gene signature comprising IL18BP, CXCL11, CD74, CD44, C3 (used in combination with the endogenous control gene PGK1) that distinguishes TCMR from ABMR was identified. A pre-determined cutoff value was selected to rule out ABMR. Receiver-operating characteristic (ROC) curve analysis was performed for the 5-gene signature and the area under the curve (AUC) was 0.756, as shown in FIG. 9. The 5-gene signature exhibited a 90% NPV (Confidence Interval: 0.726-0.978), and 38% PPV (Confidence Interval: 0.202-0.594), 77% sensitivity (Confidence interval: 0.462-0.95), and 62% specificity (Confidence interval: 0.456-0.764; see FIG. 10). Additionally, the above-analysis indicated that IL18BP is a strong predictor of rejection subtype on its own, exhibiting an AUC value of 0.77 when used alone to rule out ABMR.

### Example 3

The following non-limiting example describes a further study of the samples described in Example 1 to identify a gene signature that can be used to determine if a subject is suffering from or at risk of suffering from kidney inflammation.

### Methods

Example 3 was preformed according to the methods of Example 1, and methods described herein.

### Results

### Assessment of Inflammation and Long-Term Allograft Outcomes in Clinically Indicated Biopsies Initially Negative for Rejection

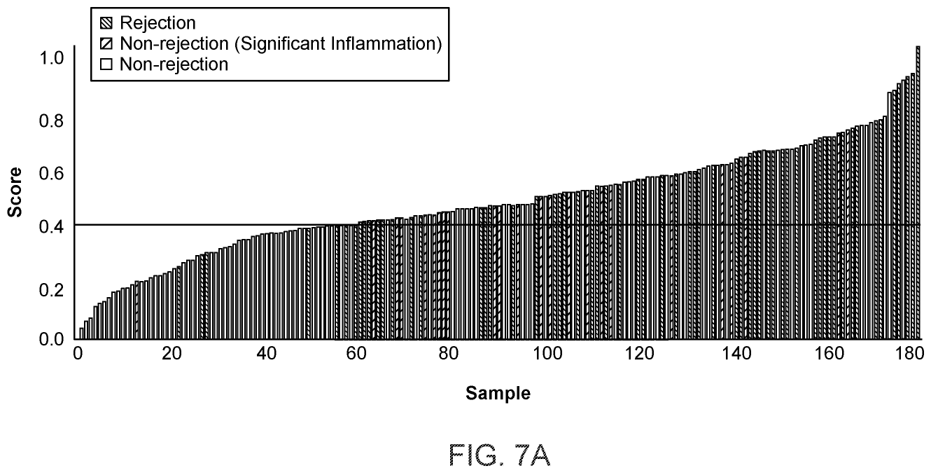

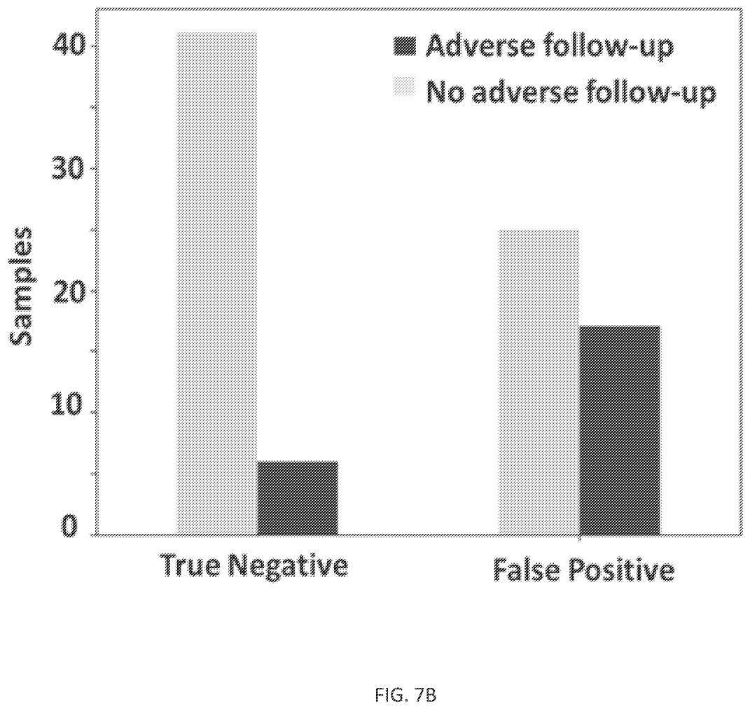

Of the 85 urine samples isolated from subject's with any-cause rejection or significant inflammation, 80 were classified as positive by the SVM classifier comprising three univariate mRNA features IL32, B2M, and CXCL11 (including PGK1 as an endogenous control; see Example 1. When further assessing samples determined to be rejection negative by biopsy, 21/69 (30%) classified as positive by the model (false positive) showed significant inflammation determined by biopsy (FIG. 7A), whereas only one of the 52 rejection-negative samples classified as negative showed significant inflammation (p<0.001, FIG. 7A). Significant inflammation was defined as pathologic lymphoproliferative infiltrate, moderate to severe lymphocytic infiltration, interstitial nephritis (BKV nephritis or acute interstitial nephritis), glomerulopathy, or immune complex deposition. When considering any-cause rejection or significant underlying inflammation as positive, 94% sensitivity, 62% PPV, and 52% specificity was achieved by the model (Table 7).

TABLE 7

| | Any-Cause Rejection | | Any Cause Rejection or | |
|---|---|---|---|---|
| | Only | | Significant Inflammation | |
| | Point | Confidence | Point | Confidence |
| Metric | Estimate | Interval | Estimate | Interval |
| Sensitivity | 0.93 | (0.841-0.982) | 0.94 | (0.865-0.980) |
| Specificity | 0.43 | (0.34-0.523) | 0.52 | (0.413-0.617) |
| PPV | 0.45 | (0.364-0.543) | 0.62 | (0.528-0.704) |
| NPV | 0.93 | (0.827-0.98) | 0.91 | (0.804-0.97) |
| Unnecessary | 43% | (34%-52%) | 52% | (41%-62%) |
| biopsies | | | | |
| avoided | | | | |

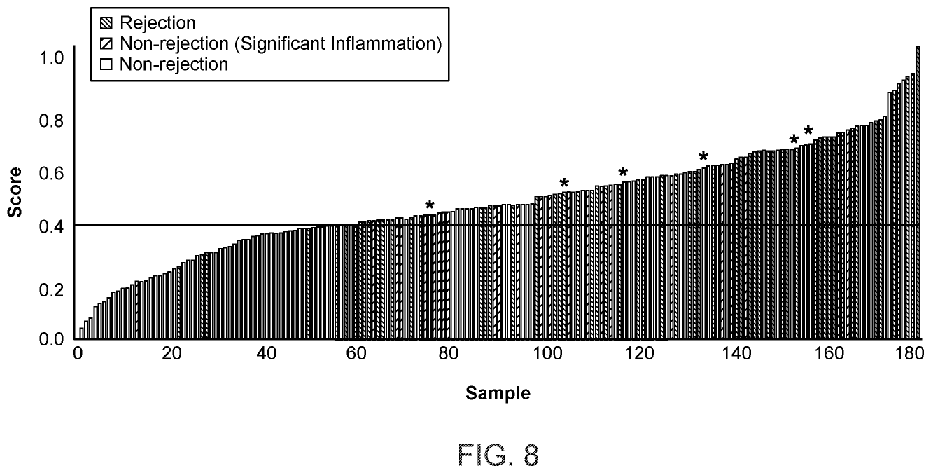

Six patients with false positive samples and no evidence of inflammation on the biopsy developed rejection subsequently. This was documented on repeat clinically indicated biopsies, performed three weeks to six months later, for persistently elevated creatinine (FIG. 8). In the clinically indicated biopsy cohort, three patients with BK viremia and biopsy showing no inflammation were classified as positive by the model. However, two of those three patients had BK nephropathy on a subsequent biopsy performed within three months when their kidney function failed to improve. The third patient developed BK nephritis ten months later. When considering patients that presented with either severe inflammation or rejection at the time of biopsy or in the following ten months as positive cases 95% sensitivity, 68% PPV, and 56% specificity was achieved.

While patients with biopsy proven rejection are known to have worse allograft outcome a cohort of patients with false positive test (high score but biopsy showing no rejection or inflammation) and compared them to patients with true negative signature (low score and biopsy showing no rejection or inflammation) for which follow-up data was available was analyzed. A composite of >30% decrease in eGFR, subsequent rejection or loss of graft with either return to dialysis or re-transplant within the 3 years post biopsy was analyzed. Interestingly, 40.4% of patients with false positive test developed the outcomes within the subsequent 3 years, compared to only 12.7% of patients with true negative test (p=0.003. FIG. 7B). When considering patients that presented with either severe inflammation or rejection at the time of biopsy or in the following ten months or adverse outcomes (described above) within the subsequent 3 years as positive cases 90% sensitivity, 79% PPV, and 62% specificity was achieved.

These results indicate that the 3 gene signature IL32, B2M, and CXCL11 can be used to analyze microvesicular RNA to identify patients having kidney inflammation, thereby allowing for earlier detection of a variety of different inflammation-causing diseases and disorders and for earlier therapeutic intervention.

### EQUIVALENTS

The foregoing description has been presented only for the purposes of illustration and is not intended to limit the disclosure to the precise form disclosed. The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

What is claimed is:

1. A method of determining the risk of a kidney transplant rejection in a subject who has undergone a kidney transplant, the method comprising:
a) determining the expression level of at least two of three biomarkers and at least one endogenous control gene in RNA isolated from a biological sample from the subject,
wherein the three biomarkers comprise IL32, B2M and CXCL11;
b) normalizing the expression of the biomarkers to the expression level of the at least one endogenous control gene;
c) inputting the normalized expression levels from step (b) into an algorithm to generate a score; and
d) determining the risk of a kidney transplant rejection in the subject based on the score.

2. A method of determining the risk of a kidney inflammation in a subject, the method comprising:
a) determining the expression level of at least two of three biomarkers and at least one endogenous control gene in RNA isolated from a biological sample from the subject,
wherein the three biomarkers comprise IL32, B2M and CXCL11;
b) normalizing the expression of the biomarkers to the expression level of the at least one endogenous control gene;
c) inputting the normalized expression levels from step (b) into an algorithm to generate a score; and
d) determining the risk of kidney inflammation in the subject in the subject based on the score.

3. The method of claim 2, wherein the subject has undergone a kidney transplant.

4. The method of any one of the preceding claims, wherein the subject is a subject who has been identified to be at risk for a kidney transplant rejection based on at least one clinical indications of kidney transplant rejection, preferably wherein the at least one clinical indication of kidney transplant rejection comprises increased serum creatinine.

5. The method of any one of the preceding claims, wherein step (a) comprises determining the expression level of each of the three biomarkers.

6. The method of any one of the preceding claims, wherein determining the risk of a kidney transplant rejection in the subject based on the score comprises:
i) comparing the score to a predetermined cutoff value; and
ii) determining that the at the subject is at a high risk of having a kidney transplant rejection when the score is greater than or equal to the predetermined cutoff value or determining that the subject is at low risk of having a kidney transplant rejection when the score is less than the predetermined cutoff value.

7. The method of claim 5 or claim 6, wherein the algorithm is the product of a feature selection wrapper algorithm, a machine learning algorithm, a trained classifier built from at least one predictive classification algorithm or any combination thereof, preferably wherein the algorithm is the product of a trained classifier built from at least one predictive classification algorithm, wherein the at least one predictive classification algorithm comprises SVM-linear.

8. The method of claim 7, wherein the trained classifier is trained using the expression levels of the biomarkers measured in RNA isolated from a training set of biological samples, wherein the training set of biological sample comprises:
i) a first plurality of biological samples isolated from subjects identified to be at risk for a kidney transplant rejection based on at least one clinical indications of kidney transplant rejection, preferably wherein the at least one clinical indication of kidney transplant rejection comprises increased serum creatinine.

9. The method of claim 8, wherein
a portion of the biological samples in the first plurality of biological samples are from subjects who are identified by biopsy to be positive for kidney transplant rejection, and
a portion of the biological samples in the first plurality of biological samples are from subjects who are identified by biopsy to be negative for kidney transplant rejection.

10. The method of claim 8 or claim 9, wherein the training set of biological samples further comprises:
ii) a second plurality of biological samples isolated from subjects that have no clinical indications of a kidney transplant rejection,
preferably wherein the biological samples in the second plurality are from subjects identified by biopsy to be negative for kidney transplant rejection.

11. The method of any one of claims 6-10, wherein the algorithm and the predetermined cutoff value has
i) a sensitivity for identifying kidney transplant rejection of at least about 90%; and
ii) a negative predictive value for identifying kidney transplant rejection of at least about 93%.

12. A method of determining the risk of a kidney transplant rejection in a subject who has undergone a kidney transplant, the method comprising:
a) determining the expression level of at least two of six biomarkers and at least one endogenous control gene in RNA isolated from a biological sample from the subject,
wherein the six biomarkers comprise IFNAR2, PYCARD, CD44, IRAK2, B2M and NAMPT;
b) normalizing the expression of the biomarkers to the expression level of the at least one endogenous control gene;
c) inputting the normalized expression levels from step (b) into an algorithm to generate a score; and
d) determining the risk of a kidney transplant rejection in the subject based on the score.

13. The method of claim 12, wherein the subject is a subject that has no clinical indications of a kidney transplant rejection.

14. The method of claim 12 or claim 13, wherein step (a) comprises determining the expression level of:
a) at least three of the six biomarkers;
b) at least four of the six biomarkers;
c) at least five of the six biomarkers; or
d) each of the six biomarkers.

15. The method of any one of claims 12-14, wherein determining the risk of a kidney transplant rejection in the subject based on the score comprises:
i) comparing the score to a predetermined cutoff value; and
ii) determining that the at the subject is at a high risk of having a kidney transplant rejection when the score is greater than or equal to the predetermined cutoff value or determining that the subject is at low risk of having a kidney transplant rejection when the score is less than the predetermined cutoff value.

16. The method of claim 15, wherein the algorithm is the product of a feature selection wrapper algorithm, a machine learning algorithm, a trained classifier built from at least one predictive classification algorithm or any combination thereof, preferably wherein the algorithm is the product of:
a first trained classifier built from at least one predictive classification algorithm, wherein the at least one predictive classification algorithm comprises naïve Bayes; and
an at least second trained classifier built from at least one predictive classification algorithm, wherein the at least one predictive classification algorithm comprises naïve Bayes.

17. The method of claim 16, wherein the first and/or the at least second trained classifier(s) is/are trained using the expression levels of the biomarkers measured in RNA isolated from a training set of biological samples, wherein the training set of biological sample comprises:
i) a first plurality of biological samples isolated from subjects that have no clinical indications of a kidney transplant rejection.

18. The method of claim 17, wherein
a portion of the biological samples in the first plurality of biological samples are from subjects who are identified by biopsy to be positive for kidney transplant rejection, and
a portion of the biological samples in the first plurality of biological samples are from subjects who are identified by biopsy to be negative for kidney transplant rejection.

19. The method of claim 17 or claim 18, wherein the training set of biological samples further comprises:
ii) a second plurality of biological samples isolated from subjects identified to be at risk for a kidney transplant rejection based on at least one clinical indications of kidney transplant rejection, preferably wherein the biological samples in the second plurality are from subjects identified by biopsy to be positive for kidney transplant rejection.

20. The method of any one of claims 16-19, wherein the first trained classifier has a sensitivity for identifying kidney transplant rejection of at least about 90%.

21. The method of any one of claims 16-20, wherein the at least second trained classifier has a specificity for identifying kidney transplant rejection of at least about 90%.

22. The method of any one of claims 15-21, wherein the algorithm and the predetermined cutoff value has
i) a sensitivity for identifying kidney transplant rejection of at least about 93%;
ii) a specificity for identifying kidney transplant rejection of at least about 48%; and
iii) a negative predictive value for identifying kidney transplant rejection of at least about 97%.

23. The method of any one of claims 15-21, wherein the algorithm and the predetermined cutoff value has
i) a sensitivity for identifying kidney transplant rejection of at least about 61%;
ii) a specificity for identifying kidney transplant rejection of at least about 84%; and
iii) a negative predictive value for identifying kidney transplant rejection of at least about 90%.

24. A method of determining the risk of antibody-mediated rejection (ABMR) in a subject that has undergone a kidney transplant, the method comprising:
a) determining the expression level of at least two of five biomarkers and at least one endogenous control gene in RNA isolated from a biological sample from the subject,
wherein the five biomarkers comprise IL18BP, CXCL11, CD74, CD44 and C3;
b) normalizing the expression of the biomarkers to the expression level of the at least one endogenous control gene;
c) inputting the expression levels from step (b) into an algorithm to generate a score;
d) identifying the risk of ABMR in the subject based on the score.

25. The method of claim 24, wherein step (d) comprises:
i) comparing the score to a predetermined cutoff value; and
ii) identifying the risk of ABMR in the subject based on relationship between
the score and the predetermined cutoff value.

26. The method of claim 24 or claim 25, wherein the subject is a subject who has been identified as having a kidney transplant rejection or who has been identified as being at high risk for kidney transplant rejection.

27. The method of any one of claims 24-26, wherein step (a) comprises determining the expression level:
a) at least three of the five biomarkers; or
b) at least four of the five biomarkers.

28. The method of any one of claims 24-27, wherein step (a) comprises determining the expression level of each of the five biomarkers.

29. The method of any one of claims 24-28, wherein the algorithm is a product of a feature selection wrapper algorithm, machine learning algorithm, trained classifier, logistic regression model or any combination thereof, that was trained using: a) the expression levels of the at least one, or the at least two, or the at least three, or the at least four or each of the biomarkers in at least one biological sample from at least one subject who has ABMR; and b) the expression levels of the at least one, or the at least two, or the at least three, or the at least four or each of the biomarkers in at least one biological sample from at least one subject who has TCMR.

30. The method of any one of claims 24-29, wherein the algorithm and the predetermined cutoff value has
i) a sensitivity for ruling out ABMR of at least about 77%;
ii) a specificity for ruling out ABMR of at least about 62%;
iii) a negative predictive value for ruling out ABMR of at least about 90%;
iv) a positive predictive value for ruling out ABMR of at least about 38%.

31. The method of any one of the preceding claims, wherein the predictive classification algorithm, the feature selection wrapper algorithm, and/or the machine learning algorithm comprises XGBoost (XGB), random forest (RF), Lasso and Elastic-Net Regularized Generalized Linear Models (glmnet), Linear Discriminant Analysis (LDA), cforest, classification and regression tree (CART), treebag, k nearest-neighbor (knn), neural network (nnet), support vector machine-radial (SVM-radial), support vector machine-linear (SVM-linear), naïve Bayes (NB), multilayer perceptron (mlp), Boruta or any combination thereof.

32. The method of any one of the preceding claims, wherein the RNA isolated from a biological sample from the subject and/or the RNA isolated from a training set of biological samples comprises cell-free RNA, microvesicular RNA or any combination thereof.

33. The method of any one of the preceding claims, wherein the biological sample from the subject and/or the biological samples in the training sets is/are urine samples.

34. The method of any one of the preceding claims, wherein the at least one endogenous control gene comprises PGK1.

35. The method of any one of the preceding claims, wherein determining the expression level of a biomarker comprises quantitative PCR (qPCR), quantitative real-time PCR, semi-quantitative real-time PCR, reverse transcription PCR (RT-PCR), reverse transcription quantitative PCR (qRT-PCR), microarray analysis, sequencing, next-generation sequencing (NGS), high-throughput sequencing, direct-analysis, droplet digital PCR, or any combination thereof.

36. The method of any one of the preceding claims, further comprising:
i) performing a kidney biopsy on the subject;
ii) administering at least one kidney transplant rejection therapy to the subject;
iii) administering at least one TCMR-targeted therapy to the subject;
iv) administering at least one ABMR-targeted therapy to the subject; and/or
v) administering at least one kidney inflammation therapy to the subject.
